(19) United States
(12) Patent Application Publication
(10) Pub. No.: US 2014/0227708 A1
(43) Pub. Date: Aug. 14, 2014

(54) METHODS AND KITS USED IN IDENTIFYING MICRORNA TARGETS

(71) Applicant: Xiaolu Lim Ang Cambronne, West Linn, OR (US)

(72) Inventor: Xiaolu Lim Ang Cambronne, West Linn, OR (US)

(73) Assignee: OREGON HEALTH & SCIENCE UNIVERSITY, Portland, OR (US)

(21) Appl. No.: 14/236,619

(22) PCT Filed: Sep. 14, 2012
(86) PCT No.: PCT/US12/55353

§ 371 Date: Jan. 31, 2014

Related U.S. Application Data
(60) Provisional application No. 61/535,824, filed on Sep. 16, 2011.

(51) Int. Cl. C12Q 1/68

(52) U.S. Cl. CPC C12Q 1/686; C12Q 2531/113
USPC 435/6.12

(57) ABSTRACT

Described herein are methods and kits used to identify an endogenously expressed target mRNA of a microRNA of interest. The method involves the use of a dominant negative GW182 polypeptide that forms a stable complex with the target mRNA. The method further involves purifying the complex and identifying the target mRNA.

### PRIORITY CLAIM

This application claims priority to U.S. Provisional Patent Application No. 61/535,824, filed on 16 Sep. 2011, which is incorporated by reference in its entirety.

### ACKNOWLEDGEMENT OF GOVERNMENT SUPPORT

This invention was made with government support under Grant number RC1-NS067811 awarded by the National Institutes of Health. The government has certain rights in the invention.

### FIELD

Generally, this disclosure relates to methods of identifying the mRNA targets of a particular microRNA and more specifically to methods of identifying mRNA targets of microRNA using a dominant negative GW182 polypeptide.

### BACKGROUND

A microRNA (interchangeably referred to both in the art and herein as a miRNA or miR) is an RNA molecule, often between 20 and 30 nucleotides in length. MicroRNAs are endogenously expressed by eukaryotes and have been recognized to be key factors in the regulation of gene expression. In general, a microRNA mediates the silencing of a mRNA target through the recruitment of components of an RNA-protein assembly known as a RISC (an acronym for RNA-induced silencing complex). Depending on the exact components of the RISC, silencing may be mediated either through translational repression, decay or degradation of the mRNA transcript, or through direct cleavage of the mRNA transcript. (Czech B and Hannon G, Nat Rev Genet 12, 19-31 (2011), incorporated by reference herein.)

MicroRNAs are originally expressed as a primary microRNA transcript, and processed to their mature form as a single stranded small RNA molecule. Prior to target recognition, an active microRNA guide strand is incorporated into a functional RISC, while the complement of the active microRNA (the passenger or star strand) is discarded. (Kawamata T and Tomari Y, Trends in Biochemical Sciences 35, 368-376 (2010), incorporated by reference herein).

### SUMMARY

MicroRNAs are involved in the regulation of the biological pathways that characterize both healthy and diseased states. Each microRNA is capable of regulating potentially hundreds of mRNAs. Bioinformatic and computational prediction approaches to predict mRNA targets of microRNAs suffer from a relatively high number of false-positive and false-negative predictions because known recognition sites tend to be short and occur by chance. One of the problems with existing methods to empirically identify direct microRNA targets of microRNA is that many bona fide targets are actively downregulated through miRNA destabilization. Recent analyses estimate that as much as 84% of the effect of microRNAs on protein expression is mediated by microRNA destabilization (Guo et al, 2010 infra). As a result, current methods such as Ago-2 immunoprecipitation (Ago2-IP or RIP-SEQ) or CLIP-Seq will not detect or will underrepresent many important miRNA-mRNA interactions. The method disclosed herein involves stabilization of actively downregulated targets to provide robust target identification, a high signal to noise ratio, and no need to account for varying input levels of transcripts of enrichment of canonical seed sites. Furthermore, a single experiment using the method disclosed herein can capture miRNA targets mediated by multiple Argonaute family members and is not limited specifically to associations with Ago2. The disclosed method is therefore applicable to a wide range of microRNAs and target mRNAs.

One embodiment involves contacting a microRNA of interest with a polypeptide that is a member of the GW182 family of proteins. The mutant GW182 polypeptide comprises a mutation that renders it dominant-negative. It is also referred to herein as a dominant negative GW182 polypeptide or dnGW182. This contacting may be performed within a cell and the target mRNA may be an endogenously expressed mRNA. This embodiment further involves purifying a RISC complex comprising the dominant negative GW182 polypeptide and the target mRNA and identifying the target mRNA. The mutation may be any mutation in the GW182 polypeptide that causes it to be a dominant-negative GW182 polypeptide such as a mutation in the silencing domain, including an amino acid substitution mutation or a deletion mutation of one or more amino acids, up to and including the deletion of the entire silencing domain.

Another embodiment provides a kit that facilitates the methods described herein. The kit comprises a nucleic acid construct with a sequence that encodes a dominant negative GW182 polypeptide. The kit may further comprise a reagent that can be used to purify a RISC complex comprising the dominant negative GW182 polypeptide and a target mRNA. The kit may further comprise another reagent that can be used in the identification of the target mRNA. In some aspects, the reagent used to purify the complex binds specifically to the polypeptide or to a label to which the polypeptide is bound. In some aspects, the reagent used to identify the target mRNA is an oligonucleotide that may be used to identify the mRNA of interest using polymerase chain reaction, nucleic acid sequencing (including next generation sequencing) and/or hybridization to a microarray. In other aspects, the kit may comprise instructions that describe the performance of the method. In still other aspects, the kit may comprise a microRNA of interest.

The foregoing and other features will become more apparent from the following detailed description which proceeds with reference to the accompanying figures.

### BRIEF DESCRIPTION OF THE DRAWINGS

FIG. 1 depicts a schematic of the cassette encoding synthetic targets to monitor miR-132-RISC.

FIG. 2 depicts the relative abundance of GFP and dsRed transcripts in the presence of miR-132 and dnGW182 as indicated, assessed by qPCR (2^{(ΔCt)}) and normalized to Gapdh as described below. All cells were transfected with the construct shown in FIG. 1 and described in Example 2. “miR-Scrm,” indicates that the miRNA of interest was a negative control (scrambled) microRNA; “vector” indicates that no dominant negative GW182 polypeptide was expressed in the system; miR-132 indicates that microRNA number 132 was present; therefore expression of GFP in the experimental system was silenced. TNRC6A^{DN} indicates that a dominant negative form of human TNRC6A (SEQ ID NO: 2) was expressed in the cells.

FIG. 3 is an image of a Western blot indicating that TNRC6A^{DN} is capable of forming a stable complex comprising Argonaute 2 (Ago2). The label at the top of the blot indicates the expressed construct (either FLAG-HA or FLAG-HA-TNRC6A^{DN}). The label to the far left indicates the immunoprecipitation conditions (either a FLAG-agarose immunoprecipitation or the input diluted to 4% (no immunoprecipitation)). The label on the inner left indicates the antibody used in detection of the Western blot (anti-FLAG-M2, anti-Ago2).

FIG. 4 is an image of a Western blot indicating that TNRC6A^{DN} is capable of forming a complex with both Ago1 and Ago2. The topmost, underlined labels indicate the immunoprecipitation conditions (either the input diluted to 10% (no immunoprecipitation) or a FLAG-agarose immunoprecipitation.) The top labels nearest to the blot indicate with a ‘+’ the constructs expressed by the HEK293T cells (FLAG-vector alone, FLAG-TNRC6A^{DN} (indicated here as GW182^{DN}), or FLAG-PTBP2). PTBP2 is polypyrimidine tract binding protein 2. Labels to the right of the blot indicate the antibody used in detection of the Western blot (anti-Ago1, anti-Ago2, anti-GAPDH, and anti-FLAG). GAPDH is glyceraldehyde 3-phosphate dehydrogenase. Labels to the left of the blot with arrows indicate the relative positions of FLAG-TNRC6A^{DN} (indicated here as GW182^{DN}) and FLAG-PTBP2.

FIG. 5 is an image of a Western blot indicating that TNRC6A^{DN} is capable of forming a stable RISC complex with endogenous target mRNA. The topmost labels at a 45° angle indicate which lanes contain lysates from 293T cells that express FLAG-HA-TNRC6A^{DN}. The others do not. The next labels on top, closest to the blot (vertically oriented) indicate the microRNA expressed by the cells (either a scrambled, negative control microRNA (Scrm) or microRNA-132). The labels on the right of the blot indicate the detection antibody (anti-FLAG (here as FLAG-HA-TNRC6^{DN}) anti-Ago2, and anti-GAPDH. The labels on the bottom of the blot indicate the immunoprecipitation conditions—either the non-immunoprecipitated inputs or anti-FLAG immunoprecipitation.

FIG. 6 is an image of a Western blot indicating that a complex comprising TNRC6A^{DN} may also be immunoprecipitated with Ago2. The topmost, underlined labels indicate the inputs to the Western blot (either an anti-myc immunoprecipitation diluted to 20% or the input diluted to 2% (no immunoprecipitation.)) The next label down from the top indicates with a T-FT which lanes are lysates from HEK293T cells that were transfected with pCMV-FLAG-HA-TNRC6A^{DN}. The next set of labels from the top (vertically oriented) indicates the microRNA expressed by the cells (either a scrambled, negative control microRNA (Scrm oligo) or microRNA-132 (miR-132). The labels on the left of the blot indicate the detection antibody used in the Western blot (anti-FLAG-M2, anti-myc, or anti-GAPDH). The labels on the right of the blot indicate with arrows the position of FLAG-HA-TNRC6A^{DN}.

FIG. 7 is an image of a Western blot indicating that endogenous p21 expression in HEK293T cells is affected by microRNA-132. The top label indicates with a ‘+’ which cells were transfected with a microRNA-132 mimic (miR-132 mimic). The labels to the left indicate the detection antibody (either anti-p21 or anti-alpha tubulin). The labels on the bottom indicate well numbering.

FIG. 8 is an image of a Western blot indicating that TNRC6A^{DN} forms a stable complex with miR-132 and endogenously expressed p21 target mRNA that does not mediate miRNA silencing. The topmost, underlined labels indicate whether or not the HEK293T cells were transfected with pcDNA (negative control) or TNRC6A^{DN}. The next labels to the top of the blot (vertically oriented) indicate the microRNA expressed by the cells (either a scrambled, negative control microRNA (miR-scrm) or microRNA-132 (miR-132). The labels to the left of the blot indicate the detection antibody used in the Western Blot (either anti-p21 or anti-GAPDH). The numbers at the bottom of the blot indicate well numbering.

FIG. 9 is a diagram that indicates the strategy used to generate the data of FIG. 10. HEK293 cells stably transfected with the red/green construct described in Example 2 are transfected with microRNA-124, or microRNA-132. Because the Red/Green construct causes silencing of GFP expression in the presence of miR-132, GFP expression may be used as a microRNA transfection control. MicroRNA-124 has no effect on GFP or DsRedEx1 expression. The cell lines are also transfected with FLAG-tagged TNRC6A^{DN} and immunoprecipitated with an anti-FLAG reagent (with nonimmunoprecipitated inputs included as a negative control). Total RNA is eluted from each sample and the target mRNA's identified by quantitative reverse transcription PCR.

FIG. 10 is a bar graph that indicates that TNRC6A^{DN} is capable of forming a complex with a microRNA of interest and an endogenous target mRNA and that the complex may be purified and that the target mRNA of the microRNA of interest may be identified. Bars of the indicated patterns are identified in the inset of the graph. The top (light gray) bar indicates the results from cells expressing microRNA-132 with no immunoprecipitation. The second (darker gray) bar indicates the results from cells expressing microRNA-124 with no immunoprecipitation. The third (white) bar indicates the results from cells expressing microRNA-132 with FLAG-immunoprecipitation of the complex. The fourth (black) bar indicates the results from cells expressing microRNA-124 with FLAG-immunoprecipitation of the complex. Labels at the bottom are the indicated target mRNAs.

FIG. 11 is a diagram that indicates the strategy used to detect mRNA targets of microRNA-132, microRNA-181 and microRNA-124 by high throughput or next generation sequencing.

FIG. 12 is an image of a Western blot from an experiment described by the diagram of FIG. 11 and described in Example 7. The topmost, underlined labels indicate the inputs (either a FLAG immunoprecipitation diluted to 5% or the inputs to the immunoprecipitation diluted to 0.25%). The second set of labels on top of the blot and to the left indicate (with a ‘+’ sign) the microRNA of interest (a scrambled, negative control microRNA (Scrm oligo), microRNA-132, or microRNA-124.) The labels directly to the left of the blot indicate the detection antibody used in the Western blot (anti-Flag, anti-Ago2, or Anti-GAPDH). The label to the right of the blot indicates the position of TNRC6A^{DN} (indicated as FLAG-HA^{DN}GW182.) Numbers at the bottom of the gel indicate sequential wells.

FIG. 13 is a bar graph indicating the relative enrichment of each of the indicated mRNA by each indicated miRNA of interest relative to negative controls. Negative control microRNA (miR-Scrm) is indicated by black bars. MicroRNA-132 is indicated by white bars, and microRNA-124 is indicated by spotted bars. Data from FIG. 10 for expression of GFP target mRNA and endogenous Ctdsp1 mRNA is recapitulated in these samples prepared for DNA sequencing.

FIG. 14 is an image of a simulated gel that is the output of a capillary electrophoresis instrument, indicating proper preparation of mRNA libraries for next generation sequencing.

FIG. 15 is an image of a Flag-dnGW182 associated with endogenous Argonaute family members with both miR-124 and miR-132 as miRNA of interest.

FIG. 16 is a bar graph showing the results using bidirectional 2-color sensors (FIG. 1) for miR-132 and control cel-miR-239b to monitor endogenous miR-132 activity in HEK293T cells (gray). The response for endogenous miR-132 was confirmed by transfection of 50 μM of 2′OMe-AS-miR-132 antisense inhibitor. 2′OMe-AS-miR-1 was used as a negative control for inhibition of miR-132.

FIG. 17 is a plot showing the results of ratiometic analysis using flow cytometry on the bidirectional 2-color sensors of FIG. 1. The results revealed that dnGW182 stabilizes GFP target transcripts without silencing, thereby resulting in increased GFP expression in cells transfected with both miR-132 and dnGW182 (red.)

FIG. 18 is a bar graph showing the number of total and uniquely mapped reads for each of the indicated miRNAs of interest from next generation sequencing of target mRNA resulting from each screen. Each of the three labeled bars indicates a separate RISCtrap screen using the particular miRNA of interest. On average, 40-50 million 100 bp single reads were obtained per RISCtrap sample and approximately 75% were uniquely mapped using Top Hat—with a human GRCh37/hg19 reference genome and RefSeq gene annotation guidance program.

FIG. 19 is a set of six plots (labeled FIGS. 19A through 19F) outlining the analysis of the reads resulting from the sequencing of the target mRNA obtained through RISCtrap screening. Data dimensionality was reduced using principal components analysis (PCA) to transform a set of correlated variables into a smaller set of uncorrelated variables. PCA is useful for identifying patterns in data and clustering datasets with similar conditions. FIGS. 19A and 19D indicate the raw data. The first component (PC1) describes the largest contribution to variability in the original data, while the second component (PC2) describes the next largest contribution to variability, etc. Read counts were assigned to each gene according to the RefSeq annotation. Non-polyadenylated genes and targets with read counts less than 200 across all samples were bioinformatically filtered (about 11,800 targets) (FIGS. 19B and 19E). The median of the geometric mean of the remaining 10,885 genes was then used for normalization (FIGS. 19C and 19F). At each step, datasets were analyzed by PCA to ensure that the clustering characteristics were maintained (FIG. 19A, FIG. 19B and FIG. 19C). Meanwhile, violin plots were employed to visualize the data shape and distribution (FIG. 19D, FIG. 19E and FIG. 19F).

FIG. 20 is a set of six plots showing the estimation of variance and differential enrichment for pairwise comparisons. The relationship between the data variance (dispersion) and mean is estimated in DESeq. Empirical dispersion values (black dots) were plotted against the normalized mean values per gene, with the fitted dispersion plotted as a red line (top). Differential enriched genes in pairwise comparisons were determined by ANOVA within the DESeq package, and significantly enriched genes were plotted as red, green or blue dots in the corresponding graphs (bottom).

FIG. 21 is a bar graph depicting an assessment of miR-124 targets not identified by RISCtrap, but identified by another method of assessing target mRNA of microRNAs of interest for binding to RISC. Thirteen transcripts were selected for examination by qPCR for co-purification with RISC containing a scrambled miRNA, miR-132, and mir-124 in the presence of Flag-dnGW182. Input RNA (100 ng) was also analyzed to confirm that the transcripts were expressed in HEK293T cells. Quantitative PCR primers for Ctdsp1 and Gapdh were included as positive and negative controls, respectively. Values were normalized to Gapdh.

FIG. 22 is an image of a heat map showing all target mRNAs identified from the RISCtrap screens using miR-124, miR-132, and miR-181 organized by biological replicates. (ANOVA, FDR<0.15. Log₂ fold-enrichment≧1.) Selected known target mRNAs are indicated and examples of novel miR-132 targets are highlighted in yellow.

FIG. 23 is a Venn Diagram showing the overlap of target mRNAs between those sets of target mRNAs identified by screens of miR-181, miR-124, and miR-132.

FIG. 24 is a three-dimensional plot showing that microRNA target datasets showed distinct fold-enrichments (log₂). MiR-181d targets (green); miR-124 targets (red), miR-132 targets (blue). Targets predicted to be co-regulated are depicted by shared colors.

FIG. 25 is a bar graph showing the validation of selected target mRNAs that were highly enriched in the RISCtrap screen by quantitative PCR.

FIG. 26 is a bar graph showing the validation of selected target mRNAs that were moderately enriched in the RISCtrap screen by quantitative PCR.

FIG. 27 is a bar graph showing the validation of target mRNAs that were modestly enriched in the RISCtrap screen by quantitative PCR. mRNA transcripts that were identified as not being enriched are outlined in yellow on the far right of the graph and were also validated by quantitative PCR.

For all of FIGS. 25, 26, and 27, targets of miR-181 are shown in green, targets of miR-124 are shown in red, and targets of miR-132 are shown in blue.

FIG. 28 is a bar graph showing the percentages of transcripts in each dataset classified by the inclusion of at least 1 MRE motif and the distribution of motif types. Each transcript is counted only once and classified according to inclusion of the following motifs in this order: 8-mer>7mer-m8>7mer-a1>6-mer>pivot.

FIG. 29 is a set of three plots showing the cumulative MRE frequencies for each target dataset, based on inclusion of specific MREs as defined in FIG. 28. Data is plotted by the observed fold-enrichment (Log₂) observed in the RISCtrap screen. Nontarget transcripts that did not contain any seed sites (black) did not enrich (median≈1). Targets for miR-132, miR-124, and miR-181 contained a variety of MRE sites and all tended to enriched between 2.5-3.0 fold, except for the mir-181 targets with reiterated 8-mers that averaged 5.5 fold enrichment. Targets that contained only a pivot MRE were excluded from this analysis because the number of targets was less than 3 in each case.

FIG. 30 is a bar graph depicting the total number of 7mer-m8 (light grey) and pivot (dark grey) MRE motifs for each miRNA dataset compared to the number of targets (black bars).

FIG. 31 is a bar graph depicting the mean number of 7mer-m8 MRE motifs per target for each microRNA dataset.

FIG. 32 is a bar graph depicting the distribution of candidate MREs for each microRNA target dataset, based on its position in the target's 5′UTR, open reading frame (ORF), or 3′UTR.

FIG. 33 is a set of two bar graphs depicting the frequency of MRE motifs plotted against the relative position of MRE in the 3′-UTR (miR-132 left, miR-124 right).

FIG. 34 is a set of three sequence plots depicting the results of de novo MEME analysis using all targets from the miR-124 and miR-181 target mRNA datasets. The analysis revealed canonical MRE motifs in the 3′-UTR of miR-124 targets (296 motifs, p=2.6×10⁻¹⁰⁸), in the 3′UTR of miR-181 targets (151 motifs, p=1.3×10⁻⁵⁴), and in the ORF of miR-181 targets (1000 motifs, p=9.4×10⁻¹⁴⁸⁸).

FIG. 35 is a sequence alignment showing well-conserved miR-132 MRE sequences are found in the 3′UTR of both novel candidate targets CRK and TJAP1.

FIG. 36 is a bar graph depicting the results of luciferase assays in HEK293T cells demonstrated that each of their 3′-UTR sequences conferred regulation by miR-132. Mutation of the predicted MRE (mutated sequences are bold and underlined in FIG. 35) blocked miR-132 regulation.

FIG. 37 is an image of Western blots of whole cell lysates from forebrains of litter matched siblings of miR-132(+/+) and miR-132 (−/−) mice. Lysates were probed for endogenous protein levels of novel targets CRK and TJAP1, as well as known target Hb-EGF, and negative controls DHHC9, αTubulin, and GAPDH.

### SEQUENCE LISTING

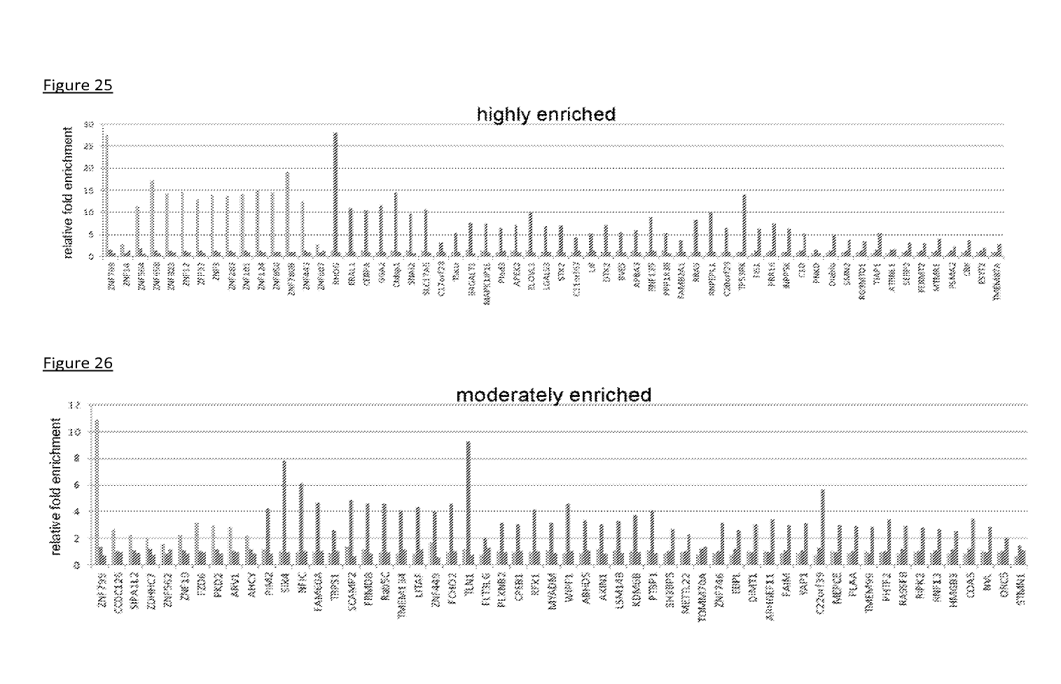

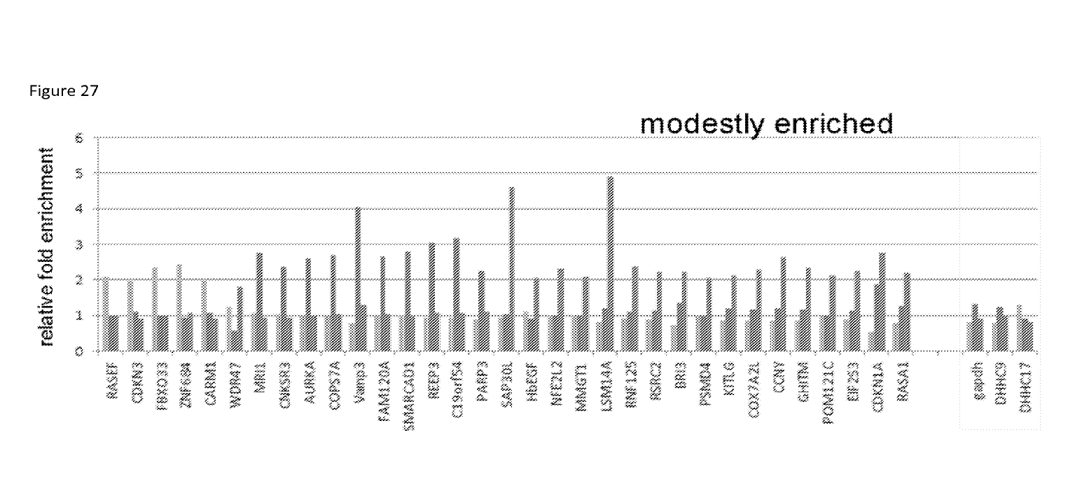

SEQ ID NO: 01 is an amino acid sequence of a full-length D. melanogaster GW182.
SEQ ID NO: 02 is an amino acid sequence of a full-length human TNRC6A.
SEQ ID NO: 03 is an amino acid sequence of a full-length human TNRC6B isoform 1.
SEQ ID NO: 04 is an amino acid sequence of a full-length human TNRC6B isoform 2.
SEQ ID NO: 05 is an amino acid sequence of a full-length human TNRC6B isoform 3.
SEQ ID NO: 06 is an amino acid sequence of a full-length human TNRC6C isoform 1.
SEQ ID NO: 07 is an amino acid sequence of a full-length human TNRC6C isoform 2.
SEQ ID NO: 08 is an amino acid sequence of a dominant-negative D. melanogaster GW182.
SEQ ID NO: 09 is an amino acid sequence of a dominant-negative human TNRC6A.
SEQ ID NO: 10 is an amino acid sequence of a dominant negative human TNRC6B.
SEQ ID NO: 11 is an amino acid sequence of a dominant negative human TNRC6C.
SEQ ID NO: 12 is a nucleic acid sequence of a dominant-negative D. melanogaster GW182.
SEQ ID NO: 13 is a nucleic acid sequence of a dominant-negative human TNRC6A.
SEQ ID NO: 14 is a nucleic acid sequence of a dominant-negative human TNRC6B.
SEQ ID NO: 15 is a nucleic acid sequence of a dominant-negative human TNRC6C.
SEQ ID NO: 16 is a nucleic acid sequence of a construct configured to express dominant-negative human TNRC6A using an adenoviral expression system.
SEQ ID NO: 17 is a nucleic acid sequence of a construct configured to express dominant-negative human TNRC6B using an adenoviral expression system.
SEQ ID NO: 18 is a nucleic acid sequence of a construct configured to express dominant-negative human TNRC6C using an adenoviral expression system.
SEQ ID NO: 19 is a nucleic acid sequence of a construct configured to express dominant-negative human TNRC6A that may be labeled with biotin.
SEQ ID NO: 20 is a nucleic acid sequence of a construct configured to express dominant-negative human TNRC6B that may be labeled with biotin.
SEQ ID NO: 21 is a nucleic acid sequence of a construct configured to express dominant-negative human TNRC6C that may be labeled with biotin.
SEQ ID NO: 22 is a nucleic acid sequence of a construct configured to cause a cell to express dominant-negative human TNRC6A labeled with both a FLAG-tag and a His-Tag.
SEQ ID NO: 23 is a nucleic acid sequence of a construct configured to express dominant-negative human TNRC6A labeled with a myc tag.
SEQ ID NO: 24 is a nucleic acid sequence of a construct configured to express dominant-negative human TNRC6B labeled with a myc tag.
SEQ ID NO: 25 is a nucleic acid sequence of a construct configured to express dominant-negative human TNRC6C labeled with a myc tag.
SEQ ID NO: 26 is a nucleic acid sequence of a construct configured to express dominant negative human TNRC6A in a lentiviral pHAGE-N-Flag-HA vector.
SEQ ID NO: 27 is a nucleic acid sequence of a construct configured to express dominant negative human TNRC6B in a lentiviral pHAGE-N-Flag-HA vector.
SEQ ID NO: 28 is a nucleic acid sequence of a construct configured to express dominant negative human TNRC6C in a lentiviral pHAGE-N-Flag-HA vector.
SEQ ID NO: 29 is an amino acid sequence of human Argonaute-1 (Ago1).
SEQ ID NO: 30 is an amino acid sequence of human Argonaute-2 (Ago2).
SEQ ID NO: 31 is a nucleotide sequence of a mature human microRNA-132.
SEQ ID NO: 32 is a nucleotide sequence of a mature human microRNA-124.
SEQ ID NO: 33 is a nucleotide sequence of a mature human microRNA-181d
SEQ ID NO: 34 is a nucleotide sequence of a negative control microRNA sequence (miR-scrm).
SEQ ID NO: 35 is a nucleotide sequence of an oligo-dT-T7 primer.
SEQ ID NOs: 36-447 are nucleotide sequences of qPCR primers used to verify enrichment of RISCtrap—data shown in FIGS. 25, 26, and 27.

### DETAILED DESCRIPTION

Disclosed herein is a method of identifying an endogenously expressed messenger RNA (mRNA) target of a microRNA of interest and kits that facilitate the use of the method. Identifying mRNA targets of microRNA is one of the most demanding problems in the field of study of microRNA and there is a long felt need to accurately identify those targets.

Each miRNA can target potentially hundreds of mRNA transcripts, thus one of the most important challenges is to identify the cohort of target mRNAs regulated by a particular microRNA in a cell. Global analyses have demonstrated that individual miRNAs can have substantial impact on regulated targets at the transcriptome level (Baek D et al, Nature 455, 64-71 (2008); Eulalio A et al RNA 15, 21-32 (2009); Guo H et al, Nature 466, 835-840 (2010); Hendrickson D G et al, PLoS One 3, e2126 (2008); Lim L P et al, Nature 433, 769-773 (2005); and Selbach M et al, Nature 455, 58-63 (2008), all of which are incorporated by reference herein.) Multiple studies have culminated in the identification of a conserved mechanism for mRNA destabilization through the actions of GW182/hTNRC6 family members. These studies demonstrated that GW182 is recruited to targeted transcripts as a core component of RISC through a direct interaction between its N-terminal domain and Argonaute. (Eulalio A et al, RNA 15, 1067-1077 (2009); Eulalio A et al, Nat Struct Mol Biol 15, 346-353 (2008); Lazzaretti D et al, RNA 15, 1059-1066 (2009); Yao B et al, Nucleic Acids Res 39, 2534-2547 (2010); Zipprich J T et al, RNA 15, 781-793 (2009); Behm-Ansmant I et al, Genes Dev, 20, 1885-1898 (2006) all of which are incorporated by reference herein). GW182 then binds to polyadenylate-binding protein 1 (PABP). This GW182-PABP interaction disrupts cap-dependent translation and allows GW182 to directly recruit cytoplasmic deadenylase complexes CAF1/Not1/CCR4 and PAN2-PAN3, which then deadenylate the transcript resulting in its destabilization and decay (Behm-Ansmant I et al, Genes Dev, 20, 1885-1898 (2006); Braun J E et al, Mol Cell 44, 120-133 (2011); Chekylaeva M et al, Nat Struct Mol Biol, 18, 1218-1226 (2011); Fabian M R et al, Nat Struct Mol Biol, 18, 1211-1217; Fabian M R et al, Mol Cell, 35, 868-880 (2009); Huntzinger E et al, EMBO J, 29, 4146-4160 (2010). Jinek M et al, Nat Struct Mol Biol, 17, 238-240 (2010); Kuzuoglu-Ozturb D, et al, Nucleic Acids Res 12, 5651-5665 (2012); Zekri L et al, Mol Cell Biol, 29, 6220-6231 (2009) all of which are incorporated by reference herein). Additional in vitro and cell-based studies have provided evidence that translational repression is often coupled to and precedes mRNA destabilization (Djuranovic S et al, Science 336, 237-240 (2012); Fabian M R et al, Mol Cell 35, 868-880 (2009); Hendrickson D G et al, PLoS Biol 7, e1000238 (2009) and Moretti F et al, Nat Struct Mol Biol 19, 603-608 (2012); all of which are incorporated by reference herein.)

As a result, many endogenously expressed target mRNA transcripts of an miRNA of interest—which may be present at low abundance due to mRNA destabilization—could be missed or under-represented with current approaches to detect miRNA-mRNA interactions such as Ago2 immunoprecipitations, PAR-CLIP, or HITS-CLIP (Chi et al, Nature 460, 479-486 (2009); Hafner M et al, Cell 141, 129-141 (2010); Hendrickson et al 2008 supra; and Karginov et al, Proc Natl Acad Sci USA 104, 19291-19296 (2007); all of which are incorporated by reference herein.)

Bioinformatic target predictions of microRNA regulation are often unreliable because recognition is largely governed by cellular context, including the availability of a mature microRNA and accessibility of the MRE. Furthermore, target recognition involves noncontiguous base-pairing between the mature microRNA and a recognition sequence element (MRE) on the transcript, and requires the function of a large multimeric RNA-protein complex, namely the RISC. The exact and full spectrum of characteristics that govern microRNA target recognition are not fully understood. Thus, there is actually very little overlap among the results obtained by various algorithms used in current computational methods and therefore many microRNA-mRNA interactions predicted by computational models are not borne out by experimental data (Alexiou P et al, Bioinformatics 25, 3049-3055 (2009) hereby incorporated by reference.) Because computational methods have drawbacks, empirical methods are essential for identifying the target mRNA for any microRNA of interest.

There is a long-felt need for a robust screening method of mRNA targets of a microRNA of interest (Thomas M et al, Nature Struct Mol Bio 17, 1169-1174 (2010), hereby incorporated by reference). MicroRNA-dependent regulation of the transcriptome is generally characterized by translational silencing and decay of multiple mRNA targets. While analysis of changes in the proteome and transcriptome following alteration of a microRNA may reveal an effect of microRNA silencing on gene expression, it does not directly identify the target mRNA molecules of the microRNA of interest. That is, microarray analysis of microRNA silencing does not differentiate between those mRNAs that are silenced by the microRNA of interest and those that are downregulated due to downstream effects caused by that silencing. Moreover, it ignores other types of regulation that may independently cause changes in protein or RNA abundance, for example, transcriptional control or protein half-life.

Some have attempted to isolate mRNA targets of a microRNA of interest by immunoprecipitation of one or more components of the RISC complex. However, because microRNA silencing involves degradation of target mRNA, such techniques have been disappointing. Immunoprecipitation based on antibody affinity to the Argonaute (Ago) proteins is particularly difficult because mammals have as many as seven Ago proteins and it is possible that they may be used interchangeably by RISC. Other techniques incorporate mRNA-protein cross-linking such as in a method known as CLIP (Chi et al, Nature 460, 479-486 (2009); Hafner M et al, Cell 141, 129-141 (2010) both of which are incorporated by reference herein.) This approach still results in the identification of few actively targeted transcripts. CLIP also has the disadvantages of additional steps caused by performing the cross-linking, and that it is dependent on bioinformatics to assign target-transcript pairs based on known base pairing rules. Clearly, there is a need for a method that is capable of identifying the endogenous cellular mRNA targets of a microRNA of interest and for kits that facilitate the performance of such an assay method.

To overcome the challenges presented by identifying mRNA targets of miRNAs of interest, the RISCtrap method was developed. RISCtrap couples stabilization of target mRNA in a RISC complex with purification of RISC-miRNA-mRNA intermediates. Central to this strategy is the use of a dominant negative GW182 polypeptide (also referred to herein as dominant-negative GW182 or dnGW182). A dominant-negative GW182 cannot recruit effectors to silence and degrade the target mRNA. Transcripts are thus “trapped” in this intermediary protein-RNA complex and co-purified by immunoprecipitation of one or more components of the complex. The target mRNA is then identified by any of a number of methods including but not limited to amplification with gene-specific primers, cloning, microarray or DNA sequencing.

An miRNA of interest may be any miRNA that can silence one or more target mRNAs. The miRNA of interest can be expressed ectopically or introduced to a cell by transfection, such that the total cellular pool miRNA-RISC-target mRNA is skewed towards complexes comprising the miRNA of interest and endogenous target mRNA regulated by the miRNA of interest. Target mRNA that are enriched in the pool of mRNA isolated by purification of a RISC complex comprising the dominant negative GW182 relative to the pool of mRNA obtained with a different miRNA (such as a mutant, control, or unrelated miRNA) are identified as mRNA targets. The enrichment of a target mRNA may be more than 1.2 fold, more than 1.5 fold, more than 1.8 fold, or more than 2 fold relative to the amount of the same mRNA obtained in another pool to identify it as a target mRNA of the microRNA of interest.

It has been shown that GW182 polypeptides with deletions at the C-terminal silencing domain are dominant negative in that they inhibit protein translation and the release of an mRNA from a complex of microRNA, mRNA and dominant negative GW182 polypeptide. (Zekri L et al, Mol Cell Biol 29, 6220-6231 (2009), Balliat D and Shiekhattar R, Mol Cell Biol 29, 4144-4155, (2009) both of which are incorporated by reference herein). In particular a RNA-recognition motif (RRM) domain near the C-terminus has importance in regulating silencing (Balliat D and Shiekhattar R, 2009 supra). However, none of these mutant forms of GW182 polypeptides were shown to identify endogenously expressed target mRNAs.

In eukaryotes, the members of the GW182 family of proteins are components of RISC and are necessary for miRNA mediated silencing. In Drosophila, the GW182 polypeptide has an N-terminal region that interacts with the Argonaute family of proteins and has a silencing domain that is necessary for mediating silencing and for release of GW182 from RISC. (Zekri et al, supra). The mammalian forms of GW182 include TNRC6A, TNRC6B, and TNRC6C and these have been demonstrated to silence microRNA transcripts independently of the Ago proteins. In addition, in mammals, all TNRC6 variants can interact with as many as four Ago proteins. (Lazaretti et al, RNA 15, 1059-1066 (2009) incorporated by reference herein.)

A microRNA silences translation of one or more specific mRNA molecules by binding to a microRNA recognition element (MRE,) which is defined as any sequence that directly base pairs with and interacts with the microRNA somewhere on the mRNA transcript. Often, the MRE is present in the 3′ untranslated region (UTR) of the mRNA, but it may also be present in the coding sequence or in the 5′ UTR. MREs are not necessarily perfect complements to microRNAs, usually having only a few bases of complimentarity to the microRNA and often containing one or more mismatches within those bases of complimentarity. As a result, microRNA-mRNA interactions are difficult to predict. The MRE may be any sequence capable of being bound by a microRNA sufficiently that the translation of the target mRNA is repressed by a microRNA silencing mechanism such as the RISC.

A microRNA of interest is any microRNA molecule for which the identification of one or more target mRNAs is sought through the use of the disclosed methods. For example, a microRNA of interest may be transfected into a cell that expresses a dominant negative GW182. A microRNA of interest may target any number of target mRNAs, including 0, 1, 2 or more, 10 or more, 100 or more, or 500 or more target mRNAs. The identification of multiple target mRNAs, the quantification of one or more target mRNAs, and the identification of different target mRNA resulting from a change in conditions such as cell type, pretreatment with a drug compound or mutating one or more nucleotides of the microRNA of interest could be used to generate a profile of mRNA regulation by the microRNA of interest. Note that any synthetic, mutant, pathogenic, naturally occurring or non-naturally occurring microRNA could also be a microRNA of interest.

A target mRNA may be any ribonucleic acid molecule that results from the transcription of DNA template. It may comprise one or more introns, or one or more introns may have been spliced out of the mRNA and the splice sites rejoined. An unprocessed or partially processed mRNA may be termed pre-mRNA. A completely processed mRNA ready to be used in protein translation may be called a mature mRNA. An mRNA may be post-transcriptionally capped and/or polyadenylated in order to prime the transcript for active translation. A polyadenylated transcript refers to the addition of one or more adenine nucleotides to the 3′ end of the molecule after the transcription of DNA into RNA by an RNA polymerase. A target mRNA is any mRNA molecule that can be regulated by a microRNA of interest or any mRNA that is enriched in an mRNA pool resulting from purification of a protein complex comprising a dominant negative GW182 polypeptide and the microRNA of interest, relative to a control mRNA pool resulting from purification of a protein complex comprising a dominant negative GW182 and a control microRNA.

A target mRNA will often comprise at least one MRE (microRNA recognition element). Just as a single microRNA may regulate a number of different target mRNAs, a single target mRNA may be regulated by a number of different microRNAs. The concept of a target mRNA of a microRNA of interest also encompasses an mRNA that is subject to translational silencing by the microRNA, an mRNA that binds to the microRNA in one or more silencing complexes such as the RISC, or a microRNA that is identified as associating with the microRNA of interest using one or more of the disclosed methods. Preferably, the target mRNA is endogenously expressed by a cell.

An endogenously expressed mRNA is any mRNA expressed by a cell in a normal or perturbed state, but excludes any mRNA introduced into the cell by any human engineered DNA vector produced through the use of recombinant DNA techniques. In other words, an endogenously expressed mRNA is any mRNA that was not introduced into the cell by transfection, viral transduction using a recombinant, or otherwise human engineered virus, or any other experimental process. An endogenously expressed mRNA may be expressed by a cell undergoing any process of expansion (such as mitosis) or differentiation. An endogenously expressed mRNA may be any mRNA expressed by a resting cell. An endogenously expressed mRNA further encompasses mRNA expressed by a cell in response to a stimulus such as an environmental stressor (such as osmotic shock, oxygen deprivation, glucose deprivation, etc.), mRNA expressed by a cell in response to a stable and heritable modification of cell type and background, or mRNA expressed in response to an exogenously added composition (such as a pharmaceutical composition, receptor ligand, receptor antagonist, or receptor agonist.) An endogenously expressed mRNA may be an mRNA expressed by a cancer cell. An endogenously expressed mRNA may be expressed in response to a viral infection and may include mRNA from viral genes, so long as those genes were not introduced into the virus through recombinant DNA technology. An endogenously expressed mRNA may be an mRNA that is known to be regulated by the microRNA of interest, it may be an mRNA that is not known to be regulated by the microRNA of interest, or it could be an mRNA that is still undiscovered (in that it had never been detected before.)

The method may comprise contacting the microRNA of interest with a dominant negative GW182 polypeptide. A dominant negative GW182 polypeptide is a polypeptide that is related to other members of the GW182 family by sequence homology that has the following characteristics: (1) it is capable of forming a complex comprising itself, the target mRNA, and the microRNA of interest, and (2) it renders the complex incapable of performing microRNA silencing. Members of the GW182 family are highly conserved among animals and share functional activity in that many members of the family have homologous sequences that bind Ago protein, homologous sequences that mediate silencing, homologous regions that interact with other members of RISC, etc. So the polypeptide may be identified based upon its sequence homology with one or more members of the GW182 family of proteins or a dominant negative form thereof. For example, the dominant negative GW182 polypeptide may share at least 30%, at least 40%, at least 50%, at least 60%, at least 70%, at least 80%, at least 90%, at least 95%, at least 99%, or at least 99.99% sequence homology with Drosophila GW182 (in this case isoform A) (SEQ ID NO: 01). In another example, the dominant negative GW182 polypeptide may share at least 30%, at least 40%, at least 50%, at least 60%, at least 70%, at least 80%, at least 90%, at least 95%, at least 99% or at least 99.99% sequence homology with any dominant negative GW182 polypeptide such as a dominant negative human TNRC6A, TNRC6B, and TNRC6C or any isoform thereof (for example, SEQ ID NOs: 02-07).

Sequence homology between two or more nucleic acid sequences or two or more amino acid sequences, may be expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence. 1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75). For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). In addition, a manual alignment can be performed. Proteins with even greater similarity will show increasing percentage identities when assessed by this method, such as at least about 50%, 60%, 70%, 75%, 80%, 85%, 90%, 95%, 98%, 99%, or 100% sequence identity.

When aligning short peptides (fewer than around 30 amino acids), the alignment is to be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 50%, 60%, 70%, 75%, 80%, 85%, 90%, 95%, 98%, or 99% sequence identity to a protein. When less than the entire sequence is being compared for sequence identity, including a comparison of a dominant negative GW182 polypeptide, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

A dominant negative form of a protein is one that is mutated relative to the wild type of the protein in such a way that it acts in opposition to the operation of the wild type protein within the cell. For example, if a protein is only active as a dimer or a trimer, a mutant form of a protein that would be capable of combining with the active form of the protein, but lacks signaling capacity would be a dominant negative form of the protein because functional multimers would not form. With regard to dominant negative GW182 polypeptides, a dominant negative form of the protein is one that forms a complex comprising the protein itself, a microRNA and a target mRNA, but wherein the resultant complex is incapable of performing microRNA silencing. In some examples, the dominant negative GW182 polypeptide comprises a mutation in its silencing domain.

A mutation may refer to any difference in the sequence of a biomolecule relative to a reference or consensus sequence of that biomolecule. A mutation may be observed in a nucleic acid sequence or a protein sequence. Such a reference or consensus sequence may be referred to as “wild type”. A mutation in a nucleic acid relative to a wild type may result in a reduction in function of the expressed protein or nucleic acid, a gain in function of the expressed protein or nucleic acid, no change in function of the protein or nucleic acid, a disease, a selective advantage, a selective disadvantage, or any other molecular, cellular, or organismal, effect.

A mutation may comprise any of a number of changes alone or in combination. Some types of mutations include point mutations (differences in individual nucleotides or amino acids); silent mutations (differences in nucleotides that do not result in an amino acid changes); deletions (differences in which one or more nucleotides or amino acids are missing); frameshift mutations (differences in which deletion of a number of nucleotides indivisible by 3 results in an alteration of the amino acid sequence); and any other difference in nucleotide or protein sequence between one or more individuals or one or more cells within an individual (e.g. in cancer cells within an individual). A mutation that results in a difference in an amino acid may also be called an amino acid substitution mutation.

In some examples of the disclosed method, the mutation is a point mutation in the silencing domain of a GW182 polypeptide that results in an amino acid substitution that renders the polypeptide dominant negative. In other examples, the mutation is a deletion of one or more amino acids in the silencing domain of a GW182 polypeptide—up to and including a deletion of the entire silencing domain—that renders the polypeptide dominant-negative (up to 550 or more amino acids). In other examples, the mutation is a deletion of at least 100 amino acids within the silencing domain, including the final 100 amino acids or any 100 amino acid deletion within the silencing domain that renders the polypeptide dominant negative. In other examples the mutation is a deletion of 50 or fewer amino acids in the silencing domain that renders the polypeptide dominant negative. Examples of polypeptides that may be used in the disclosed method include any 50 or fewer amino acid, 50-100 amino acid, or 100-550 amino acid deletion in (or of) the silencing domain of any GW182 polypeptide from any species, including any of SEQ ID NO: 01-07 or any deletion of less than 50 amino acids, 50-100 amino acids, or 100-550 amino acids from the C-terminus of any of SEQ ID NO: 01-07 that renders the protein dominant negative or any polypeptide that is at least 50%, at least 60%, at least 70%, at least 80%, at least 90%, at least 95%, at least 99% or about 100% homologous to any such mutation. Further examples of polypeptides that may be used in the disclosed method include polypeptides that are at least 50%, at least 60%, at least 70%, at least 80%, at least 90%, at least 95%, at least 99%, or about 100% homologous to SEQ ID NOs 08-11.

A domain of a polypeptide or protein may be any part of a protein that can be demonstrate to mediate a particular protein function. For example, the silencing domain of Drosophila GW182 may be defined as running from amino acid 861 to the C terminus of SEQ ID NO: 01. The silencing domain may be mutated by any of a number of methods known in the art such as site directed mutagenesis or deletion of part or all of the silencing domain by restriction digestion using natural or artificially engineered restriction sites. Identification any GW182 polypeptide, identification of the silencing domain of any GW182 polypeptide, selection or engineering of a mutation in the silencing domain, and confirmation of the ability of the deletion to render the polypeptide dominant negative will be readily available to one skilled in the art in light of this disclosure without undue experimentation.

The precise number of amino acids making up the silencing domain varies depending on the species of eukaryote from which the GW182 polypeptide was derived, as well as the isoform of the GW182 polypeptide within a species. Rather than a precise structural definition based on the number of amino acids, it is the maintenance of dominant negative function that is important when selecting the amino acid sequence of particular polypeptide to be used in the disclosed method.

A mutation may also occur in a microRNA including a microRNA of interest. A microRNA mutation may result in greater or lesser binding and/or silencing of a target mRNA or may result in a different profile of mRNA regulation by the microRNA of interest. A mutated microRNA may be naturally occurring or made by humans.

Contacting a molecular entity such as a microRNA of interest with another molecular entity such as a polypeptide encompasses placement of the two molecular entities in direct physical association. Physical associations may involve the mixing of solid (including particulate solids), liquid, and gaseous molecular entities in close proximity such as solid with solid, solid with liquid, liquid with liquid, liquid with gas, etc. Contacting includes the addition of one liquid to another liquid. Contacting also includes the placement of one or more molecules in the same space such as transfecting a polynucleotide into a cell. Additionally, contacting includes mixing of components in a cell-free system or in cells that have been lysed without transfection.

In some examples of the disclosed method, contacting the microRNA of interest with the dominant negative GW182 polypeptide involves transfecting the cell with a nucleic acid construct. In further examples, the nucleic acid construct comprises a polynucleotide sequence comprising the sequence of the microRNA of interest. In some further examples of the disclosed methods, the nucleic acid construct comprises the pre-microRNA of the microRNA of interest. The pre-microRNA may assume a stem-loop structure. In this example, the construct may comprise only the pre-microRNA sequence of the microRNA of interest and no other sequence. In other examples, the construct may comprise only the mature microRNA sequence of the microRNA of interest. In still further examples, the nucleic acid construct further comprises a second polynucleotide sequence that further comprises a promoter operably linked to the sequence of the microRNA of interest, wherein the microRNA of interest is expressed and potentially overexpressed in the cell.

In some embodiments of the invention, the cell is transfected with a nucleic acid construct that comprises a nucleotide sequence that encodes a dominant negative GW182 polypeptide. For example, the nucleic acid construct may comprise SEQ ID NO: 12, wherein SEQ ID NO: 12 comprises a mutation that renders the polypeptide that it encodes dominant negative. The sequence encoding the dominant negative polypeptide may be at least 50%, at least 60%, at least 70%, at least 80%, at least 90%, at least 95%, at least 99%, or about 100% homologous to any dominant negative version of SEQ ID NO: 12. Examples of nucleic acid sequences that encode dominant negative GW182 polypeptides include SEQ ID NOs: 13-15 and any sequence that is at least 50%, at least 60%, at least 70%, at least 80%, at least 90%, at least 95%, at least 99%, and about 100% homologous to any of those sequences.

One indication that two nucleic acid molecules are closely related is that a nucleic acid molecule will hybridize to the complement of its related nucleic acid molecule under stringent conditions. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to a nucleic acid that encodes a protein can be determined by this method.

A promoter may be any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of skill in the art. Examples include tissue specific promoters that predominantly transcribe genes in the context of a cell of a particular type or lineage (such as a lymphoid cell, a neuronal cell, a muscle cell, etc.) Other examples include inducible promoters that predominantly transcribe genes in the presence or absence of a particular drug, nutrient, or other compound.

A first nucleic acid sequence is said to be operably linked with a second nucleic acid sequence when the first nucleic acid sequence is placed in such a way that it may have an effect upon the second nucleic acid sequence. For instance, a promoter is operably linked to a coding sequence if the promoter affects the transcription or expression of the coding sequence. Operably linked DNA sequences may be contiguous, or they may operate at a distance. Where necessary to join two protein coding regions, operably linked DNA sequences are both contiguous and in the same reading frame.

Transfection may be any method of introducing polynucleotides into a cell. Many methods of transfection involve the transient creation of pores within the cell membrane. Polynucleotides or other molecules then enter the cell through the pores via diffusion. The pores then close, preferably leaving the cell otherwise unaffected. Transfection may be carried out through any of a number of methods. Some such methods involve the use of chemicals such as calcium phosphate, cyclodextrin, dendrimers, liposomes, cationic polymers (such as DEAE dextran or polyethylenimine), and/or any of a number of proprietary transfection agents known in the art (e.g., Lipofectamine® transfection agent) or yet to be disclosed.

Transfection may also be carried out through non-chemical methods. These may include electroporation, sonication, optical transfection, or any other method using electricity, magnetism, or another physical force to cause nucleic acid to enter a cell. Additionally, transfection may be performed using particle based methods. Examples of such methods include the gene gun, in which nanoparticles of an inert solid (such as gold) are physically propelled into the nucleus of a cell. Magnetofection involves propelling nucleic acids associated with magnetic nanoparticles associated with DNA into cells. Other particle based methods are now known in the art or yet to be disclosed.

For a nucleic acid to be stably transfected, it must be integrated into the genome of the cell so that it is replicated during mitosis. To generate a stably transfected cell line, a nucleic acid construct comprising the gene of interest is transfected into the cell by any transfection mechanism. The nucleic acid construct can comprises a marker gene that allows selection of the stable transfectants. Often the marker gene involves resistance to a particular drug such that when the drug is introduced into the cell culture, the only cells that survive are those that have integrated the nucleic acid construct into their genomes. Prior to treatment with the drug, cells may be cloned by limiting dilution into single cell cultures. A fluorescent or bioluminescent gene such as GFP or luciferase may also be used as a marker gene. Cells that carry the marker gene are confirmed to also carry the gene of interest.

Another method of introducing nucleic acid into a cell is viral transduction. In this process, DNA is introduced into the cell via a viral vector. Viruses naturally infect cells with viral nucleic acids which are then translated into viral protein via cellular machinery. Viruses may be also engineered to infect a cell with a polynucleotide that comprises a sequence that encodes a protein of interest, resulting in the translation and expression of the protein of interest.

One example of such a virus that is used in mammalian cells is adenovirus. Adenoviruses infect a wide range of cell types, including both replicating and non-replicating cells. In some examples of adenoviral transduction systems, the viral E1 early genes are removed, rendering the virus unable to replicate within the cells. If such a mutant form of a virus is used to infect the cell, then a gene of interest (operably linked to an appropriate promoter) cloned into the viral vector can be introduced within the cell and readily expressed. Should contacting the microRNA of interest with the polypeptide involve the use of viral transduction, then in some further examples, the nucleic acid construct that encodes the dominant negative GW182 polypeptide may further comprise adenovirus genes. Such a construct may further comprise a promoter such as a cytomegalovirus (CMV) promoter. Examples of such a construct include SEQ ID NOs: 16-18.

Another example of a virus that may be used in viral transduction is a retrovirus. A retrovirus is an RNA virus that uses viral reverse transcriptase to produce a cDNA from its RNA transcript. The cDNA is then incorporated into the cellular genome using viral integrase. This allows delivery of a nucleic acid construct into a cell and integration of the construct into the genomic DNA of the cell. There are many types of retroviruses, of which lentiviruses (such as HIV, SIV, and FLV) are but one type.

Contacting the microRNA of interest with the dominant negative GW182 polypeptide within a cell may occur in any combination. In one combination, a single construct that expresses both the microRNA of interest and the dominant negative GW182 polypeptide may be transfected into the cell. In another combination, the microRNA of interest is transfected into a cell stably transfected with a construct that expresses the dominant negative GW182 polypeptide. In another combination, a construct that expresses the dominant negative GW182 polypeptide is transfected into a cell stably transfected with a construct that expresses the microRNA of interest. In another combination, a construct that expresses the dominant negative GW182 polypeptide is cotransfected with a separate construct comprising the microRNA of interest. In another combination, both the construct that expresses the dominant negative GW182 polypeptide and a construct that expresses the microRNA of interest are stably transfected into the same cell line. Contacting a microRNA of interest with a polypeptide encompasses any way to bring together a microRNA and a polypeptide within a cell now known in the art and yet to be disclosed.

In some examples of the disclosed method, the method further comprises lysing the cell. Cellular lysis may be any viral, enzymatic, osmotic, or other mechanism that results in a complete loss of cellular integrity, generally characterized by release of cytoplasmic and other components. Lysis of the cell may occur before or after contacting of the microRNA of interest with the polypeptide. Lysis may also occur during transfection, but lysis as a result of transfection would not be a preferred embodiment of the method. In further examples, lysis is performed after transfection but prior to the purification of the complex comprising the dominant negative GW182 polypeptide and the target mRNA.

Some examples of the disclosed method involve purification of the complex comprising the dominant negative GW182 polypeptide and the target mRNA. Purification of the complex may be achieved by any method now known or yet to be disclosed. In some examples, purification is achieved by contacting the complex with a first reagent capable of binding to a component of the complex to a component of the complex to the exclusion of other cellular components. The first reagent may bind any possible component of the complex, including the dominant negative GW182 polypeptide, the target mRNA, the microRNA, or any other component of the complex such as one or more Argonaute (Ago) proteins such as proteins with SEQ ID NO: 29 or SEQ ID NO: 30. In some examples, the first reagent comprises an antibody that binds to one or more components of the complex.

A reagent capable of specific binding to a biomolecule may be any reagent that associates preferably (in whole or in part) with a particular biomolecule. A reagent binds specifically when it binds predominantly to a defined target. It is recognized that a minor degree of non-specific interaction may occur between a molecule, such as a specific binding reagent and an off-target biomolecule. Nevertheless, specific binding can be distinguished as mediated through specific recognition of the biomolecule by the reagent.

Specific binding reagents typically bind to a polypeptide with a more than 2-fold, such as more than 5-fold, more than 10-fold, more than 100-fold, or more than 10.000-fold greater amount of bound reagent (per unit time) to the polypeptide compared with the reagent's binding to a non-target (negative control) polypeptide. Specific binding may also be determined by a binding affinity calculation. Methods for performing such calculations are well known in the art. Specific binding results in binding affinity values calculated as [BR][T]/[BR·T] wherein BR=binding reagent and T=the target of the binding reagent on the order of 10⁻⁴, 10⁻⁵, 10⁻⁶, 10⁻⁷, 10⁻⁸, 10⁻⁹, 10¹⁰ or lower. Other examples of specific binding reagents include natural ligands, engineered nanoparticles, or any other reagent capable of specific binding.

An antibody may be any polypeptide that includes at least a light chain or heavy chain immunoglobulin variable region and specifically binds an epitope of an antigen. Antibodies can include monoclonal antibodies, polyclonal antibodies, or fragments of antibodies. A variety of assay formats are appropriate for selecting antibodies specifically immunoreactive with a particular biomolecule. For example, solid-phase ELISA immunoassays are routinely used to select monoclonal antibodies specifically immunoreactive with a protein. In some examples of the invention, the reagent comprises an antibody capable of specific binding to the polypeptide or another polypeptide that is a member of the complex.

In other examples of the disclosed method, the polypeptide may comprise a label and the reagent is capable of specific binding to the label. A label may be any substance capable of aiding a machine, detector, sensor, device, column, or enhanced or unenhanced human eye from differentiating a labeled composition from an unlabeled composition. Labels may be used for any of a number of purposes and one skilled in the art will understand how to match the proper label with the proper purpose. Examples of uses of labels include purification of biomolecules, identification of biomolecules, detection of the presence of biomolecules and localization of biomolecules within a cell, tissue, or organism. Examples of labels include but are not limited to: radioactive isotopes or chelates thereof; dyes (fluorescent or nonfluorescent); stains; enzymes; nonradioactive metals; magnets, such as magnetic beads; protein tags; any antibody epitope; any specific example of any of these; any combination between any of these; or any label now known or yet to be disclosed.

A label may be covalently attached to a biomolecule or bound through hydrogen bonding, Van Der Waals or other forces. A label may be associated with the N-terminus, the C-terminus or any amino acid in the case of a polypeptide or the 5′ end, the 3′ end or any nucleic acid residue in the case of a polynucleotide. Examples of a dominant negative GW182 polypeptide comprising a label include dominant negative TNRC6A, TNRC6B, TNRC6C or any isoform thereof bound to a label. One example of such a label comprises biotin, which facilitates purification of the labeled polypeptide through the interaction of the biotin label with streptavidin, avidin, any other biotin binding molecule. Examples of nucleic acid constructs encoding such a polypeptide include SEQ ID NO: 19, SEQ ID NO: 20, and SEQ ID NO: 21.

One type of label is a protein tag. A protein tag comprises a sequence of one or more amino acids that may be used as a label as discussed above. In some examples, the protein tag is covalently bound to the polypeptide. It may be covalently bound to the N-terminal amino acid of the polypeptide, the C-terminal amino acid of the polypeptide or any other amino acid of the polypeptide. Often, the peptide tag is encoded by a polynucleotide sequence that is immediately 5′ of a nucleic acid sequence coding for the polypeptide such that the protein tag is in the same reading frame as the nucleic acid sequence encoding the polypeptide. Protein tags may be used for all of the same purposes as labels listed above and are well known in the art. Examples of protein tags include chitin binding protein (CBP), maltose binding protein (MBP), glutathione-S-transferase (GST), poly-histidine (His), thioredoxin (TRX), FLAG, V5, c-Myc, HA-tag, green fluorescent protein (GFP) modified GFPs and GFP derivatives and other fluorescent proteins, such as EGFP, EBFP, YFP, BFP, CFP, ECFP and so forth. Other tags include a His-tag which facilitates purification on metal matrices. Other protein tags include BCCP, calmodulin, Nus, Thioredoxin, Strep, SBP, and Ty, or any other combination of one or more amino acids that aids in the purification of biomolecules, the identification of biomolecules, the detection of the presence of biomolecules, or the localization of biomolecules within a cell, tissue, or organism.

Examples of a dominant negative GW182 polypeptide with a protein tag include dominant negative TNRC6A, TNRC6B, TNRC6C or any isoform thereof coupled to a protein tag. Examples of protein tags that may be used include myc, GFP, and FLAG-HA.

Examples of nucleic acids encoding such polypeptides include SEQ ID NOs: 22-28. In examples of the method in which the polypeptide comprises a protein tag, the method may include purifying the complex with a reagent that specifically binds to the protein tag.

In some examples of the disclosed method, the complex comprising the dominant negative GW182 polypeptide and the target mRNA comprises an additional polypeptide. In further examples, the additional polypeptide binds the dominant negative GW182, but it may also bind the target mRNA, the mRNA of interest, or some combination of these and the dominant negative GW182. Alternatively, the additional polypeptide binds to none of these, but does bind to another component of the complex. In these examples, the complex may be purified by a reagent that specifically binds to the additional polypeptide or to a label (such as a protein tag) bound to the additional polypeptide as described above. In the case where the first polypeptide is a dominant negative GW182 polypeptide, the additional polypeptide may be a member of the Ago family, such as mammalian Ago-1, Ago-2, Ago-3, Ago-4, Ago-5, Ago-6, Ago-7 or any member of the Ago family now known or yet to be disclosed or a protein with 50%, 60%, 75%, 80%, 90%, 99% or 100% homology to one or more members of the Ago family (such as SEQ ID NO: 29 and SEQ ID NO: 30). In further examples, the reagent used to purify the complex is an antibody.

In some examples of the disclosed method, the target mRNA is identified. In general, a target mRNA is identified on the basis of all or part of its nucleic acid sequence. Any method of detecting a particular nucleic acid sequence of an mRNA module known in the art or yet to be developed may be used to identify the sequence of the target mRNA. Once the sequence of the target mRNA is identified, it may be compared to a database such as GenBank in order to identify it as a particular previously discovered mRNA sequence or it may comprise an mRNA sequence that has yet to be recorded. Identification of the target mRNA may also allow identification of the protein encoding the target mRNA.

Some methods of identifying the target mRNA comprise mass spectrometry. Mass spectrometry may be any method by which a sample is analyzed by generating gas phase ions from the sample. Such ions are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography or gel-electrophoretic separation. Mass spectrometry may be performed on the complex or target mRNA purified from the complex. Mass spectrometry may be especially useful (though not necessary) if the effects of multiple microRNA of interest are used to generate a combined microRNA profile.

Other methods of identifying the target mRNA include methods that involve binding of the target mRNA to a reagent capable of specific binding to a nucleic acid sequence similar to all or part of the target mRNA or to a nucleic acid sequence conjugated to the target mRNA, including a nucleic acid sequence added to the target mRNA by recombinant DNA technology. One example of such a reagent is an oligonucleotide. An oligonucleotide may be any nucleic acid of two or more nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 300 nucleotides in length, for example a nucleic acid sequence (such as DNA or RNA) that is at least 6 nucleotides, for example at least 8, at least 10, at least 15, at least 20, at least 21, at least 25, at least 30, at least 35, at least 40, at least 45, at least 50, at least 60, at least 70, at least 80, at least 90, at least 100 or even at least 120, at least 150, or at least 200 or more nucleotides long, or from about 6 to about 50 nucleotides, for example about 10 to 25 nucleotides, such as 12, 15 or 20 nucleotides. An oligonucleotide probe may be at least 8, at least 10, at least 15, at least 20, at least 21, at least 25, at least 30, at least 45, at least 60, at least 70, at least 100 or even at least 120, at least 150, or at least 200 nucleotides in length that is used to detect the presence of a complementary sequence by molecular hybridization. In particular examples, oligonucleotide probes include a label that permits detection of oligonucleotide hybridization complexes comprising the probe and the target sequence of the probe.

In examples of the disclosed method in which the reagent that binds the target mRNA is an oligonucleotide, the reagent may be bound to a solid phase support. Well-known supports or carriers include glass, silicone dioxide or other silanes, polyvinyl, polystyrene, polypropylene, polyethylene, dextran, nylon, amylases, natural and modified celluloses, polyacrylamides, hydrogels, gold, platinum, microbeads, micelles and other lipid formations, and magnetite. The oligonucleotide reagent may be affixed, attached, or printed onto the substrate either singly or with a plurality of similar or different oligonucleotide reagents in the format of a microarray. In examples of the method in which the third reagent is bound to a microarray, the target mRNA may be identified on the basis of binding of the target mRNA to the reagent. Using a microarray, a target mRNA profile of a microRNA of interest in an experimental condition may be generated. The solid support may be constructed in any physical form appropriate for a given type of analysis. For example, it may be constructed as a flat surface as in the case of a microarray or it may be constructed as a bead or other shape.

In examples of the disclosed method in which the reagent that binds to the target mRNA is an oligonucleotide, the reagent may be used as a primer or probe to be used in a reverse transcription reaction, a nucleic acid amplification reaction, a nucleic acid sequencing reaction, or any other method in which an oligonucleotide may be used in the identification of a target mRNA. Note that in light of this disclosure, one of skill in the art will understand how to generate a primer or probe that may be used to identify a target mRNA using any of these techniques. Note also that an oligonucleotide used in the identification of a target mRNA may be a degenerate oligonucleotide. A degenerate oligonucleotide is an oligonucleotide intended to bind to and/or amplify a plurality of nucleic acid sequences including nucleic acids with unknown sequences. Such oligonucleotides have variable bases at certain positions and/or target highly conserved regions of mRNA. Those of skill in the art will understand how to construct a degenerate oligonucleotide that may be used as a primer or probe or any other component of a technique used in the identification of a target mRNA.

Identifying the target mRNA may comprise performing a reverse transcription reaction of the target mRNA. Reverse transcription of mRNA may be performed using a reverse transcriptase such as avian myeloblastosis virus reverse transcriptase (AMV-RT) or Moloney murine leukemia virus reverse transcriptase (MMLV-RT). Reverse transcription is primed using any of a number of primers including an oligonucleotide primer with specificity to the target mRNA, a degenerate oligonucleotide primer, random hexamers, or an oligo-dT primer. A primer with specificity to a target mRNA would likely be suboptimal when identifying an unknown target mRNA of a microRNA of interest. Reverse transcription of mRNA results in a cDNA product with a sequence that is identical to the mRNA except that uracil (U) nucleotides in the mRNA are replaced with thymine (T) nucleotides in the cDNA.

The product of a reverse transcription reaction can be amplified by any of a number of methods. In general, nucleic acid amplification is a process by which copies of a nucleic acid may be made from a source nucleic acid. In some nucleic amplification methods, the copies are generated exponentially. Examples of nucleic acid amplification include but are not limited to: the polymerase chain reaction (PCR), ligase chain reaction (LCR,) self-sustained sequence replication (3SR), nucleic acid sequence based amplification (NASBA,) strand displacement amplification (SDA,) amplification with Q replicase, whole genome amplification with enzymes such as φ29, whole genome PCR, in vitro transcription with Klenow or any other RNA polymerase, or any other method by which copies of a desired sequence are generated.

Polymerase chain reaction (PCR) is a particular method of amplifying DNA, generally involving the making of a reaction mixture by mixing a nucleic sample, two or more primers, a DNA polymerase, which may be a thermostable DNA polymerase and deoxyribose nucleoside triphosphates (dNTP's). In general, the reaction mixture is subjected to temperature cycles comprising a denaturation stage (typically 80-100° C.) an annealing stage with a temperature that may be based on the melting temperature (Tₘ) of the primers and the degeneracy of the primers, and an extension stage (for example 40-75° C.) The Tₘ of the primers may be calculated by any of a number of methods known in the art, including any software that estimates Tₘ on the basis of oligonucleotide sequence.

Quantitative PCR and/or real-time PCR is a method of measuring the amount of nucleic acid template present in an original mixture by correlating the speed of amplification of the specific PCR product with the amount of nucleic acid template originally present in the mixture. When used to identify target mRNA, it may also be used to quantify the amount of the target mRNA bound by the microRNA of interest. When performed on a reverse transcription product, quantitative PCR may also be referred to as quantitative reverse transcription PCR. One example of quantitative PCR is the TAQMAN® system. In this example, two oligonucleotide primers are used to generate an amplicon typical of a PCR reaction. A third oligonucleotide, or probe, is designed to detect nucleotide sequence located between the two PCR primers. The probe is nonextendable by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data. Examples of fluorescent labels that may be used in quantitative PCR include but need not be limited to: HEX, TET, 6-FAM, JOE, Cy3, Cy5, ROX TAMRA, and Texas Red. Examples of quenchers that may be used in quantitative PCR include, but need not be limited to TAMRA (which may be used as a quencher with HEX, TET, or 6-FAM), BHQ1, BHQ2, or DABCYL.

TAQMAN® RT-PCR can be performed using commercially available equipment, such as, for example, ABI PRISM 7700® Sequence Detection System™ (Perkin-Elmer-Applied Biosystems, Foster City, Calif., USA), or Lightcycler (Roche Molecular Biochemicals, Mannheim, Germany). Any real-time PCR system may include one or more of a thermocycler, a laser, a charge-coupled device (CCD), a camera and a computer. Samples are amplified in a 96-, 384-, 1536- (or more) well format in the thermocycler. During amplification, a laser-induced fluorescent signal is collected in real time through fiber optic cables for all wells, and detected at the CCD. The system includes software for running the instrument and for analyzing the data. In some examples, assay data are initially expressed as Ct (cycle threshold). Fluorescence values are recorded during every cycle and represent the amount of product amplified to that point in the amplification reaction. To minimize errors and the effect of sample-to-sample variation, RT-PCR can be performed using an internal standard.

Additionally, quantitative PCR may be performed upon a cDNA resulting from the reverse transcription of a sample from a subject without the use of a labeled oligonucleotide probe that binds to a sequence between the primers. In some of these techniques, PCR amplification is tracked by the binding of a fluorescent dye such as SYBR green to the double stranded PCR product during the amplification reaction. SYBR green binds to double stranded DNA, but not to single stranded DNA. In addition, SYBR green fluoresces strongly at a wavelength of 497 nm when it is bound to double stranded DNA, but does not fluoresce when it is not bound to double stranded DNA. As a result, the intensity of fluorescence at 497 nm may be correlated with the amount of amplification product present at any time during the reaction. The rate of amplification may in turn be correlated with the amount of template sequence present in the initial sample. Generally, Ct values are calculated similarly to those calculated using the TaqMan® system. Because the probe is absent, amplification of the proper sequence may be checked by any of a number of techniques. One such technique involves running the amplification products on an agarose or other gel appropriate for resolving nucleic acid fragments and comparing the amplification products from the quantitative real time PCR reaction with control DNA fragments of known size.

Note that identifying a nucleic acid through the use of PCR need not involve real-time PCR. Determining whether or not a specific nucleic acid molecule is present in a reverse transcription product, one need only perform PCR using oligonucleotide probes that specifically bind part of the product, perform one or more cycles of a PCR reaction, then analyze the contents of the PCR mixture by electrophoresis. Size of the product of the PCR reaction may be predicted from the locations of the selected PCR primers and compared to size standards. The sequence identity of the PCR product may be confirmed through hybridization to a labeled nucleic acid probe, through nucleic acid sequencing or any of a number of methods.

In some examples of the disclosed method, the target mRNA may be identified through the use of nucleic acid sequencing. Sequencing may be performed on cDNA or, potentially, directly on mRNA. The invention encompasses methods of identifying target mRNA through the use of DNA sequencing, such as Sanger sequencing, pyrosequencing, SOLID sequencing, massively parallel sequencing, pooled, and barcoded DNA sequencing or any other sequencing method now known or yet to be disclosed.

In Sanger Sequencing, a single-stranded DNA template, an oligonucleotide primer, a DNA polymerase, and nucleotides are used. A label, such as a radioactive label or a fluorescent label is conjugated to some of the nucleotides. One chain terminator base comprising a dideoxynucleotide (ddATP, ddGTP, ddCTP, or ddTTP, replaces the corresponding deoxynucleotide in each of four reactions. The products of the DNA polymerase reactions are electrophoresed and the sequence determined by comparing a gel with each of the four reactions. In another example of Sanger sequencing, each of the chain termination bases is labeled with a fluorescent label and each fluorescent label is of a different wavelength. This allows the polymerization reaction to be performed as a single reaction and enables greater automation of sequence reading.

In pyrosequencing, the addition of a base to a single stranded template to be sequenced by a polymerase results in the release of a pyrophosphate upon nucleotide incorporation. An ATP sulfyrlase enzyme converts pyrophosphate into ATP which in turn catalyzes the conversion of luciferin to oxyluciferin which results in the generation of visible light that is then detected by a camera.

In SOLiD® sequencing, the molecule to be sequenced is fragmented and used to prepare a population of clonal magnetic beads (in which each bead is conjugated to a plurality of copies of a single fragment) with an adaptor sequence. The beads are bound to a glass surface. Sequencing is then performed through 2-base encoding.

In massively parallel sequencing, randomly fragmented targeted DNA is attached to a surface through the use of an oligonucleotide adaptor. The fragments are extended and bridge amplified to create a flow cell with clusters, each with a plurality of copies of a single fragment sequence. The templates are sequenced by synthesizing the fragments in parallel. Bases are indicated by the release of a fluorescent dye correlating to the addition of the particular base to the fragment.

In pyrosequencing, massively parallel sequencing or SOLID sequencing, an artificial sequence called a barcode may be added to primers used to clone fragmented sequences or to adaptor sequences. A barcode is a 4-10 nucleic acid sequence that uniquely identifies a sequence as being derived from a particular sample. Barcoding of samples allows sequencing of multiple samples in a single sequencing run. (See Craig D W et al, Nat Methods 5, 887-893 (2008) for descriptions and examples of barcodes.)

In some examples of the disclosed method, the microRNA of interest is mutated relative to its native wild type form. In such examples, a microRNA profile comprising target mRNAs identified as being regulated by the mutant microRNA may be compared to the microRNA profile of the wild type microRNA.

In some examples of the disclosed method, the identification of the target mRNA is confirmed by use of another method. One example of such a method used to confirm the identification of the target mRNA comprises transfecting the microRNA of interest into a cell known to express the target mRNA and assessing the expression of the protein encoded by the target mRNA in the cell. Preferably, the cell is known to express the protein encoded by the target mRNA.

Disclosed herein are kits that facilitate the performance of the disclosed method. A kit is an assemblage of components that may be used in the performance of the method. Use of kits provides advantages to the end user of the method in that the components may have been standardized, the components may have been subject to quality assurance, the components may have been subject to sterilization, or the proportions and characteristics of the various components may have been optimized for maximal efficacy. In addition, a kit may provide the advantage that the components of the kit are obtained from a single source. This in turn makes preparations for the performance of the method as well as troubleshooting problems with the method more efficient. Components may be enclosed in one or more containers appropriate for their storage, such as vials, tubes, bottles, or any other appropriate container. The containers may be further packaged into secondary containers such as boxes, bags, or any other enclosure.

Kits used to facilitate the disclosed method may include a nucleic acid construct that encodes a dominant negative GW182 polypeptide, such as a protein with homology and/or identity to TNRC6A, TNRC6B, and TNRC6C or any isoform thereof (SEQ ID NOs 01-04) as described above. Additional components of the kit may include a reagent that can be used to purify the complex, and another reagent that facilitates the detection of the target mRNA as described above. The kit may additionally comprise a reagent that can be used in the transfection of cells such as a chemical used in transfection or an electroporation cuvette. The kit may also comprise a nucleic acid construct comprising the microRNA of interest, such as a plurality of nucleic acid sequences encoding the microRNA of interest, including a plurality of nucleic acid sequences encoding the microRNA of interest preloaded into a 96, 384, 1536, (or greater) well plate.

A kit may further comprise instructions describing how to perform the method. The instructions may be any description of the method that is provided with, referred to by, or otherwise indicated by a component of the kit. The instructions may be communicated through any tangible medium of expression. The instructions may be printed on the package material, printed on a separate piece of paper or any other substrate and provided with or separately from the kit. They may be printed in any language and may be provided in picture form. The instructions may be posted on the internet, written into a software package, or provided verbally through a telephone or by an email conversation or provided as a smart phone application. The instructions may comprise an image such as a layout of a 96 well plate. The instructions may comprise a description of the contents of a microarray. The instructions may be said to describe how to perform the method if the instructions provide a recipe of how to perform the method, if they refer a user to a publication wherein a description of the method may be found, or in any other way inform any end user of how to perform a method of identifying an target mRNA that is regulated by a microRNA of interest.

### EXAMPLES

The following examples illustrate a typical screen of endogenous mRNA targets of a microRNA of interest and are illustrative of disclosed methods. In light of this disclosure, those of skill in the art will recognize that variations of these examples and other examples of the disclosed method would be possible without undue experimentation.

### Example 1

### Methods

The following methods describe the procedures followed to produce the data described in Examples 2-17 below.

RISCtrap screens: RISCtrap screens were performed by co-transfecting 6×10⁶ HEK293T cells in a 10 cm dish with 20 μg of expression plasmid for CMV-Flag-dnGW182 and 50 nM miRNA mimics, using either Lipofectamine 2000 (Invitrogen) or a standard calcium phosphate method. Twenty-four hours post transfection, cells were rinsed with cold PBS and harvested in cold lysis buffer (20 mM Tris pH7.5, 200 mM NaCl, 1 mM DTT, 0.05% NP-40, 2.5 mM MgCl2, 60 U/mL RNAse inhibitor, and EDTA-free protease inhibitor). Cleared lysates were incubated for 2 hours at 4° C. with 104 of pre-blocked Flag-M2 agarose (2 hours with 1 mg/mL yeast tRNA and 1 mg/mL BSA). After washing the beads with lysis buffer, bound RNA was eluted by Trizol® extraction following the manufacturer's protocol (Invitrogen). Messenger RNA was enriched from 200 ng of total RNA by generation of double-stranded cDNA using an oligo-dT-T7 primer (GGCCAGTGAATTGTAATACGACTCACTATAGGGAGGCGGT₂₄-SEQ ID NO: 35) during first-strand synthesis and a standard second strand synthesis. This was followed by a 16 hour T7 IVT reaction to linearly enrich for mRNAs.

The TruSeq v2 library protocol was used on 200 ng of mRNA-enriched material from each sample to generate Illumina-compatible indexed libraries. Samples were pooled into 3 lanes and sequenced on a HiSeq v3 platform using a single-read 100 bp protocol. Reads were then uniquely mapped using TopHat v1.4.0 to a human GRCh37/hg19 reference genome and RefSeq gene annotation guidance (as of Oct. 9, 2011). A baseline of 200 counts per target across all samples was set for the dataset, non-polyadenylated transcripts were removed bioinformatically, and the dataset was normalized to the median of geometric ratios following the DESeq approach (Anders and Huber, 2010 infra). Principal component analysis was used to confirm retention of differences among conditions and clustering among biological replicates. An estimation of variance was achieved through fitting to a negative binomial distribution model (Anders and Huber, 2010 infra) and target mRNA that were significantly enriched relative to the other replicates were identified using ANOVA.

miRNA Mimics:

The design of the miRNA mimics is based on mature human sequences (miR-132: UAACAGUCUACAGCCAUGGUCG; miR-124: UAAGGCACGCGGUGAAUGCC; miR-181d: AACAUUCAUUGUUGUCGGUGGGU; miR-Scrm: AUGUGGUCCAACCGACUAAUACAG) and consists of 5′ phosphates on each strand and two-nucleotide overhangs at each 3′ end. A single basepair mismatch is introduced at nucleotide position 4 from the 3′end of the passenger strand; this intentionally designed thermodynamic instability has been demonstrated to promote efficient incorporation of the opposite guide strand (Schwarz et al., 2003, Cell.). Cel-miR-239b dsRNA oligo mimic was purchased from Dharmacon.

qPCR:

100 ng purified RNA was used for generation of first-stand cDNA using oligo-dT (mixtures of T15 and T20 oligos) following standard Superscript III® manufacturer's protocol (Invitrogen). Samples were then diluted 1:15 with water and 24 of diluted cDNA was used in triplicate 204 total reactions for qPCR with SYBR and gene-specific primers.

Flow Cytometry

Prior to flow analysis, cells were trypsinized and passed through a 35 μm mesh strainer. Flow cytometry analysis was performed on the BD Aria II®. A single 488 nm laser excited both AcGFP and DsRedEx1. Cells were gated to exclude debris and a standard doublet-exclusion was performed. Compensation was automatically calculated for each experiment using no color, AcGFP-only and DsRedEx1-only controls. AcGFP levels were detected with FL1 and a 530/30 filter, and DsRedEx1 levels were detected with FL2 and a 585/42 filter. 1×10⁴Red⁺ cells were evaluated per condition. Data was analyzed and plotted with FlowJo®.

Cell Culture, Transfections, Antibodies:

HEK293T cells were grown in DMEM media supplemented with 10% fetal bovine serum. Either Lipofectamine 2000 or calcium phosphate was used to transfect expression vectors for Flag-dnGW182, dual luciferase constructs in pSl-Check2 (Promega), CMV-Flag-PTBP1, and/or microRNA oligo mimics. Primary antibodies used for Western blotting include anti-Flag M2 (Sigma), anti-Ago1 4B8 (Sigma), anti-Ago2 (Abcam ab57113), anti-GAPDH (Millipore MAB374), CRK C-18 (SCBT), anti-HbEGF (Abcam ab16783), anti-TJAP1 (Abcam ab80444), anti-DHHC9 (Abcam ab74504), and anti-alpha-tubulin DM1A (Sigma). Secondary antibody: anti-mouse IgG-HRP or anti-rabbit IgG-HRP (Promega), anti-goat IgG-HRP (Jackson).

Definition of MREs:

MRE motifs were defined according to Baek et al, 2008 infra and Chi et al, 2012 supra. MiR-124 MREs: 8mer: gtgcctta, 7mer-m8: gtgccttN, 7mer-A1: tgcctta, 6mer: tgccttN, and pivot: gtggccttN. MiR-132 MREs: 8mer: gactgtta, 7mer-m8: gactgttN, 7mer-A1: actgtta, 6mer: actgttN, and pivots: gacctgttN or gacttgttN. MiR-181 MREs: 8mer: tgaatgta, 7mer-m8: tgaatgtN, 7mer-A1: gaatgta, 6mer: gaatgtN, and pivots: tgaaatgtN or tgagatgtN.

### Example 2

### Efficacy of Dominant-Negative TNRC6A in Stabilizing a microRNA/mRNA Complex

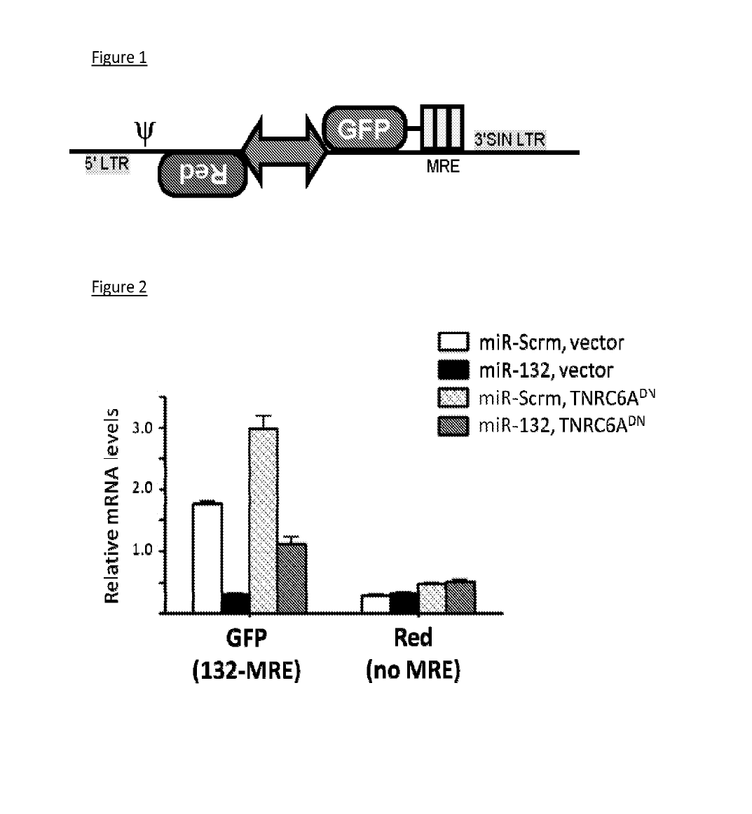

A nucleic acid construct comprising a polynucleotide sequence that encodes DsRedEx1 on one strand and a polynucleotide sequence that encodes GFP on the other strand. Both are operably linked to a bidirectional CMV promoter. The GFP is operably linked to three microRNA recognition elements (MRE's) on its 3′ end. (See top of FIG. 1 and also Magill S T, Cambronne X A et al, Proc Nat Acad Sci USA 107, 20382-20387 (2010) incorporated by reference herein.) Using this sensor construct, expression of DsRedEx1 (which fluoresces in red) is constant and can therefore be used as an internal control. Expression of the green fluorescent protein would be downregulated if a microRNA of interest binds to the MREs. The graph in FIG. 1 shows that in the presence of scrambled control microRNA mimic, the ratio of green to red protein expression from the miR-132 sensor was approximately 8:1. When miR-132 mimic was added, the green/red ratio is lower. This is consistent with a downregulation of GFP by the miR-132-RISC complex. In contrast, coexpression of a miR-132 mimic and a dominant negative TNRC6A resulted in a shift in the ratio towards the scrambled microRNA control. This indicated that the dominant-negative TNRC6A protected the mRNA from microRNA-associated silencing. To confirm that this protection was due to the stabilization of the mRNA transcript, the relative transcript levels of GFP and DsRedEx1 mRNA were measured by quantitative PCR in FIG. 2. FIG. 2 shows that expression of DsRedEx1 remained relatively constant across all experimental conditions while expression of GFP was silenced relative to the negative control by miR-132 mimic. The expression of dominant-negative TNRC6A enhanced the stability of the GFP mRNA. Note that expression of dominant-negative TNRC6A itself enhanced GFP mRNA expression. This is likely due to stabilization of the regulation conferred by endogenous miR-132 activity in these cells.

### Example 3

### Dominant Negative TNRC6a can Incorporate into an Endogenous RISC

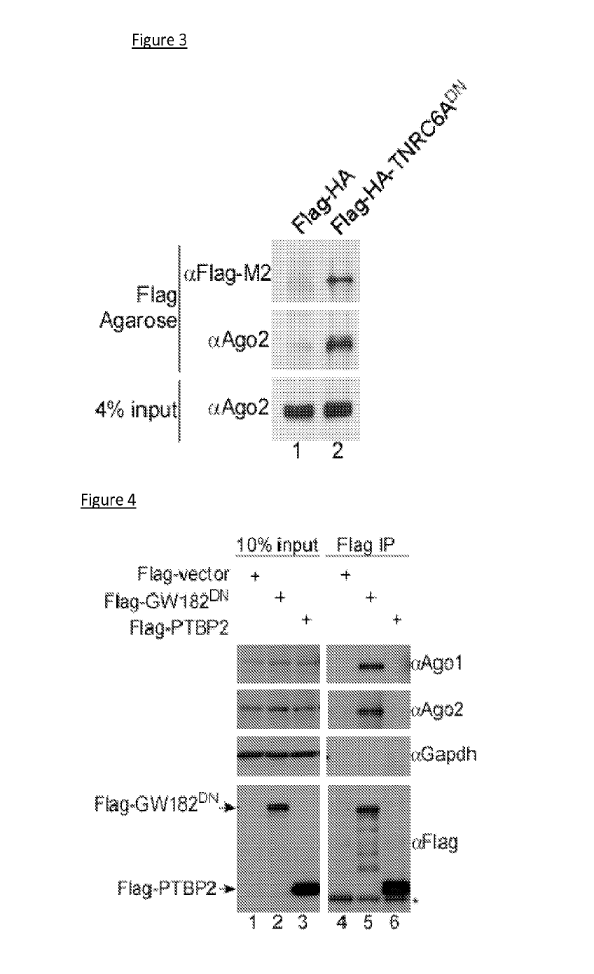

In FIG. 3, lane 2, FLAG-HA-dominant negative TNRC6A was transfected into HEK293T cells and allowed to incubate. After lysis, an immunoprecipitation was performed using anti-FLAG (top two panels). The immunoprecipitated complex was evaluated with a Western Blot and bound proteins were detected with the indicated antibodies. The Western showed that the immunoprecipitation purified a complex comprising both the dominant-negative TNRC6A and the endogenous Ago2 from the cell. Negative controls, expressing only FLAG-HA polypeptide failed to form a complex. A Western blot of the negative control detecting Ago2 demonstrated that Ago2 was present at comparable levels in both inputs. Therefore, the dominant-negative TNRC6A is capable of forming a complex with endogenous RISC complex proteins.

In FIG. 4, lanes 4-6, a FLAG immunoprecipitation was performed on HEK293T cells transfected with FLAG-vector, FLAG-dominant-negative TNRC6A (GW182^{DN}), and FLAG-PTBP2. Lane 5 shows that endogenous Ago-1 and Ago-2 each were able to form complexes with the FLAG-dominant-negative TNRC6A. No complexes were seen with empty vector or with FLAG-PTBP2. Lanes 1-3 are non-immunoprecipitated inputs. Lane 2 indicates robust expression of both dominant-negative TNRC6A and PTBP2 upon detection with an anti-FLAG antibody. Lane 5 indicates that dominant negative TNRC6A formed complexes with various types of components of endogenous RISC, resulting in a number of different RISC complexes.

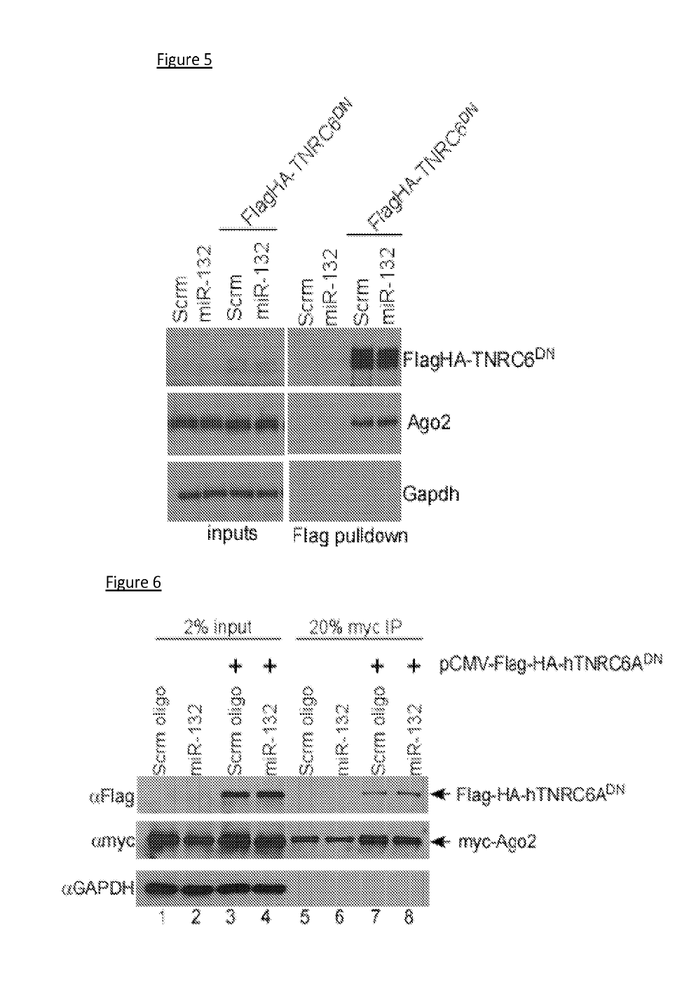

In FIG. 5, HEK293T cells coexpressed constructs in one or more of the following combinations: negative control microRNA (Scrm)+empty vector, miR-132+empty vector, negative control microRNA+dominant negative FLAG-HA-TNRC6A, and miR-132⁺ dominant negative FLAG-HA-TNRC6A. Cells were lysed. A fraction of the lysed cells was Western Blotted without any other treatment (left panels). The remainder was subjected to immunoprecipitation with anti-FLAG. FIG. 5 shows that dominant-negative TNRC6 integrated into endogenous RISC.

In FIG. 6, the same coexpression conditions were used as in FIG. 5 however, the expression was performed in a myc-Ago2 stably transfected cell line. In this case, an anti-myc immunoprecipitation was performed. Detection with an anti-FLAG antibody in a Western blot showed that the complex between dominant negative TNRC6A and Ago2 could be detected using an Ago2 immunoprecipitation. Note also that expression and immunoprecipitation with TNRC6A is clearly a better option than transfection of and immunoprecipitation with Ago2 because TNRC6A can interact with either Ago1 or Ago2 protein.

### Example 4

### Dominant Negative TNRC6a May be Used to Stabilize an Endogenous target mRNA of a microRNA of Interest

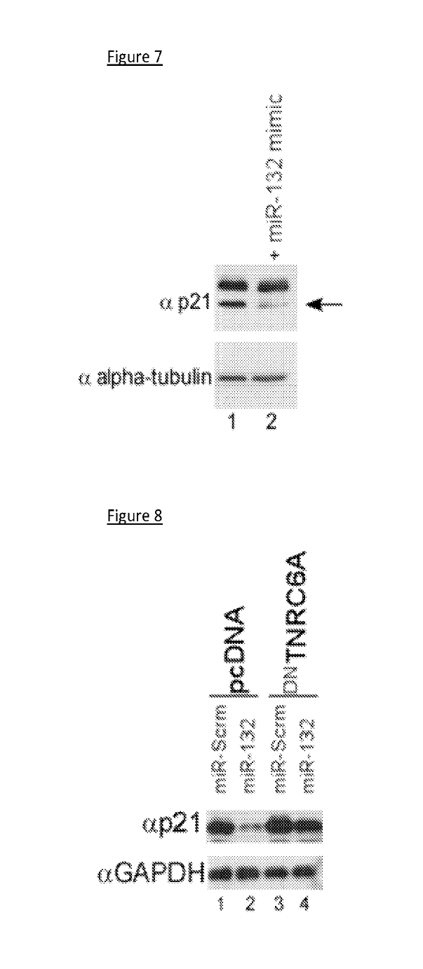

Endogenous expression of p21 in HEK293T cells is regulated by miR-132. This is demonstrated in FIG. 7. Transfection with miR-132 inhibits p21 protein expression as shown by an anti-p21 Western blot. However, FIG. 8 shows that transfection with dominant-negative TNRC6A rescues p21 protein expression from regulation by miR-132. This is hypothesized to be due to stabilization of the mRNA by the dominant negative TNRC6A.

### Example 5

### Identifying Endogenous Target mRNAs of a microRNA of Interest

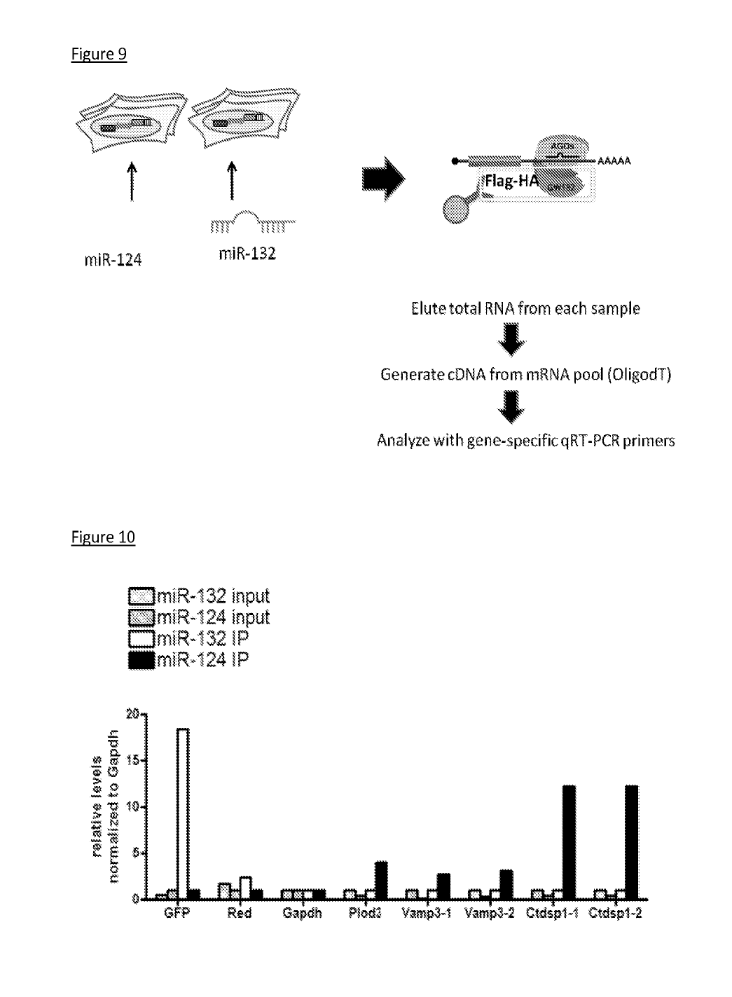

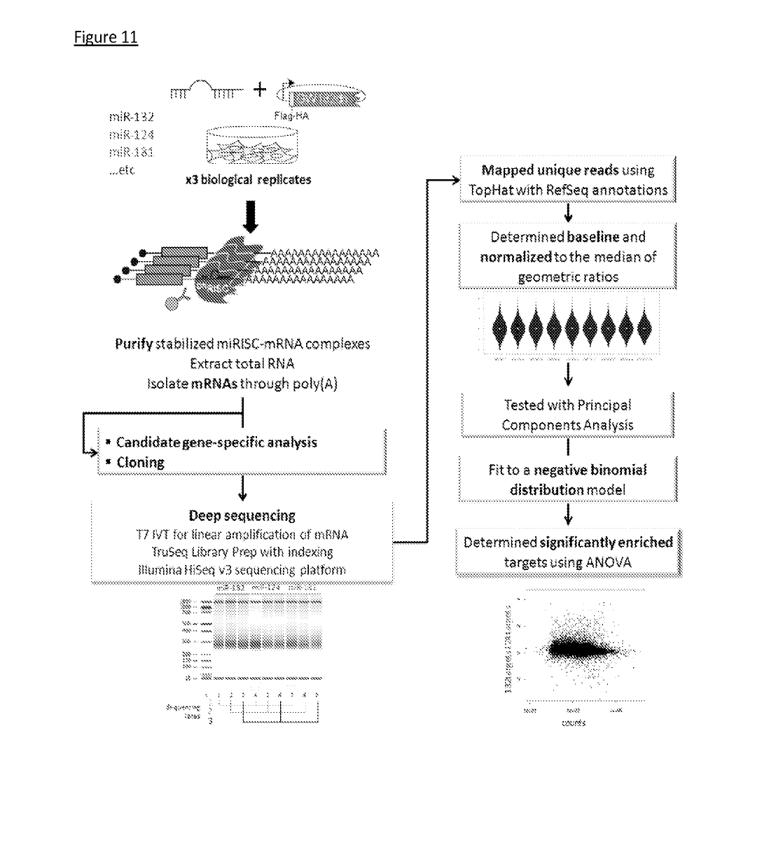

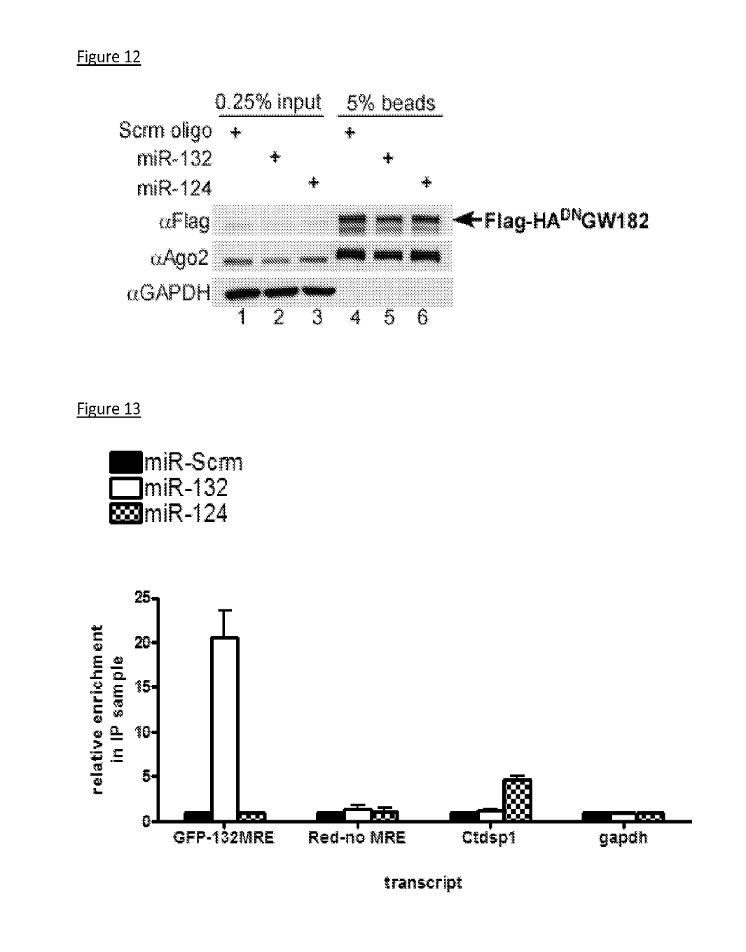

In FIG. 9, microRNA were transfected into cell lines comprising the synthetic bidirectional GFP-DsRed construct of Example 2 and dominant negative (DN) FLAG-HA-TNRC6A. A FLAG immunoprecipitation was performed and mRNA was isolated. CDNA were generated from the mRNA and the expression of the cDNA corresponding to each mRNA was assessed using quantitative PCR.

In FIG. 10, the results of the experiment described in FIG. 9 are shown. The overexpressed and exogenously added GFP mRNA was clearly detected in immunoprecipitates of FLAG-DN-TNRC6A, indicating that DN-TNRC6A could protect the GFP mRNA from miR-132 silencing in the engineered system. Additionally, endogenous target mRNA from the cells were also detected by quantitative rtPCR. In cells transfected with miR-124 and FLAG-DN-TNRC6A and immunoprecipitated with anti-FLAG, mRNA of Plod3, Vamp3, and Ctdsp1, were all identified by the assay. Vamp3 and Ctdsp1 were identified by two different sets of primer/probe sets. None of Plod3, Vamp3, or Ctdsp1 was detected in cells transfected with miR-132 and FLAG-DN-TNRC6A.

### Example 6

### Using RISC-Trap to Generate a Target mRNA Profile of a microRNA of Interest

Having demonstrated that the transfection/immunoprecipitation strategy of FIG. 9 (RISC-trap) could be used to identify endogenously expressed target mRNA of microRNA of interest, it was next demonstrated that previously unknown target mRNA of a microRNA of interest can be identified with RISC-trap. A plurality of previously known and unknown mRNA targets of a microRNA of interest would make up a target mRNA profile.

FIG. 11 illustrates a strategy to generate a target mRNA profile, which is a plurality of mRNAs that are targeted by one or more microRNA of interest under particular conditions. The box on the left corresponds roughly with the strategy in FIG. 9 using two biological replicates. The mRNA isolation and reverse transcription reaction are performed as described in Example 1 and the remaining process is performed to prepare the samples for next generation sequencing. A double stranded cDNA is produced and amplified by T7 in vitro transcription. The resulting poly-A mRNA is fragmented and a second round of reverse transcription performed to generate a first strand cDNA and second strand. Random primers, such as random 9-mers are used to perform a first-strand cDNA synthesis. A methylated dCTP may be used in first-strand DNA synthesis in combination with NotI digestion, but both of these are optional. End repair and A-tailing are both performed and appropriate next-generation DNA sequencing adaptors and barcodes are added to the ends of the fragments. The fragments are digested, size selected, and amplified by PCR. The barcoded fragments are then purified, pooled into a single sequencing reaction, and subjected to conditions that allow next generation sequencing.

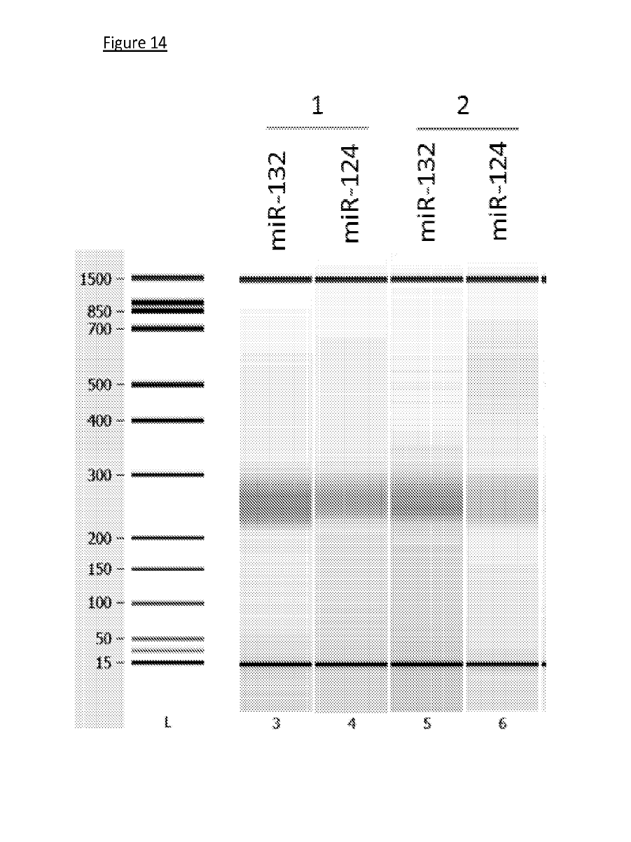

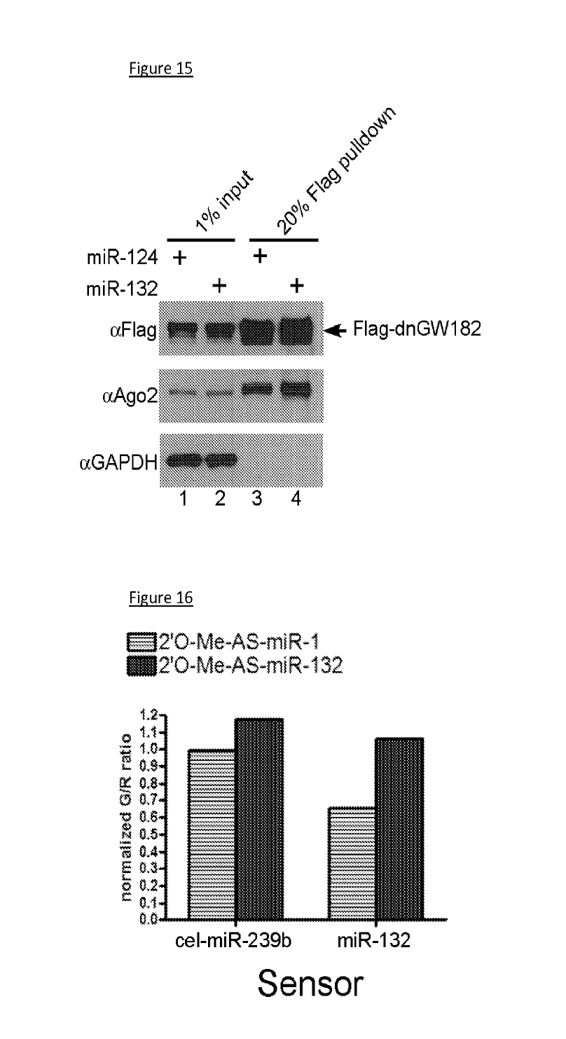

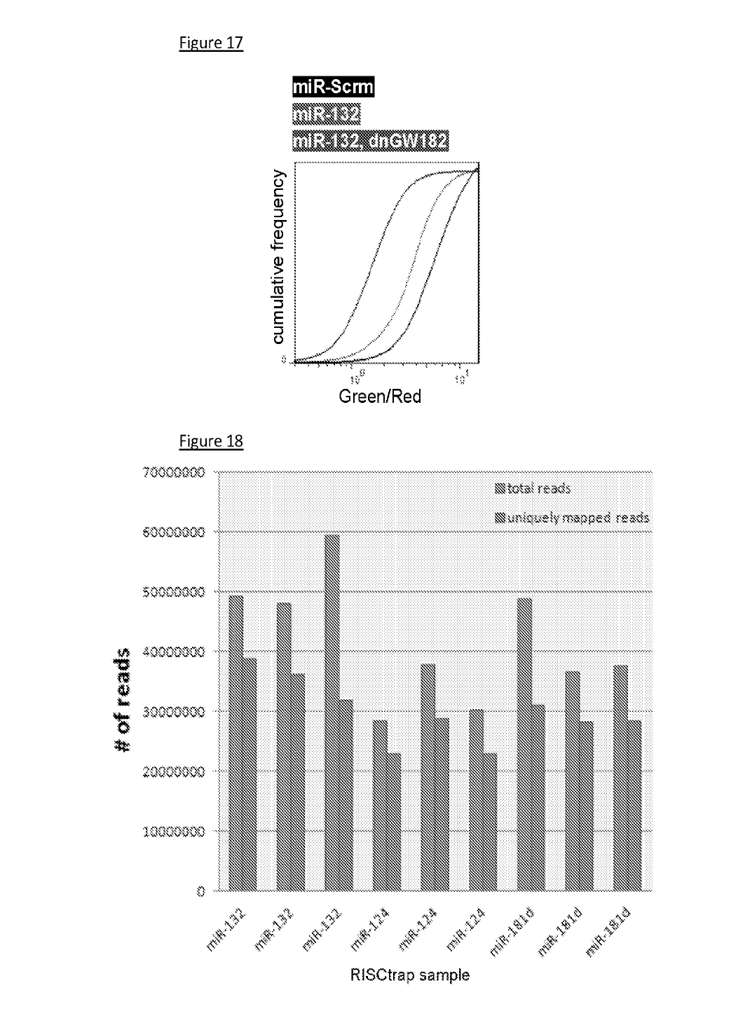

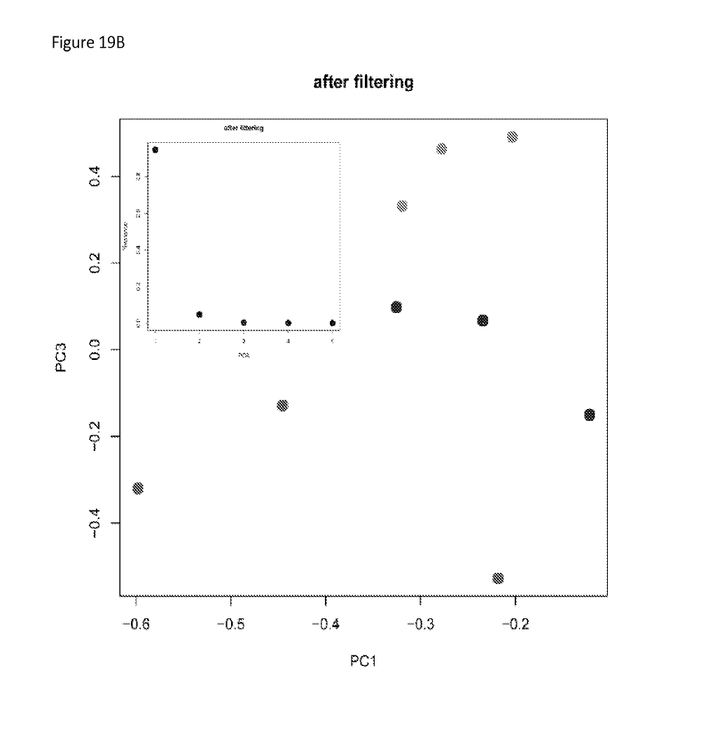

In FIG. 12, western blots of the experiment outlined in FIG. 11 are shown. Each was performed in two biological replicates. Transfection with FLAG-DN-TNRC6A and negative control, miR-132, and miR-124 was performed. Neither microRNA nor the negative control had any effect on the expression of FLAG-DN-TNRC6A or its ability to form a complex with Ago2. FIG. 17 shows that in the scale-up, GFP mRNA may be identified in cells transfected with miR-132. FIG. 13 shows that endogenously expressed Ctdsp1 is detectable as in described in Example 5. FIG. 14 shows the results from running the two biological replicates transfected with miR-132 and miR-124 on a DNA 1000 Chip run on an Agilent Bioanalyzer. Note the bands between 200- and 300 nucleotides corresponding to the fragments to be used in sequencing.

### Example 7

### Creation of New Dominant Negative GW182 Polypeptides

Amino acid and nucleotide sequences of GW182, including TNRC6 proteins from a variety of species are readily available on GenBank, NCBI, or from other sources. For example, protein and mRNA sequences of GW182 polypeptides are available through a search at www.ncbi.nlm.nih.gov/protein. Examples of GW182 polypeptides and the species from which they are derived that are available at NCBI include NCBI Reference Sequences NP_{—}055309.2 (Homo Sapiens), NP_{—}659174.3 (Mus musculus), AAX52511.1 (Drosophila melanogaster), NP_{—}001179584.1 (Bos taurus), XP_{—}003435198.1 (Canis lupus familiaris), XP_{—}001517138.2 (Orinthorhynchus anatinus), EGV97901.1 (Cricetulus griseus), XP_{—}003417280.1 (Loxodonta africana), XP_{—}003364347.1 (Equus caballus), XP_{—}003361939.1 (Sus scrofa), or any other animal, plant, or fungal homolog of GW182 now known or yet to be discovered, sequenced and/or isolated. All NCBI reference sequences are hereby incorporated by reference herein.

The silencing domain and the RRM domain are conserved across a wide number of species and may be readily identified through a BLAST search for sequence homology as described above. For example, the Drosophila melanogaster GW182, the silencing domain has been identified as amino acids 861 to about the C-terminus (amino acid 1384) (Zekri et al supra). One of skill in searching sequence databases would be able to recognize silencing domains in a GW182 protein from any species. For example, in human TNRC6A, the silencing domain commences at about amino acid 1456 and continues to about the C-terminus (amino acid 1962). In human TNRC6B (isoform 1) the silencing domain commences at about amino acid 1333 and continues to about the C-terminus (amino acid 1833). In human TNRC6C (isoform 1), the silencing domain commences at about amino acid 1211 and continues to about the C-terminus (amino acid 1725). In some examples, the silencing domain comprises the C-terminal 500-600 base pairs of the GW182 polypeptide.

Similarly, one of skill in searching sequence databases would be able to recognize the RNA Recognition Motif (RRM) in other species. In D. melanogaster, the RRM domain starts at about amino acid 1116 and continues to about amino acid 1198. In human TNRC6A (isoform 1), the RRM domain begins at about amino acid 1778 and continues to about amino acid 1862. In human TNRC6A (isoform 2) the RRM domain starts at about amino acid 1525 and continues to about amino acid 1609. IN human TNRC6B (isoform 1) the RRM domain starts at about amino acid 1535 and continues to about amino acid 1619. In human TNRC6B (isoform 2) the RRM domain starts at about amino acid 841 and continues to about amino acid 925. In human TNRC6B (isoform 3) the RRM domain starts at about amino acid 786 and continues to about amino acid 870. In human TNRC6C, the RRM domain starts at about amino acid 1511 and continues to about amino acid 1595.

Once a GW182 polypeptide is identified and its silencing domain and/or RRM domain located, dominant-negative GW182 polypeptides may be created through creating nucleic acid sequences that encode GW182 polypeptides with mutations in their silencing domains. This may be achieved through any of a number of methods. Artificial genes encoding a GW182 polypeptide comprising a point mutation, deletion, or other mutation in the silencing domain may be synthesized. Alternatively, a sequence encoding a wild type GW182 polypeptide may be PCR amplified from cDNA derived from a particular species, cloned into a plasmid or other cloning vector, and subjected to any of a number of mutagenesis methods.

Mutagenesis may be performed by any method now known or yet to be disclosed. For example, mutagenesis may involve the use of an oligonucleotide to introduce one or more point mutations in the silencing domain (such as point mutations that result in the formation of a stop codon or point mutations that alter the activity of the silencing domain.) In another example, the mutagenesis may involve the use of restriction digestion and religation to result in deletions in the silencing domain (using natural or engineered restriction sites). In another example, the mutagenesis may be a random mutagenesis introducing random mutations in the silencing domain through, for example, error prone PCR.

Once mutagenesis has been performed on the nucleic acid encoding the GW182 polypeptide, the protein may be expressed (optionally with a protein tag) and the dominant negative character of the protein confirmed. Confirmation of the dominant negative character of the resulting polypeptide may be achieved through the use of some or all of the methods described in detail in this disclosure. For example, a dominant negative GW182 would stabilize the expression of a known target mRNA in the presence of a microRNA of interest yielding a result similar to that shown in FIG. 6.

In a cell line stably transfected with the red:green sensor construct described in FIG. 1 and Example 1, a dominant negative GW182 would maintain a high green:red ratio similar to the result shown in FIG. 2 when the cell is transfected with an miRNA that regulates the MREs in the sensor construct. Alternatively, a dominant negative GW182 would also maintain expression of an endogenous protein even in the presence of a microRNA known to inhibit the expression of the protein, yielding a result like that shown in FIG. 7. These are but examples. Inhibition of microRNA silencing by any method now known or yet to be developed that indicates that a mutant GW182 polypeptide is a dominant negative GW182 polypeptide may be used by one of skill in the art to generate and confirm the dominant negative character of a dominant negative GW182 polypeptide.

### Example 8

### Assessing the Effects of Drug Treatment or Other Intervention or Condition on a microRNA Profile

A microRNA profile is a set of target mRNA that are bound by a microRNA of interest. In this example, a microRNA profile is generated according to the methods described herein. After purification of the complex comprising the dominant negative GW182 polypeptide and the target mRNA, the target mRNAs from the resulting purification are identified (for example, through sequencing, microarray, or mass spectrometry) and a list of target mRNA bound by the microRNA of interest is generated. A second set of target mRNA is then purified from the same cell line expressing a dominant negative GW182 polypeptide and comprising a microRNA of interest, but treated with a drug prior to purifying the target mRNA. Alternatively, other interventions or conditions may be combined with or substituted for the drug treatment. Such additional interventions or conditions include but are not limited to: expression of an exogenous protein, overexpression or underexpression of a tumor suppressor or tumor promoter protein, subjecting the cell line to hypoxia, increased or decreased temperature, high or low salinity or other stressor, depriving the cell line of glucose or other essential nutrients, or any other manipulation that can be performed upon a cell line.

Differences between the set of target mRNA identified in the cell line that was not treated can then be compared to the set of target mRNA identified in the cell line that was treated and the effects of the intervention on the profile of the miRNA of interest established.

### Example 9

### Assessing the Effects of a Mutant microRNA on a microRNA Profile

In this example, a microRNA profile is generated according to the methods described herein. Each target mRNA is identified and a list of target mRNA bound by the miRNA of interest is generated. A second set of target mRNA is then purified from a cell line that was transfected with a miRNA that differs from the miRNA of interest by 1-3 nucleotides and therefore represents a mutant form of the miRNA of interest. The mutant form of the miRNA of interest may be a naturally occurring mutant microRNA or an artificially engineered microRNA.

Such a microRNA profile may have any of a number of uses. It could indicate the effect of a genomic mutant form of mRNA on a cellular phenotype, or it could lead to the development of new microRNA based therapeutics, among many other uses.

### Example 10

### Description of microRNA of Interest to be Screened in the RISCtrap System

The microRNA of interest used for screening mRNA targets described in Examples 10-17 are miR-124, miR-132, and miR-181. MicroRNA-124 expression is limited to neural cells where it contributes to the differentiation of neural progenitors by targeting non-neural transcripts (Conaco C et al, Proc Natl Acad Sci USA 103, 2422-2427 (2006); incorporated by reference herein.) The direct targets of microRNA-124 have been well-studied on a global scale (Chi et al, 2009 supra; Hendrickson et al, 2008 supra; Karginov et al, 2007 supra).

MicroRNA-132 was first disclosed as an activity-dependent miRNA in excitatory neurons (Vo N et al, Curr Opin Neurobiol 20, 457-465 (2005); which is incorporated by reference herein.) In a conditional knockout mouse model, it was demonstrated that the activity of microRNA-132 is required in newborn neurons of the adult hippocampus for their proper development and survival (Magill et al, Proc Natl Acad Sci USA 107, 20382-20387 (2010), incorporated by reference herein.) Nevertheless, not as much is known about the mRNA targets of microRNA-132 and it has now been found to regulate pathways in a variety of non-neural cell types (Anand S et al, Genome Biol 11, R106 (2010); Lagos D et al, Nat Cell Biol 12, 513-519 (2010); Mellios N et al, Nat Neurosci 14, 1240-1242 (2011); Molnar V et al, Cell Mol Life Sci 69, 793-808 (2012); Shaked I et al, Immunity 31, 965-973 (2009); Taganov K D et al, Proc Natl Acad Sci USA 103 12481-12486 (2006); and Tognini P et al, Nat Neurosci 14, 1237-1239 (2011); all of which are incorporated by reference herein.)

In order to demonstrate the use of RISCtrap outside of neural cells, miR-181, which has been previously characterized in non-neural cells (Baek et al, 2008 supra; Chen C Z et al, Science 303, 83-86 (2004); Huang S et al, Nucleic Acids Res 38, 7211-7218 (2010); Iliopoulos D et al, Mol Cell 39, 493-506 (2010); and Schnall-Levin M et al, Genome Res 21, 1395-1403 (2011); all of which are incorporated by reference herein.

RISCtrap is a name of an example of a method that involves the use of a dominant negative GW182 polypeptide to identify mRNA targets of a microRNA of interest.

### Example 11

### Validation of RISCtrap

Amino acid constructs encoding dominant negative GW182 were constructed. Constructs include human TNRC6A, amino acids 1-1213, human TNRC6B, amino acids 1-1223, and human TNRC6C, amino acids 1-1215. Each dominant negative GW182 polypeptide behaved similarly in a dose-dependent and dominant manner with no additive effects. The following examples use hTNRC6A¹⁻¹²¹³ (referred to as dnGW182 below). However, any dominant negative GW182 polypeptide may be used. Dominant negative TNRC6A retains the ability to bind Argonaute but does not recruit the necessary effectors for transcript silencing and destabiliziation (Baillat D and Shiekhattar R, 2009 supra; Eulalio A et al, RNA 15, 1067-1077 (2009); Lazzaretti D et al, RNA 15, 1059-1066 (2009); and Zipprich J T et al, RNA 15, 781-793 (2009).

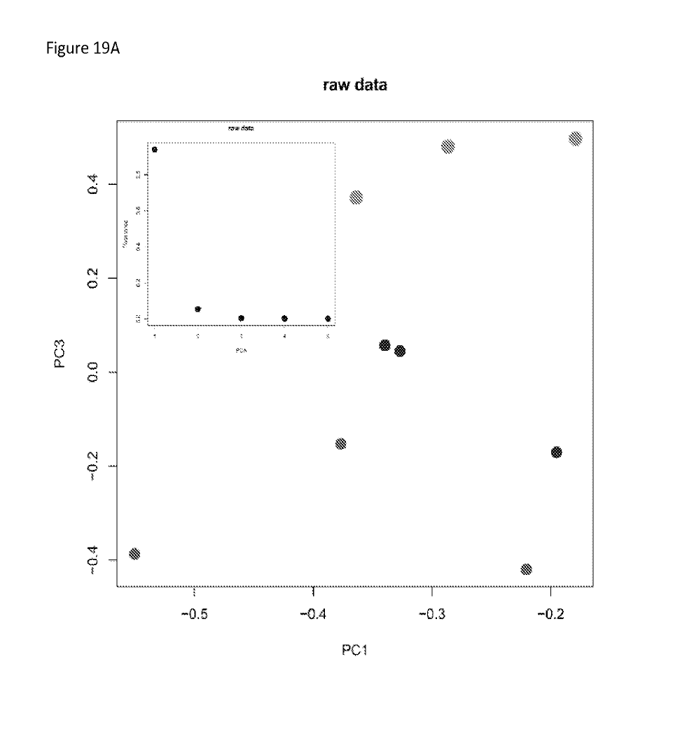

To confirm that dnGW182 properly incorporated into RISC, its ability to associate with the other RISC subunits—such as the Argonaute proteins—was established. FLAG®-tagged dnGW182 was immunoprecipitated from HEK293T cells and associated proteins were assayed by Western blot (FIG. 4). A specific interaction was detected with both endogenous Argonaute proteins 1 and 2 (Ago1 and Ago2, FIG. 4), suggesting that the RISCtrap approach can capture targets from these different versions of RISC (Landthaler Metal, RNA 14, 2580-2596 (2008), incorporated by reference herein). Moreover, dnGW182 associated with Ago2 with similar efficiencies in the presence of both miR-124 and miR-132 (FIG. 15).

DnGW182 was then used to stabilize synthetic transcripts that represented ideal positive and negative control targets for miR-132. A stable HEK293T cell line was created that constitutively expressed two synthetic transcripts co-transcribed from a bi-directional promoter (FIG. 1). As the positive control target for miR-132, one transcript encoded Green Fluorescent Protein (GFP) with three reiterated bulged miR-132 recognition elements (MRE) in its 3′ untranslated region (3′UTR). The other transcript encoded DsRedExpress1 but lacked any MREs in its 3′UTR. Co-expression of these two transcripts allowed the use of quantitative measurements, such as flow cytometry analysis, to obtain ratiometric values in individual cells that reflected miR-132 activity (Magill et al, 2010 supra). Expression of miR-132 decreased levels of the GFP transcript compared to a scrambled microRNA (miR-Scrm), without changing the abundance of the Red transcript (FIG. 2). Introduction of dnGW182 stabilized the GFP transcript in the presence of miR-132. An increase in basal GFP transcript levels upon addition of dnGW182 was also observed. This was likely due a block of endogenous miR-132 in these cells (FIG. 16).

To confirm that the stabilized GFP transcript correlated with increased GFP expression in individual cells, 10,000 cells from each condition were analyzed with flow cytometry and the cumulative frequency of the Green/Red ratio was plotted (FIG. 17). Ectopic expression of miR-132 caused a leftward shift of the plot compared to the negative control scrambled miRNA (miR-Scrm), representing a decrease of Green fluorescence compared to Red. Introduction of dnGW182 partially rescued the ratio to control levels. Together, the data demonstrated that dnGW182 could stabilize targeted transcripts.

### Example 12

### RISCtrap Identifies Known Endogenous mRNA Targets of miR124

To determine whether dnGW182 could facilitate the enrichment of targets, Flag-dnGW182 was expressed with either miR-132 or miR-124 in a cell line that constitutively expressed GFP-132 MRE and Red transcripts. Following a Flag immunoprecipitation (IP), co-enriched mRNAs were examined with qPCR. Enrichment of GFP transcript was observed in the miR-132 IP sample and not in the miR-124 IP sample (FIG. 10). In addition, miR-124 endogenous targets were enriched in the miR-124 IP sample. The Red and Gapdh transcripts, neither of which were expected to be targets of either microRNA, were not enriched in either miR-132 or miR-124 IP conditions. The enrichment of mRNA targets using RISCtrap was easily discernible over background by comparing the IP samples. There was no need to normalize to input levels as required by an Ago2 immunoprecipitation or to identify canonical MREs as required by the HITS-CLIP method.

### Example 13

### A RISCtrap Based Screen for Target mRNA of miR-124, miR-132, and miR-181

Target mRNA targets of miRNA were screened by deep sequencing (FIG. 11). The screen was performed as biological triplicates in HEK293T cells. Prior to library preparation, mRNAs were enriched from the RISCtrap purification using oligo-dT-T7 primers and a T7 in vitro transcription reaction. From each sample, 200 ng of target mRNA enriched material was used to prepare Illumina TruSeq® indexed libraries. Single-read 100 bp sequencing was performed using a HiSeq v3® platform with each biological replicate sequenced in a separate lane.

RISCtrap datasets had unique and variably-sized subsets of target transcripts with little overlap. As a result, a tailored bioinformatic approach for normalization was developed that would ensure retention of these distinct properties that likely reflected the specific targeting of each miRNA, while still being able to accurately apply statistical methods for cross-comparison of datasets. This normalization platform allowed comparison of current and future datasets from different experiments (i.e. replicates and different miRNAs). The ability of the normalization platform allows robust comparison of RISCtrap datasets obtained at different times and places, thereby obviating the need to run parallel mRNAs and increasing throughput.

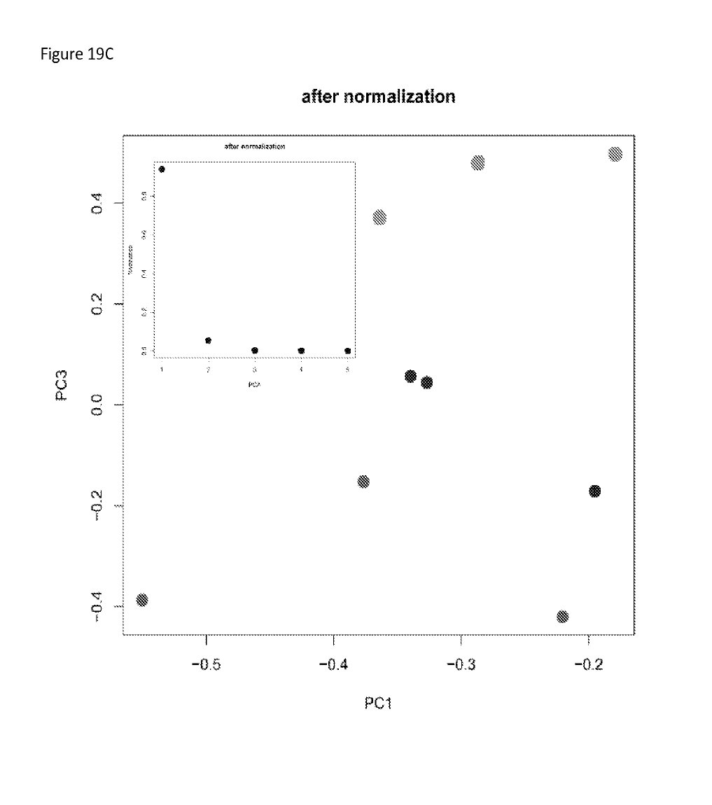

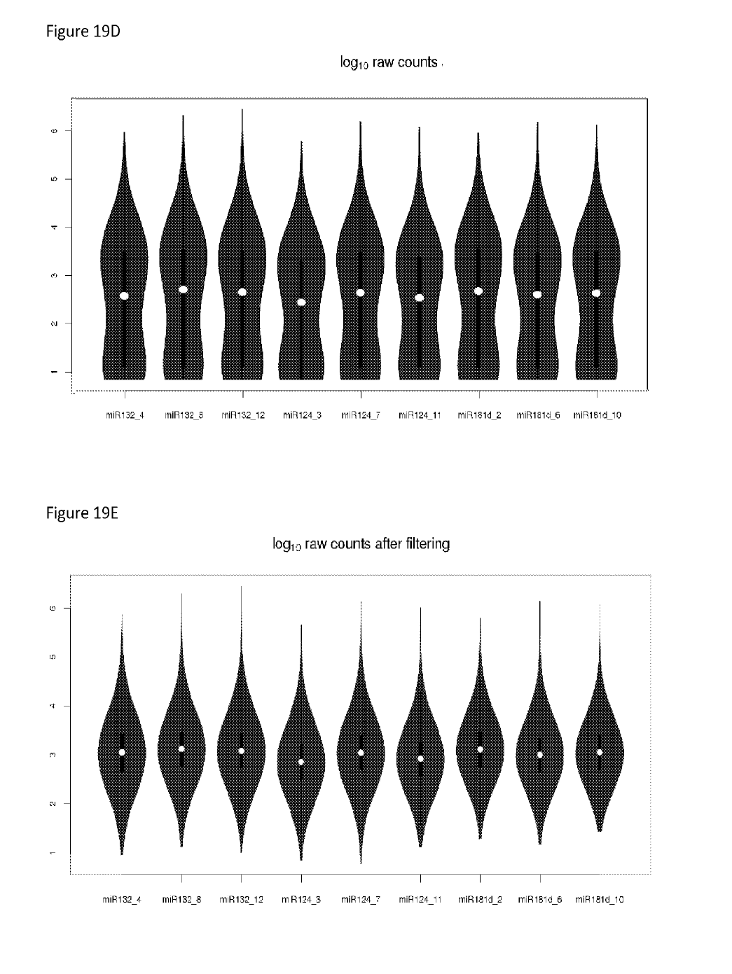

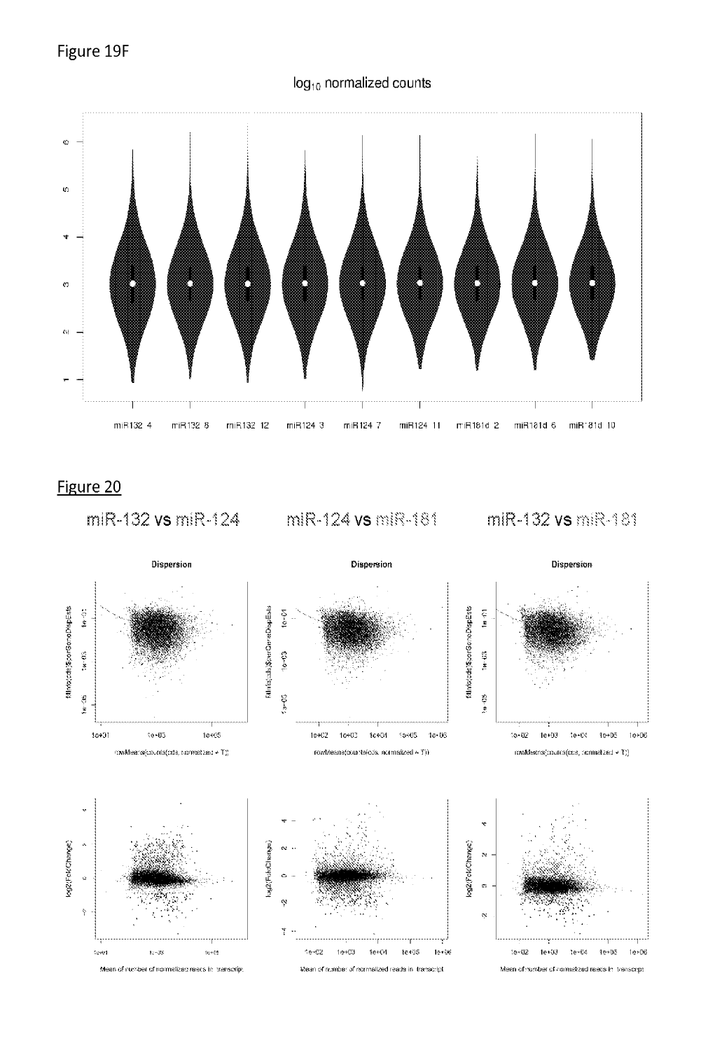

Approximately 40-50 million reads were obtained per sample; on average, 75% uniquely mapped to the RefSeq annotation (FIG. 18 and Table 1). A baseline for the datasets was empirically determined and non-polyadenylated transcripts were filtered out bioinformatically. The data were normalized broadly using DESeq (Anders S and Huber W, Genome Biol 11, R106 (2010), incorporated by reference herein.) The distribution was analyzed and the normalization evaluated using violin plots. In addition, principal components analyses were used to evaluate the retention of clustering among conditions (FIG. 19). Transcript specific variance estimates were obtained by fitting the negative binomial model implemented in DESeq (FIG. 20).

TABLE 1
Number of reads per RISCtrap sample.

| | | Se- | | | Uniquely | |
|---|---|---|---|---|---|---|
| Sam- | | quencing | Condi- | Total | mapped | % |
| ple | Date | Lane | tion | Reads | reads | mapped |
| 4 | Sep. 30, | 1 | miR-132 | 49238950 | 38751139 | 78.7 |
| | 2011 | | | | | |
| 8 | Sep. 16, | 2 | miR-132 | 47874130 | 36232555 | 75.7 |
| | 2011 | | | | | |
| 12 | Sep. 25, | 3 | miR-132 | 59463409 | 31767959 | 53.4 |
| | 2011 | | | | | |
| 3 | Sep. 30, | 1 | miR-124 | 28298431 | 22870389 | 80.8 |
| | 2011 | | | | | |
| 7 | Sep. 16, | 2 | miR-124 | 37706512 | 28860734 | 76.5 |
| | 2011 | | | | | |
| 11 | Sep. 25, | 3 | miR-124 | 30225288 | 22920129 | 75.8 |
| | 2011 | | | | | |
| 2 | Oct. 7, | 1 | miR-181d | 48720171 | 30990325 | 63.6 |
| | 2011 | | | | | |
| 6 | Sep. 16, | 2 | miR-181d | 36589447 | 28120433 | 76.9 |
| | 2011 | | | | | |
| 10 | Oct. 7, | 3 | miR-181d | 37560483 | 28389637 | 75.6 |
| | 2011 | | | | | |

Significantly enriched transcripts for each microRNA were determined with pairwise comparisons among the triplicates using ANOVA (FDR<0.15) and combined with an experimentally determined 2-fold enrichment cutoff (Table 2). This strict 2-fold enrichment cutoff was determined by investigating the validation rate of randomly selected target mRNAs representing a wide range of fold-enrichments from the screens. The analysis of miR-181 is shown as an example in Table 2. Targets that exhibited at least a 2-fold enrichment using RISCtrap validated at a greater than 90% by quantitative PCR. Target mRNAs that enriched 1.5-1.8 fold with one microRNA relative to the other two microRNAs level validated by qPCR at a 12.5% rate. Similarly, target mRNAs showing 2-fold enrichment were far more likely to contain a canonical MRE motif relative to target mRNAs showing less than two-fold enrichment. Therefore those target mRNAs that were more than two fold enriched relative to a control microRNA were considered “high confidence” target mRNAs of the particular microRNA of interest.

TABLE 2
Identification of microRNA recognition elements (MRE)
motifs enriched among RISCtrap targets for miR-181.

| | RISC-trap | | | # total | % transcripts | Mode # |
|---|---|---|---|---|---|---|
| | Fold | | | predicted | with predicted | MRE's |
| | enrichment | % validated | | MRE's | MRE's | per transcript |
| miR-181 | >5 | 100% | (16/16) | 207 | 100% (16/16) | 6 |
| | 2.2-2.5 | 93% | (14/15) | 22 | 73% (11/15) | 1 |
| | 1.5-1.8 | 12.5% | (2/16) | 13 | 44% (7/16) | 0 |

High confidence lists of target mRNAs for each of miRNA-124, miR-132, and miR-181 were finalized by requiring more than one pairwise comparison to indicate enrichment for the target mRNA. That is, to be listed as a target mRNA for miR-124, the target mRNA needed to be enriched relative to both miR-181 and miR-132. The gene symbols for the 281 high confidence target mRNAs identified by RISCtrap for miR-124 are as follows: RHOG, ERAL1, LCN15, CEBPA, GGA2, CTDSP1, SNAI2, SLC17A5, C17orf28, TSKU, B4GALT1, MAPK1IP1L, PLOD3, APEX2, ELOVL1, LGALS3, STX2, C11orf67, LIF, DTX2, BVES, APBA3, RNF135, PPP1R3B, FAM82A1, RRAS, RNPEPL1, C20orf29, TRIB3, LEPRA|LEPROT, TGDS, PIM2, MGC57346, CRHR1, SGPP2, SIX4, FAM189A2, SLC43A1, TMEM69, NFIC, OAF, FAM60A, PIP4K2C, PODXL, FBLIM1, LRRC1, RNH1, TARBP1, BRAT1, TSC22D4, TRPS1, PAPD4, ZNF784, SCAMP2, FSTL3, TRIM45, ZNF131, RBM47, MSRB3, FRMD8, ABHD4, NLRX1, KIF13B, MAML1, SLC16A10, SCN4B, ZCCHC24, HEATR6, RAB5C, CHST14, RAET1KI, TMEM134, LITAF, ZNF449, LINC00174, NR4A1, ZSCAN22, SLC2A4RG, FCHO2, YEATS2, GAS2L1, SYNGR2, PPP1R13L, FAM83H, ULBP1, MAVS, STX10, PTBP1|MIR4745, MKL2, ARHGEF37, SLC50A1, TMEM14B, CD164, RAD51AP1, TMEM109, TLN1, ATP8B2, CTNS, CRTC3, SP1, SLC25A16, TMEM161A, IDS, TFEB, PLEKHA7, SLC10A7, ZFP36L2, TMBIM1, PARM1, SALL4, PGF, LOC100506469, SLC16A13, ARAF, FLT3LG, LRRC42, GLIS2, QSER1, C2orf81, NEK6, CGN, TMED1, LAGE3, DVL2, PUS10, C11orf9, PLXNA3, CC2D2A, RHBDF1, RNF24, GK5, NEURL1B, AMOTL1, ARFIP1, MFGE8, NKTR, VAT1, LCLAT1, FAM35A, ATP6V0E1, SLC29A1, STK36, C4orf46, CERS2, SLC27A1, INPP5D, TSHZ2, AHR, DECR1, CD151, SGMS2, PLXNB2, CPEB1, RFX1, BCL11B, MYADM, WIPF1, WTIP, ABHD5, AXIN1, LSM14B, ZBTB7B, KDM6B, VPS37C, MAPK14, C17orf69, C3orf38, COL4A1, RNF139, KLF15, GSTK1, NFIA, C11orf70, PGAP1, STYX, SLC35A4, MGC72080, FAM35B2, FNDC3B, PAQR7, FUT4, NME4, CTSH, HADHB, FAM160A1, EML6, ATRIP, MORC4, PLAG1, TMEM179B, PPAP2B, CDCA7, C10orf26, SLC26A2, FAM171A1, ELK3, TBX19, TM7SF3, H6PD, ZNF280B, C5orf4, PABPC4L, K1AA0247, C2orf68, E2F5, TYK2, THAP2, MRI1, KIAA1530, KIAA1804, SGK1, SERP1, ZBED3, PDE3B, SLC25A30, BCL6B, LOC100129034, HIPK1, FAM195A, SLC16A6, RANBP10, CNKSR3, PTCD3, RAB11FIP5, SNTB2, CHIC1, MOCS1, DISP1, FGD6, TEAD1, DHX40, LRRC40, FAM104A, RETSAT, FES, CDCA7L, LOC255512, RASSF5, ZBTB16, AURKA, CCND2, DDOST, CREB3L2, FLJ23152, C6orf204, SFSWAP, F11R, PLEKHH1, ZNF833P, CCNG2, COPS7A, ATG4A, RHOC, MBD6, MBNL2, VAMP3, BICD2, PCOLCE, STK38, ARHGDIA, TPK1, C7orf49, SRGAP1, RAB27A, PSKH1, CYP4V2, THTPA, NBR2, PLEKHF2, ZNF438, ZSCAN30, FAM120A, SMARCAD1, REEP3, C19orf54, PARP3, EDA2R, BTG2, ACTA2, TP5313, CC2D1B, and GDF15.

The gene symbols for the 92 high confidence target mRNAs identified by RISCtrap for miR-132 are as follows: TP53RK, FIS1, PRR16, INPP5K, C1D, 1L12RB2, PNKD, DIABLO, SMN2, RG9MTD1, TJAP1, ATRNL1, SERP2, FERMT2, MTMR1, PSMA2, CRK, ESYT2, TMEM87A, ACAD9, SH3BP5, METTL22, TOMM70A, ZNF746, CERKL, EBPL, DIMT1, ARHGEF11, STMN1|MIR3917, FAIM, YAP1, C22orf39, MEPCE, PLAA, TMEM99, SERPINI1, PHTF2, RRS1, RASSF8, RIPK2, TMEM50B, LOC100127983, RNF13, ZNF568, HMGB3, PRDM15, COA5, NVL, ORC5, WDR47, SAP30L, HBEGF, NFE2L2, MMGT1, LSM14A, RNF125, RSRC2, BRI3, PTRH1, TMEM136, HSPD1, PSMD4, KITLG, COX7A2L, C6orf225, MEX3A, SAMD12, RASA1, C5orf13, NBN, DUT, CCNY, C16orf87, GHITM, HAUS2, POM121C, LOC283335, DDHD1, TMEM19, DERL1, USP37, LGR4, PNPLA3, EIF2S3, LDLR, CDKN1A, PRKX, GDF15, TP5313, ACTA2, BTG2, EDA2R, INPP5D, and ARFIP1.

The gene symbols for the 264 high confidence target mRNAs identified by RISCtrap for mir-181 are as follows: ZNF14, ZNF709, ZNF564, ZNF439, ZNF658, ZNF44, ZNF823, ZNF12, ZFP37, ZNF3, ZNF283, ZNF699, ZNF101, ZNF700, LOC389458|RBAK-LOC389458|RBAK, ZNF124, ZNF850, ZNF180, ZNF780B, ZNF443, ZNF607, ZNF383, ZIC2, ZNF546, ZNF433, ZNF833P, ZNF442, ZNF620, ZNF780A, ZNF157, ZNF440, ZNF625|ZNF625-ZNF20|ZNF20, ZNF583, ZNF799, ZNF77, ZNF778, ZNF619, ZNF623, ZNF829, LOC652276, ZNF121, ZNF420, ZNF487P, ZNF563, ZNF606, ZNF266, ZFP90, ZNF58, TMEM161B, ZNF470, RFT1, ZNF345, ZNF594, ZNF182, ZNF33B, ZNF511, ADH5, ZNF627, NARF, ZNF791, ZNF404, ZNF621, ZNF146, ZFP30, KLF15, ZNF527, ZNF490, ZNF204P, KANK1, ZNF30, ZNF571, ZNF441, KRTCAP2, ZNF184, ZNF83, ANKRD43, ZNF616, HIC2, ARSJ, FUT1, ZNF717, C5orf34, CSRNP2, ADRBK1, ZNF567, ZNF317, CDADC1, NCALD, ZNF565, ZIC5, ZNF33A, FH, ZNF630, KCNQ5, ZNF570, PPIE, ZNF569, ZNF260, TMEM18, ZFP28, C7orf73, ZFP2, CTGF, ZFAND3, ZNF670-ZNF695 IZNF695|ZNF670, ZNF177|ZNF559-ZNF177|ZNF559, RGMB, IL17RB, CNOT1, ZNF883, GMPR, ZNF136, ZNF37BP, DPAGT1, ZNF461, ZNF669, LOC728392, ENTPD6, CISH, IRS2, ZNF436, BEND3, ZNF426, ZNF181, ZNF16, ANXA11, POLR2E, MIR198|FSTL1, NR6A1, MED8, ZNF551, LTBP1, FST, PHLDA1, ZKSCAN1, ZNF382, FAM35B2, SLC16A6, CHCHD10, LOC441204, CDKL2, MEGF9, CROCCP2, AFTPH, OCEL1, ZNF555, ZNF140, MERTK, ZNF252, SLC25A32, ZNF41, EFEMP1, GGCT, TXNDC15, USP6, SLC35C2, KHDC1, ZNF432, GLI4, ABHD3, KIF9, NAA40, C8orf59, ZSCAN30, C1orf109, ZNF449, C1orf50, ZNF790, PQLC1, ARHGEF37, CCDC126, SIPA1L2, ZDHHC7, AHCY, ZNF562, ZNF10, FZD6, PKD2, ARV1, ZFP36L2, TSNAXIP1, FBX034, RPA3, ZNF23, ZNF561, ZNF200, ZNF830, TXNDC12, MAFB, ARF6, ZNF35, RASEF, TLCD1, RARS, ZNF624, C12orf35, RBL2, LOC100287846, SS18L2, PSAP, PRKAG2, OCLN, SOX18, CDKN3, MY01C, FBX033, DUSP5, ARIH2, PANK3, ZNF572, FAM171A1, ANKLE2, NUDT19, ZNF197, KLF3, RFX5, ZBTB1, RABEPK, SIN3B, ZNF684, KLF2, ZNF557, OSBPL3, GALNT3, ZNF615, ZNF514, ADAMTS19, ETFDH, LYSMD2, PEA15, FAM150A, CDX2, FEM1B, CARM1, ZNF554, OGFRL1, BRD1, GLRB, NIPBL, KCNK5, COR06, EGR1, FAM160A1, TPBG, C14orf43, MOB3B, NEURL1B, UHRF2, CPNE2, PPAP2B, LIPT2, ZNF480, ZNF510, ERO1LB, CYR61, NAAA, CBX7, TAF5, SRSF7, ZNF568, DERL1, and WDR47.

While a number of potential target mRNAs were enriched relative to only one of the two other microRNAs, such mRNA targets were validated by PCR at a rate of about 50%.

Among the high confidence miRNA targets, were many previously published and novel target mRNAs of the microRNAs of interest (FIG. 22). Analysis of the miR-124 target mRNAs listed above revealed substantial overlap between the cohort of target mRNAs identified using RISCtrap and previously published miR-124 datasets obtained using other methods in different cell types. The set of miR-124 target mRNAs identified using RISCtrap overlapped by 99 target mRNAs with a set identified using an Ago2-immunoprecipitation approach (Karginov et al, 2007 supra). The set of miR-124 target mRNAs obtained using RISCtrap overlapped by 53 target mRNAs with a set identified using HITS-CLIP on murine brain (BC=4) (Chi et al, 2009 supra). The set of miR-124 target mRNAs obtained using RISCtrap overlapped by 45 mRNAs with a set of target mRNAs identified using a microarray in HeLa cells (Lim et al, 2005 supra). Finally, the set of miR-124 target mRNAs overlapped by 64 targets with a set of target mRNAs identified using proteomics in HeLa cells (Baek et al, 2008, supra).

The set of target mRNAs of miR-181 identified using RISCtrap revealed that almost half of the target mRNAs (125/262) were C2H2 class zinc-finger protein with 95 of these C2H2 class zinc-finger proteins having an N-terminal KRAB domain followed by multiple tandem C2H2 motifs. Other studies using microarray and luciferase based assays to identify target mRNAs of miR-181 also reported identifying this cohort of target mRNAs (Baek et al, 2008, supra; Huang et al, 2010 supra; and Schnall-Levin et al, 2011 supra).

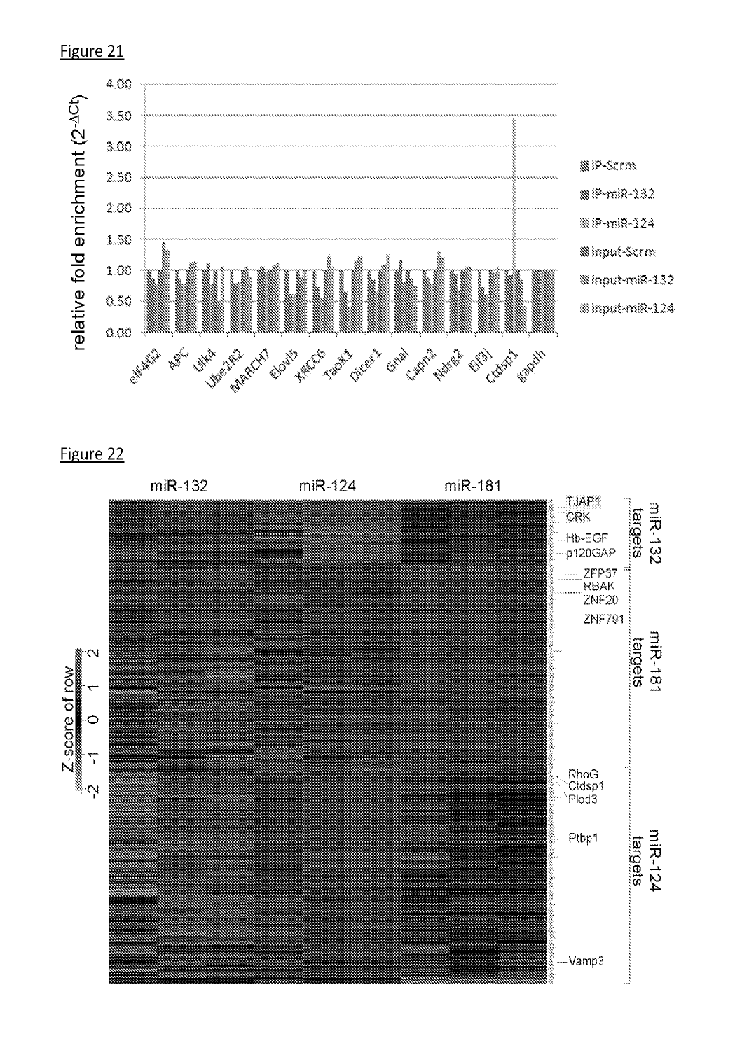

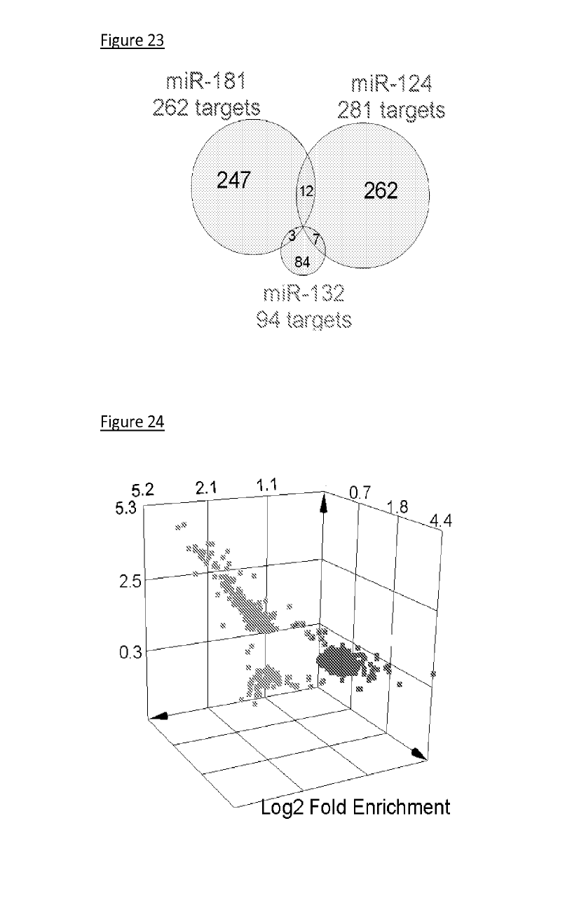

Examination of the sets of target mRNAs of miR-124, miR-132, and miR-181 revealed several interesting features. First, grouping of target mRNAs by biological replicates produced an enrichment profile for each transcript that was extremely reproducible and the cohorts of candidate targets clearly segregated based on the miRNA of interest (FIG. 22). In addition, the number of target mRNAs for each microRNA of interest differed between the three miRNAs, ranging from about 100 for miR-132 to almost 300 for miR-124 and miR-181. Further, there was minimal overlap among the target mRNA sets for each miRNA of interest (FIG. 23). Because miR-124 and miR-132 are active in the same neuronal cell types, one might predict that they many share many targets. However, only seven target mRNAs were found to be shared between the miR-124 and miR-132 datasets and there was no overlap among all three sets of miR-124, miR-132, and miR-181 targets. Finally, the range of enrichment values for each mRNA also varied depending on the identity of the microRNA (FIG. 24). For example, miR-181 target mRNAs exhibited a wide range of enrichments. In contrast, miR-132 targets averaged one MRE per gene and tended to cluster between a 2-8 fold enrichment.

A set of highly enriched, moderately enriched, and modestly enriched target mRNA were selected from the dataset identified for each microRNA screened. A second RISCtrap screen was run for each microRNA and the selected target mRNA validated using qPCR (FIGS. 25, 26, and 27). Overall, 149 target mRNA were selected from the first screen and 96% of those that displayed with 2 fold enrichment by RISCtrap also displayed 2 fold enrichment by qPCR. An additional three mRNA transcripts—gapdh, DHHC9, and DHHC17—that were not enriched in RISCtrap screens were included in the qPCR as negative controls; none of these transcripts showed any enrichment in either the RISCtrap screen or qPCR validation.

### Example 14

### Characterization of MicroRNA Regulatory Elements (MREs) Using RISCtrap

The sets of target mRNAs identified for miR-124, miR-132, and miR-181 were examined for whether or not they contained expected microRNA binding motifs. Canonical MRE's as well as described pivot (or hinged) MREs were examined (Chi S W et al, Nat Struct Mol Biol 18, 1218-1226 (2012); incorporated by reference herein). Approximately 90% of all targets contained an MRE that corresponded to the microRNA of interest: 91.5% of miR-124 targets had a canonical miR-124 MRE, 87.2% of the miR-132 targets had a canonical miR-132 MRE, and 92.4 of the miR-181 targets had a canonical miR-181 MRE (FIG. 28). The majority of target mRNAs contained at least an 8-mer, 7-mer-m8, or 7mer-al type motifs; fewer had only a 6-mer or pivot MRE (7% of miR-124 target mRNAs, 20% of miR-132 target mRNAs, and 2% of miR-181 target mRNAs). The frequency of 7mer-m8 motifs among non-targeted transcript pools was low, indicating that the appropriate MRE motifs were specifically enriched among targeted transcripts. Additionally, 82% of targets predicted to be co-regulated by at least two of miR-124, miR-132, and miR-181 contained MRE motifs for both microRNAs (Table 3).

TABLE 3
Target sequences predicted to be co-regulated by two distinct miRNAs were
examined for inclusion of MRE motifs corresponding to both miRNAs.

| | # shared | Both | miR-181 | miR-124 | miR-132 | |
|---|---|---|---|---|---|---|
| miRNA pair | targets | miRNA | only | only | only | No MREs |
| miR-124 + miR-181 | 12 | 11 | 1 | 0 | N/A | 0 |
| miR-132 + miR-181 | 3 | 3 | 0 | N/A | 0 | 0 |
| miR-132 + miR-124 | 7 | 4 | N/A | 1 | 1 | 1 |

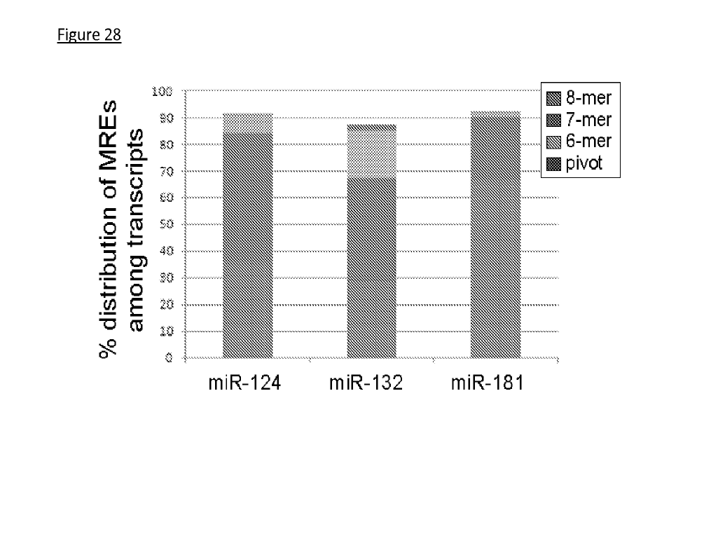

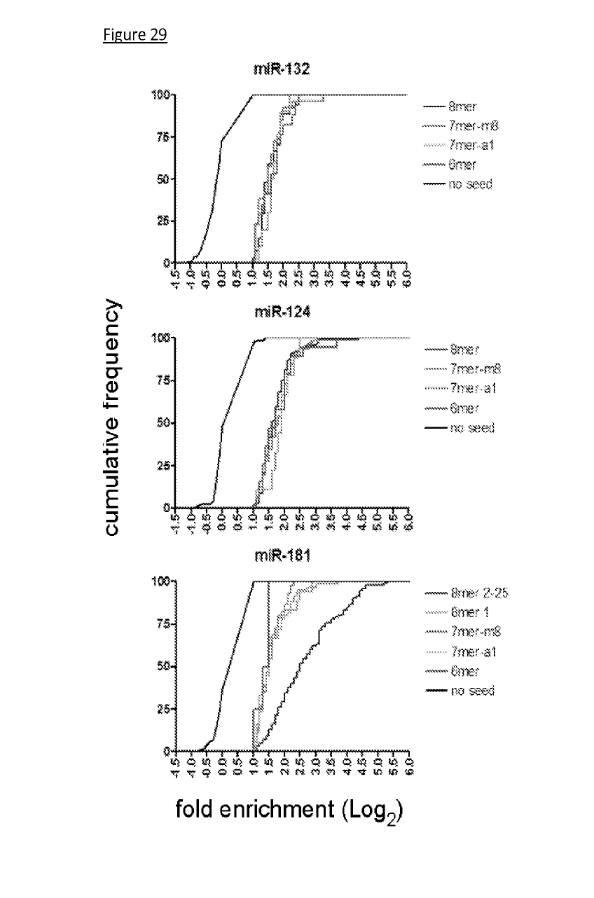

Plotting the cumulative frequency of motif types against the observed fold-enrichments revealed that all motifs were represented equally well in the RISCtrap purifications, indicating that the assay is sufficiently sensitive to enrich for 6-mer as well as 8-mer motifs (FIG. 29). In addition, it was found that many C2H2 zinc-finger miR-181 targets had multiple MRE motifs; 115 miR-181 targets had 2-25 predicted MREs per transcript. Among this unusual group of targets there was a strong correlation with the number of MRE motifs and fold-enrichment in RISCtrap. Investigation into the total number of MRE motifs per target further revealed surprising differences among the microRNA datasets. Targets of miR-124 and miR-132 averaged approximately 1 MRE per target; in contrast, miR-181 targets averaged 5.5 MREs per target (FIG. 31). In addition, pivot MREs were identified in 12-25% of target mRNAs (often along with a canonical MRE); the observed frequency of this motif was similar to published reports (Chi et al, 2012 supra) (FIG. 30).

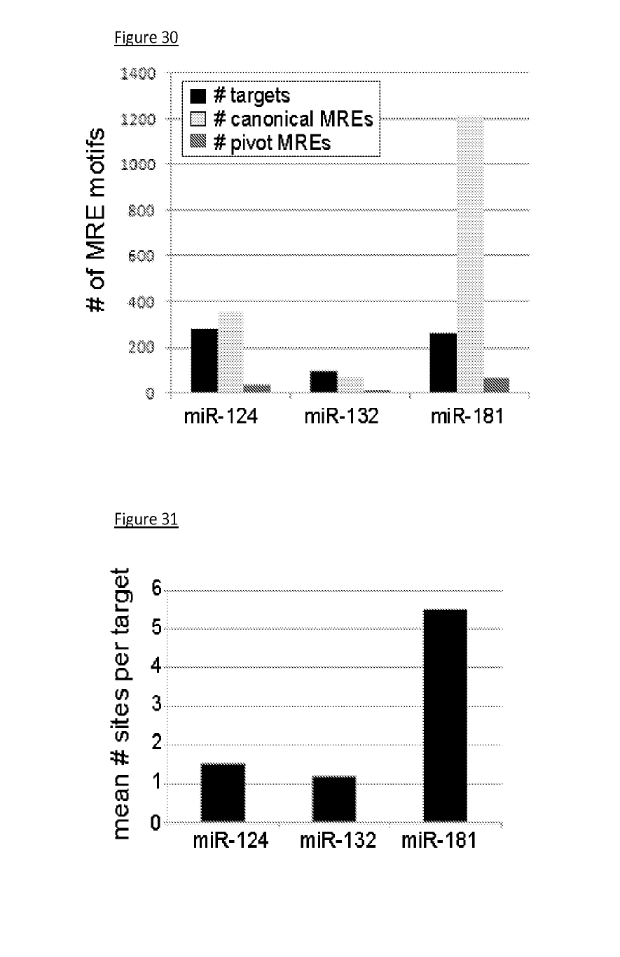

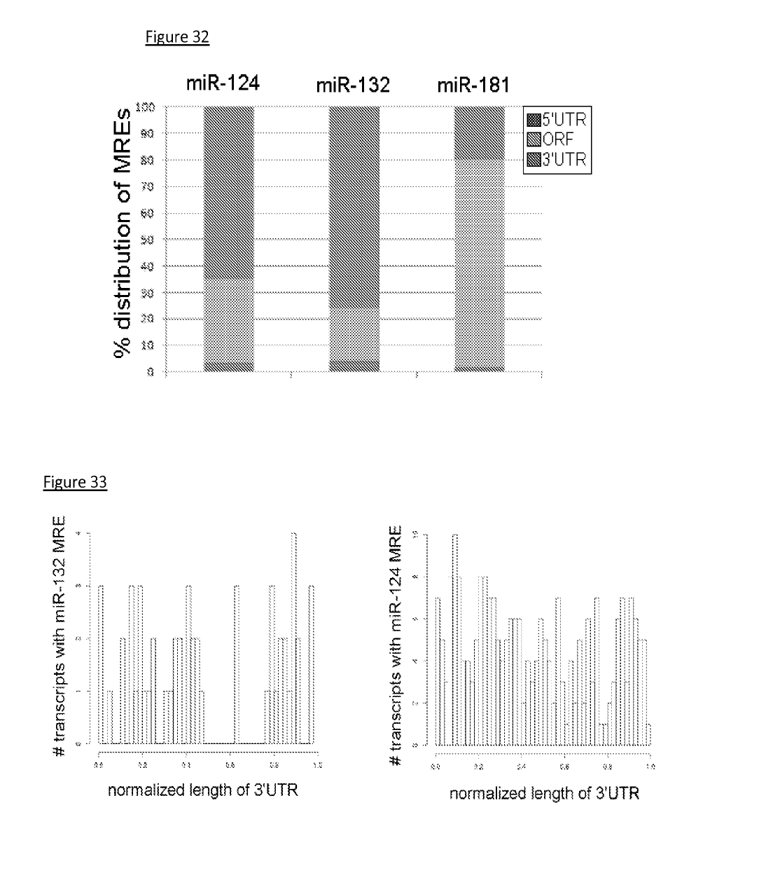

The distribution of 7mer-m8 sites among the miR-124 and miR-132 target mRNAs was also examined. The majority of MREs (60-80%) were located in the 3′UTR, with about 20-30% in open reading frames (ORFs) (FIG. 32). For these microRNAs, the relative position of MRE motifs along 3′UTRs appeared evenly dispersed (FIG. 33). Conversely, the majority of miR-181 targets contained MREs in the ORFs and, in agreement with previously published reports, were specifically encoded within the C2H2 motif repeats (FIG. 34) (Huang et al, 2010 supra and Schnall-Levin et al, 2011 supra).

De novo MEME analyses were used to identify overly represented sequences. This identified motifs that corresponded to canonical MREs for both miR-124 and miR-181 in the 3′UTRs of their respective targets, as well as many more in the ORFs of miR-181 targets. There was no miR-132 motif identified with the de novo analysis, despite its high representation when performing a directed search. Most likely, this is due to a relatively higher reliance on 6-mer motifs compared to miR-124 and miR-181 and this shorter motif is not easily distinguished by de novo analysis.

### Example 15

### New miR-132 Targets CRK and TJAP1 are Regulated by miR-132 In Vitro

As discussed above, RISCtrap screens of miR-124, miR-132 and miR-181 identified many previously known targets. In addition, RISCtrap screens identified novel target mRNAs of these microRNAs, many of which were enriched to a level exceeding that of known target mRNAs of these microRNAs. Two of the miR-132 target mRNAs identified in the RISCtrap screen—CRK and TJAP1 were selected for further investigation.

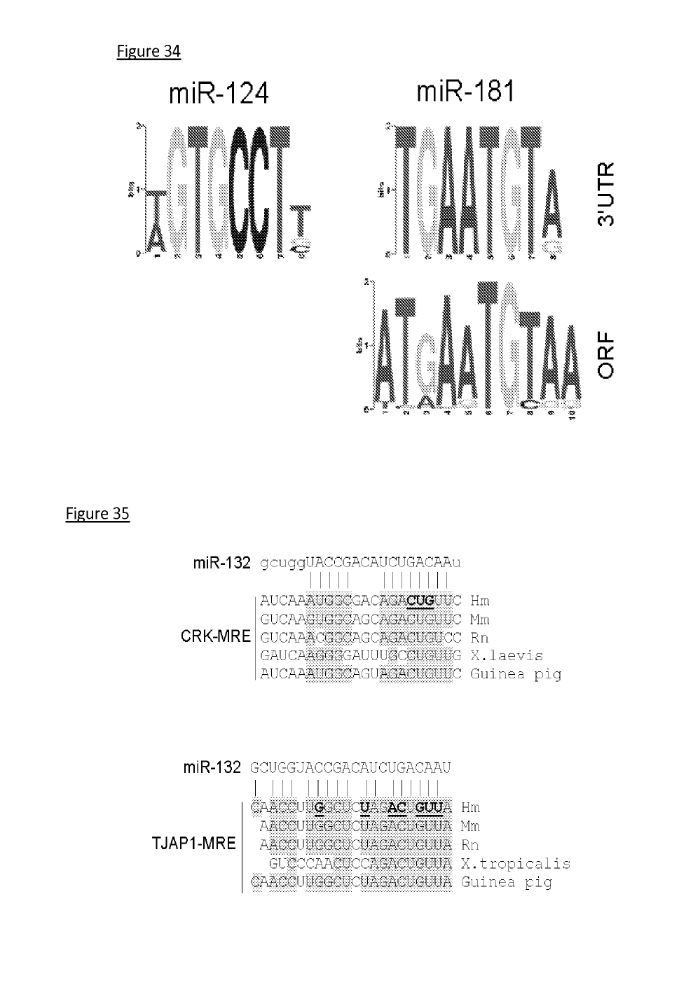

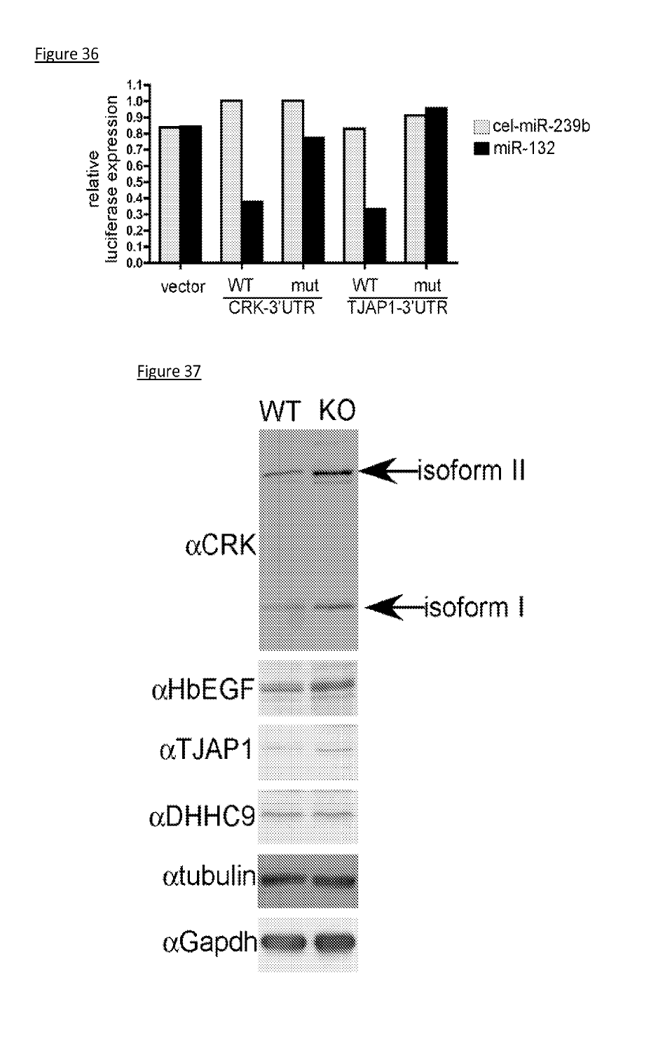

CRK is an adaptor protein for receptor tyrosine kinases and TJAP1 associates with tight junctions. Both candidates were validated by qPCR and available microarray data indicated that both mRNAs are expressed at high levels in brain (Su Al et al, Proc Natl Acad Sci USA 101, 6062-6067 (2004) and Wu C et al, Genome Biol 10 R130 (2009), both of which are incorporated by reference herein). Moreover, each of these two target mRNAs has a well conserved MRE site in its 3′UTR (FIG. 35). Incorporation of the 3′UTR sequence for either CRK or TJAP1 downstream of renilla luciferase in a dual luciferase assay confirmed miR-132 regulation (WT). Mutation of the MRE (mut) caused the MRE to be refractory to regulation by miR-132 (FIG. 36).

### Example 16

### New miR-132 Targets CRK and TJAP1 are Regulated by miR-132 In Vivo

Examination of endogenous protein from whole cell lysates of litter-matched male siblings revealed an accumulation of CRK and TJAP1 in a miR-132 knockout animal (FIG. 37).

### Example 17

### Control for Addition of Exogenous miRNA Leading to Spurious miRNA-Target mRNA Interactions

To test whether addition of exogenous miRNA caused spurious interactions, thirteen miR-124 target mRNAs identified in a previous study using HITS-CLIP (BC=5) (Chi et al, 2009 supra) that were known to be expressed in HEK293T cells but absent from the set of target mRNAs identified in the miR-124 RISCtrap screen described here, were assessed by qPCR (FIG. 21). None of these candidate targets demonstrated enrichment despite ectopic miR-124 expression, suggesting that all miRNA-target mRNA interactions were true silencing reactions. Although it has been suggested that methods that involve crosslinking of miRNA-target mRNAs have an advantage of not requiring addition of exogenous miRNAs, the addition of exogenous miRNA in the RISCtrap system did not cause spurious interactions.

1. A method of identifying a target mRNA of a microRNA of interest, the method comprising:
contacting the microRNA of interest with a dominant negative GW182 polypeptide within a cell;
purifying a complex comprising the dominant negative GW182 polypeptide and an endogenously expressed target mRNA of the microRNA of interest; and
identifying the endogenously expressed target mRNA.

2. The method of claim 1 wherein the dominant negative GW182 polypeptide shares at least 30% sequence homology with SEQ ID NO: 1.

3. The method of claim 2 wherein the dominant negative GW182 polypeptide shares at least 50% sequence homology with one or more of SEQ ID NO: 2, SEQ ID NO: 3, SEQ ID NO: 4, SEQ ID NO: 5, SEQ ID NO: 6 and SEQ ID NO: 7.

4. The method of claim 1 wherein the mutation in the silencing domain comprises a mutation in the RRM domain of the dominant negative GW182 polypeptide.

5. The method of claim 1 wherein the mutation in the silencing domain comprises an amino acid substation mutation.

6. The method of claim 1 wherein the mutation in the silencing domain comprises a deletion.

7. The method of claim 6 wherein the deletion is a deletion of less than 550 amino acids.

8. The method of claim 6 wherein the deletion is a deletion of less than 100 amino acids.

9. The method of claim 6 wherein the entire RRM domain is deleted.

10. The method of claim 6 wherein the entire silencing domain is deleted.

11. The method of claim 1 wherein the dominant negative GW182 polypeptide shares at least 30% sequence homology with SEQ ID NO: 8.

12. The method of claim 1 wherein the dominant negative GW182 polypeptide shares at least 50% sequence homology with a sequence selected from the group consisting of SEQ ID NO: 9, SEQ ID NO: 10, and SEQ ID NO: 11.

13. The method of claim 1 wherein contacting the microRNA of interest with the dominant negative GW182 polypeptide comprises transfecting the cell with a first nucleic acid construct, the first nucleic acid construct comprising a first polynucleotide sequence, the first polynucleotide sequence comprising the sequence of the microRNA of interest.

14. The method of claim 13 wherein the first polynucleotide sequence is a pre-microRNA sequence of the microRNA of interest.

15. The method of claim 13 wherein the first polynucleotide sequence is a mature sequence of the microRNA of interest.

16. The method of claim 13 wherein transfecting the cell with the first nucleic acid construct is performed through a transfection method selected from: transfection with a transfection agent, electroporation, or calcium phosphate transfection.

17. The method of claim 1 wherein contacting the microRNA of interest with the dominant negative GW182 polypeptide further comprises transfecting a cell with a second nucleic acid construct, the second nucleic acid construct comprising a second polynucleotide sequence that encodes the dominant negative GW182 polypeptide and third polynucleotide sequence that is a promoter operably linked to second polynucleotide sequence.

18. The method of claim 17 wherein the second nucleic acid construct is stably transfected.

19. The method of claim 17 wherein the sequence that encodes the dominant negative GW182 polypeptide has at least 30% homology to SEQ ID NO: 12.

20. The method of claim 17 wherein the sequence that encodes the dominant negative GW182 polypeptide has at least 50% sequence homology to a sequence selected from SEQ ID NO: 13, SEQ ID NO: 14, or SEQ ID NO: 15.

21. The method of claim 17 wherein the second nucleic acid construct further comprises a fourth polynucleotide sequence that is a sequence derived from a virus.

22. The method of claim 21 wherein the virus is selected from adenovirus and lentivirus.

23. The method of claim 21 wherein the virus is an adenovirus and wherein the promoter is a CMV promoter.

24. The method of claim 23 wherein the second nucleic acid construct comprises a sequence selected from SEQ ID NO: 16, SEQ ID NO: 17, and SEQ ID NO: 18.

25. The method of claim 22 wherein the virus is a lentivirus and wherein the second nucleic acid construct comprises a sequence selected from SEQ ID NO: 26, SEQ ID NO: 27, and SEQ ID NO: 28.

26. The method of claim 1 wherein purifying the complex comprises contacting the complex with a first reagent that specifically binds to a component of the complex.

27. The method of claim 26 wherein the first reagent comprises an antibody.

28. The method of claim 26 wherein the first reagent specifically binds to the dominant negative GW182 polypeptide.

29. The method of claim 26 wherein the dominant negative GW182 polypeptide comprises a label and wherein the first reagent specifically binds to the label.

30. The method of claim 29 wherein the label is selected from biotin, a myc tag, a FLAG® tag, or a His tag.

31. The method of claim 30 wherein the label is biotin and the dominant negative GW182 polypeptide is encoded by SEQ ID NO: 19, SEQ ID NO: 20, or SEQ ID NO: 21.

32. The method of claim 30 wherein the label is a myc tag and the dominant negative GW182 polypeptide is encoded by SEQ ID NO: 23, SEQ ID NO: 24, or SEQ ID NO: 25.

33. The method of claim 30 wherein the label is a His tag or FLAG® tag and wherein the dominant negative GW182 polypeptide is encoded by SEQ ID NO: 22.

34. The method of claim 1 wherein identifying the endogenously expressed target mRNA comprises a method selected from polymerase chain reaction, microarray analysis, or nucleic acid sequencing.

35. The method of claim 34 wherein identifying the endogenously expressed target mRNA comprises nucleic acid sequencing and wherein sequences that are enriched at least two-fold relative to a mean value of all sequences detected in the screen are identified as target mRNA.

36. The method of claim 1 wherein the microRNA of interest is a mutant form of microRNA relative to its native sequence.

37. The method of claim 1 further comprising confirming the regulation of the target mRNA by the microRNA of interest by transfecting the microRNA of interest into a cell and assessing the expression of a protein encoded by the target mRNA.

38. A kit that facilitates a method of identifying a target mRNA of a microRNA of interest, the kit comprising:
a first nucleic acid construct that encodes a dominant negative GW182 polypeptide,
a first reagent used to purify a complex comprising the dominant negative GW182 polypeptide and an endogenously expressed target mRNA of a microRNA of interest; and
a second reagent used to identify the endogenously expressed target mRNA.

39. The kit of claim 38 wherein the first reagent specifically binds to the first polypeptide.

40. The kit of claim 38 wherein the first reagent specifically binds to a component of the complex other than the dominant negative GW182 polypeptide.

41. The kit of claim 40 wherein the component of the complex other than the dominant negative GW182 polypeptide is selected from SEQ ID NO: 29 and SEQ ID NO: 30.

42. The kit of claim 38 wherein the dominant negative GW182 polypeptide comprises a label and wherein the first reagent specifically binds to the label.

43. The kit of claim 38 wherein the second reagent comprises an oligonucleotide.

44. The kit of claim 43 wherein the oligonucleotide is configured for a use selected from: a primer used in a polymerase chain reaction, an oligonucleotide used in nucleic acid sequencing, and an oligonucleotide conjugated to a solid substrate.

45. The kit of claim 44 wherein the second reagent is a primer used in nucleic acid sequencing comprising a sequence that facilitates next generation nucleic acid sequencing.

46. The kit of claim 38 further comprising a transfection reagent.

47. The kit of claim 38 further comprising a nucleic acid construct, the nucleic acid construct comprising the sequence of the microRNA of interest.

48. The kit of claim 38 further comprising instructions describing the performance of the method


SEQUENCE LISTING (447 sequences)

SEQ ID NO: 1 - protein, 1384 aa, Drosophila melanogaster
MREALFSQDGWGCQHVNQDTNWEVPSSPEPANKDAPGPPMWKPSINNGTDLWESNLRNGGQPAAQQVPKPSWGHTPSSNLGGTWGEDDDGADSSSVWTGGAVSNAGSGAAVGVNQAGVNVGPGGVVSSGGPQWGQGVVGVGLGSTGGNGSSNITGSSGVATGSSGNSSNAGNGWGDPREIRPLGVGGSMDIRNVEHRGGNGSGATSSDPRDIRMIDPRDPIRGDPRGISGRLNGTSEMWGHHPQMSHNQLQGINKMVGQSVATASTSVGTSGSGIGPGGPGPSTVSGNIPTQWGPAQPVSVGVSGPKDMSKQISGWEEPSPPPQRRSIPNYDDGTSLWGQQTRVPAASGHWKDMTDSIGRSSHLMRGQSQTGGIGIAGVGNSNVPVGANPSNPISSVVGPQARIPSVGGVQHKPDGGAMWVHSGNVGGRNNVAAVTTWGDDTHSVNVGAPSSGSVSSNNWVDDKSNSTLAQNSWSDPAPVGVSWGNKQSKPPSNSASSGWSTAAGVVDGVDLGSEWNTHGGIIGKSQQQQKLAGLNVGMVNVINAEIIKQSKQYRILVENGFKKEDVERALVIANMNIEEAADMLRANSSLSMDGWRRHDESLGSYADHNSSTSSGGFAGRYPVNSGQPSMSFPHNNLMNNMGGTAVTGGNNNTNMTALQVQKYLNQGQHGVAVGPQAVGNSSAVSVGFGQNTSNAAVAGAASVNIAANTNNQPSGQQIRMLGQQIQLAIHSGFISSQILTQPLTQTTLNLLNQLLSNIKHLQAAQQSLTRGGNVNPMAVNVAISKYKQQIQNLQNQINAQQAVYVKQQNMQPTSQQQQPQQQQLPSVHLSNSGNDYLRGHDAINNLQSNFSELNINKPSGYQGASNQQSRLNQWKLPVLDKEINSDSTEFSRAPGATKQNLTANTSNINSLGLQNDSTWSTGRSIGDGWPDPSSDNENKDWSVAQPTSAATAYTDLVQEFEPGKPWKGSQIKSIEDDPSITPGSVARSPLSINSTPKDADIFANTGKNSPTDLPPLSLSSSTWSFNPNQNYPSHSWSDNSQQCTATSELWTSPLNKSSSRGPPPGLTANSNKSANSNASTPTTITGGANGWLQPRSGGVQTTNTNWTGGNTTWGSSWLLLKNLTAQIDGPTLRTLCMQHGPLVSFHPYLNQGIALCKYTTREEANKAQMALNNCVLANTTIFAESPSENEVQSIMQHLPQTPSSTSSSGTSGGNVGGVGTSANNANSGSAACLSGNNSGNGNGSASGAGSGNNGNSSCNNSAAGGGSSSNNTITTVANSNLVGSSGSVSNSSGVTANSSTVSVVSCTASGNSINGAGTANSSGSKSSANNLASGQSSASNLTNSTNSTWRQTSQNQALQSQSRPSGREADFDYISLVYSIVDD

SEQ ID NO: 2 - protein, 1962 aa, Homo sapiens
MRELEAKATKDVERNLSRDLVQEEEQLMEEKKKKKDDKKKKEAAQKKATEQKIKVPEQIKPSVSQPQPANSNNGTSTATSTNNNAKRATANNQQPQQQQQQQQPQQQQPQQQPQPQPQQQQPQQQPQALPRYPREVPPRFRHQEHKQLLKRGQHFPVIAANLGSAVKVLNSQSESSALTNQQPQNNGEVQNSKNQSDINHSTSGSHYENSQRGPVSSTSDSSTNCKNAVVSDLSEKEAWPSAPGSDPELASECMDADSASSSESERNITIMASGNTGGEKDGLRNSTGLGSQNKFVVGSSSNNVGHGSSTGPWGFSHGAIISTCQVSVDAPESKSESSNNRMNAWGTVSSSSNGGLNPSTLNSASNHGAWPVLENNGLALKGPVGSGSSGINIQCSTIGQMPNNQSINSKVSGGSTHGTWGSLQETCESEVSGTQKVSFSGQPQNITTEMTGPNNTTNFMTSSLPNSGSVQNNELPSSNTGAWRVSTMNHPQMQAPSGMNGTSLSHLSNGESKSGGSYGTTWGAYGSNYSGDKCSGPNGQANGDTVNATLMQPGVNGPMGTNFQVNTNKGGGVWESGAANSQSTSWGSGNGANSGGSRRGWGTPAQNTGTNLPSVEWNKLPSNQHSNDSANGNGKTFTNGWKSTEEEDQGSATSQTNEQSSVWAKTGGTVESDGSTESTGRLEEKGTGESQSRDRRKIDQHTLLQSIVNRTDLDPRVLSNSGWGQTPIKQNTAWDTETSPRGERKTDNGTEAWGSSATQTFNSGACIDKTSPNGNDTSSVSGWGDPKPALRWGDSKGSNCQGGWEDDSAATGMVKSNQWGNCKEEKAAWNDSQKNKQGWGDGQKSSQGWSVSASDNWGETSRNNHWGEANKKSSSGGSDSDRSVSGWNELGKTSSFTWGNNINPNNSSGWDESSKPTPSQGWGDPPKSNQSLGWGDSSKPVSSPDWNKQQDIVGSWGIPPATGKPPGTGWLGGPIPAPAKEEEPTGWEEPSPESIRRKMEIDDGTSAWGDPSKYNYKNVNMWNKNVPNGNSRSDQQAQVHQLLTPASAISNKEASSGSGWGEPWGEPSTPATTVDNGTSAWGKPIDSGPSWGEPIAAASSTSTWGSSSVGPQALSKSGPKSMQDGWCGDDMPLPGNRPTGWEEEEDVEIGMWNSNSSQELNSSLNWPPYTKKMSSKGLSGKKRRRERGMMKGGNKQEEAWINPFVKQFSNISFSRDSPEENVQSNKMDLSGGMLQDKRMEIDKHSLNIGDYNRTVGKGPGSRPQISKESSMERNPYFDKDGIVADESQNMQFMSSQSMKLPPSNSALPNQALGSIAGLGMQNLNSVRQNGNPSMFGVGNTAAQPRGMQQPPAQPLSSSQPNLRAQVPPPLLSPQVPVSLLKYAPNNGGLNPLFGPQQVAMLNQLSQLNQLSQISQLQRLLAQQQRAQSQRSVPSGNRPQQDQQGRPLSVQQQMMQQSRQLDPNLLVKQQTPPSQQQPLHQPAMKSFLDNVMPHTTPELQKGPSPINAFSNFPIGLNSNLNVNMDMNSIKEPQSRLRKWTTVDSISVNTSLDQNSSKHGAISSGFRLEESPFVPYDFMNSSTSPASPPGSIGDGWPRAKSPNGSSSVNWPPEFRPGEPWKGYPNIDPETDPYVTPGSVINNLSINTVREVDHLRDRNSGSSSSLNTTLPSTSAWSSIRASNYNVPLSSTAQSTSARNSDSKLTWSPGSVTNTSLAHELWKVPLPPKNITAPSRPPPGLTGQKPPLSTWDNSPLRIGGGWGNSDARYTPGSSWGESSSGRITNWLVLKNLTPQIDGSTLRTLCMQHGPLITFHLNLPHGNALVRYSSKEEVVKAQKSLHMCVLGNTTILAEFASEEEISRFFAQSQSLTPSPGWQSLGSSQSRLGSLDCSHSFSSRTDLNHWNGAGLSGTNCGDLHGTSLWGTPHYSTSLWGPPSSSDPRGISSPSPINAFLSVDHLGGGGESM

SEQ ID NO: 3 - protein, 1833 aa, Homo sapiens
MREKEQEREEQLMEDKKRKKEDKKKKEATQKVTEQKTKVPEVTKPSLSQPTAASPIGSSPSPPVNGGNNAKRVAVPNGQPPSAARYMPREVPPRFRCQQDHKVLLKRGQPPPPSCMLLGGGAGPPPCTAPGANPNNAQVTGALLQSESGTAPDSTLGGAAASNYANSTWGSGASSNNGTSPNPIHIWDKVIVDGSDMEEWPCIASKDTESSSENTTDNNSASNPGSEKSTLPGSTTSNKGKGSQCQSASSGNECNLGVWKSDPKAKSVQSSNSTTENNNGLGNWRNVSGQDRIGPGSGFSNFNPNSNPSAWPALVQEGTSRKGALETDNSNSSAQVSTVGQTSREQQSKMENAGVNFVVSGREQAQIHNTDGPKNGNTNSLNLSSPNPMENKGMPFGMGLGNTSRSTDAPSQSTGDRKTGSVGSWGAARGPSGTDTVSGQSNSGNNGNNGKEREDSWKGASVQKSTGSKNDSWDNNNRSTGGSWNFGPQDSNDNKWGEGNKMTSGVSQGEWKQPTGSDELKIGEWSGPNQPNSSTGAWDNQKGHPLPENQGNAQAPCWGRSSSSTGSEVGGQSTGSNHKAGSSDSHNSGRRSYRPTHPDCQAVLQTLLSRTDLDPRVLSNTGWGQTQIKQDTVWDIEEVPRPEGKSDKGTEGWESAATQTKNSGGWGDAPSQSNQMKSGWGELSASTEWKDPKNTGGWNDYKNNNSSNWGGGRPDEKTPSSWNENPSKDQGWGGGRQPNQGWSSGKNGWGEEVDQTKNSNWESSASKPVSGWGEGGQNEIGTWGNGGNASLASKGGWEDCKRSPAWNETGRQPNSWNKQHQQQQPPQQPPPPQPEASGSWGGPPPPPPGNVRPSNSSWSSGPQPATPKDEEPSGWEEPSPQSISRKMDIDDGTSAWGDPNSYNYKNVNLWDKNSQGGPAPREPNLPTPMTSKSASVWSKSTPPAPDNGTSAWGEPNESSPGWGEMDDTGASTTGWGNTPANAPNAMKPNSKSMQDGWGESDGPVTGARHPSWEEEEDGGVWNTTGSQGSASSHNSASWGQGGKKQMKCSLKGGNNDSWMNPLAKQFSNMGLLSQTEDNPSSKMDLSVGSLSDKKFDVDKRAMNLGDFNDIMRKDRSGFRPPNSKDMGTTDSGPYFEKLTLPFSNQDGCLGDEAPCSPFSPSPSYKLSPSGSTLPNVSLGAIGTGLNPQNFAARQGGSHGLFGNSTAQSRGLHTPVQPLNSSPSLRAQVPPQFISPQVSASMLKQFPNSGLSPGLFNVGPQLSPQQIAMLSQLPQIPQFQLACQLLLQQQQQQQLLQNQRKISQAVRQQQEQQLARMVSALQQQQQQQQRQPGMKHSPSHPVGPKPHLDNMVPNALNVGLPDLQTKGPIPGYGSGFSSGGMDYGMVGGKEAGTESRFKQWTSMMEGLPSVATQEANMHKNGAIVAPGKTRGGSPYNQFDIIPGDTLGGHTGPAGDSWLPAKSPPTNKIGSKSSNASWPPEFQPGVPWKGIQNIDPESDPYVTPGSVLGGTATSPIVDTDHQLLRDNTTGSNSSLNTSLPSPGAWPYSASDNSFTNVHSTSAKFPDYKSTWSPDPIGHNPTHLSNKMWKNHISSRNTTPLPRPPPGLTNPKPSSPWSSTAPRSVRGWGTQDSRLASASTWSDGGSVRPSYWLVLHNLTPQIDGSTLRTICMQHGPLLTFHLNLTQGTALIRYSTKQEAAKAQTALHMCVLGNTTILAEFATDDEVSRFLAQAQPPTPAATPSAPAAGWQSLETGQNQSDPVGPALNLFGGSTGLGQWSSSAGGSSGADLAGASLWGPPNYSSSLWGVPTVEDPHRMGSPAPLLPGDLLGGGSDSI

SEQ ID NO: 4 - protein, 1723 aa, Homo sapiens
MREKEQEREEQLMEDKKRKKEDKKKKEATQKVTEQKTKVPEVTKPSLSQPTAASPIGSSPSPPVNGGNNAKRVAVPNGQPPSAARYMPREVPPRFRCQQDHKVLLKRGQPPPPSCMLLGGGAGPPPCTAPGANPNNAQVTGALLQSESGTAPDSTLGGAAASNYANSTWGSGASSNNGTSPNPIHIWDKVIVDGSDMEEWPCIASKDTESSSENTTDNNSASNPGSEKSTLPGSTTSNKGKGSQCQSASSGNECNLGVWKSDPKAKSVQSSNSTTENNNGLGNWRNVSGQDRIGPGSGFSNFNPNSNPSAWPALVQEGTSRKGALETDNSNSSAQVSTVGQTSREQQSKMENAGVNFVVSGREQAQIHNTDGPKNGNTNSLNLSSPNPMENKGMPFGMGLGNTSRSTDAPSQSTGDRKTGSVGSWGAARGPSGTDTVSGQSNSGNNGNNGKEREDSWKGASVQKSTGSKNDSWDNNNRSTGGSWNFGPQDSNDNKWGEGNKMTSGVSQGEWKQPTGSDELKIGEWSGPNQPNSSTGAWDNQKGHPLPENQGNAQAPCWGRSSSSTGSEVGGQSTGSNHKAGSSDSHNSGRRSYRPTHPDCQAVLQTLLSRTDLDPRVLSNTGWGQTQIKQDTVWDIEEVPRPEGKSDKGTEGWESAATQTKNSGGWGDAPSQSNQMKSGWGELSASTEWKDPKNTGGWNDYKNNNSSNWGGGRPDEKTPSSWNENPSKDQGWGGGRQPNQGWSSGKNGWGEEVDQTKNSNWESSASKPVSGWGEGGQNEIGTWGNGGNASLASKGGWEDCKRSPAWNETGRQPNSWNKQHQQQQPPQQPPPPQPEASGSWGGPPPPPPGNVRPSNSSWSSGPQPATPKDEEPSGWEEPSPQSISRKMDIDDGTSAWGDPNSYNYKNVNLWDKNSQGGPAPREPNLPTPMTSKSASDSKSMQDGWGESDGPVTGARHPSWEEEEDGGVWNTTGSQGSASSHNSASWGQGGKKQMKCSLKGGNNDSWMNPLAKQFSNMGLLSQTEDNPSSKMDLSVGSLSDKKFDVDKRAMNLGDFNDIMRKDRSGFRPPNSKDMGTTDSGPYFEKGGSHGLFGNSTAQSRGLHTPVQPLNSSPSLRAQVPPQFISPQVSASMLKQFPNSGLSPGLFNVGPQLSPQQIAMLSQLPQIPQFQLACQLLLQQQQQQQLLQNQRKISQAVRQQQEQQLARMVSALQQQQQQQQRQPGMKHSPSHPVGPKPHLDNMVPNALNVGLPDLQTKGPIPGYGSGFSSGGMDYGMVGGKEAGTESRFKQWTSMMEGLPSVATQEANMHKNGAIVAPGKTRGGSPYNQFDIIPGDTLGGHTGPAGDSWLPAKSPPTNKIGSKSSNASWPPEFQPGVPWKGIQNIDPESDPYVTPGSVLGGTATSPIVDTDHQLLRDNTTGSNSSLNTSLPSPGAWPYSASDNSFTNVHSTSAKFPDYKSTWSPDPIGHNPTHLSNKMWKNHISSRNTTPLPRPPPGLTNPKPSSPWSSTAPRSVRGWGTQDSRLASASTWSDGGSVRPSYWLVLHNLTPQIDGSTLRTICMQHGPLLTFHLNLTQGTALIRYSTKQEAAKAQTALHMCVLGNTTILAEFATDDEVSRFLAQAQPPTPAATPSAPAAGWQSLETGQNQSDPVGPALNLFGGSTGLGQWSSSAGGSSGADLAGASLWGPPNYSSSLWGVPTVEDPHRMGSPAPLLPGDLLGGGSDSI

SEQ ID NO: 5 - protein, 1029 aa, Homo sapiens
MQTNEGEVSEESSSKVEQEDFVMEGHGKTPPPGEESKQEKEQEREEQLMEDKKRKKEDKKKKEATQKVTEQKTKVPEVTKPSLSQPTAASPIGSSPSPPVNGGNNAKRVAVPNGQPPSAARYMPREVPPRFRCQQDHKVLLKRGQPPPPSCMLLGGGAGPPPCTAPGANPNNAQVTGALLQSESGTAPVWSKSTPPAPDNGTSAWGEPNESSPGWGEMDDTGASTTGWGNTPANAPNAMKPNSKSMQDGWGESDGPVTGARHPSWEEEEDGGVWNTTGSQGSASSHNSASWGQGGKKQMKCSLKGGNNDSWMNPLAKQFSNMGLLSQTEDNPSSKMDLSVGSLSDKKFDVDKRAMNLGDFNDIMRKDRSGFRPPNSKDMGTTDSGPYFEKGGSHGLFGNSTAQSRGLHTPVQPLNSSPSLRAQVPPQFISPQVSASMLKQFPNSGLSPGLFNVGPQLSPQQIAMLSQLPQIPQFQLACQLLLQQQQQQQLLQNQRKISQAVRQQQEQQLARMVSALQQQQQQQQRQPGMKHSPSHPVGPKPHLDNMVPNALNVGLPDLQTKGPIPGYGSGFSSGGMDYGMVGGKEAGTESRFKQWTSMMEGLPSVATQEANMHKNGAIVAPGKTRGGSPYNQFDIIPGDTLGGHTGPAGDSWLPAKSPPTNKIGSKSSNASWPPEFQPGVPWKGIQNIDPESDPYVTPGSVLGGTATSPIVDTDHQLLRDNTTGSNSSLNTSLPSPGAWPYSASDNSFTNVHSTSAKFPDYKSTWSPDPIGHNPTHLSNKMWKNHISSRNTTPLPRPPPGLTNPKPSSPWSSTAPRSVRGWGTQDSRLASASTWSDGGSVRPSYWLVLHNLTPQIDGSTLRTICMQHGPLLTFHLNLTQGTALIRYSTKQEAAKAQTALHMCVLGNTTILAEFATDDEVSRFLAQAQPPTPAATPSAPAAGWQSLETGQNQSDPVGPALNLFGGSTGLGQWSSSAGGSSGADLAGASLWGPPNYSSSLWGVPTVEDPHRMGSPAPLLPGDLLGGGSDSI

SEQ ID NO: 6 - protein, 1726 aa, Homo sapiens
MATGSAQGNFTGHTKKTNGNNGTNGALVQSPSNQSALGAGGANSNGSAARVWGVATGSSSGLAHCSVSGGDGKMDTMIGDGRSQNCWGASNSNAGINLNLNPNANPAAWPVLGHEGTVATGNPSSICSPVSAIGQNMGNQNGNPTGTLGAWGNLLPQESTEPQTSTSQNVSFSAQPQNLNTDGPNNTNPMNSSPNPINAMQTNGLPNWGMAVGMGAIIPPHLQGLPGANGSSVSQVSGGSAEGISNSVWGLSPGNPATGNSNSGFSQGNGDTVNSALSAKQNGSSSAVQKEGSGGNAWDSGPPAGPGILAWGRGSGNNGVGNIHSGAWGHPSRSTSNGVNGEWGKPPNQHSNSDINGKGSTGWESPSVTSQNPTVQPGGEHMNSWAKAASSGTTASEGSSDGSGNHNEGSTGREGTGEGRRRDKGIIDQGHIQLPRNDLDPRVLSNTGWGQTPVKQNTAWEFEESPRSERKNDNGTEAWGCAATQASNSGGKNDGSIMNSTNTSSVSGWVNAPPAAVPANTGWGDSNNKAPSGPGVWGDSISSTAVSTAAAAKSGHAWSGAANQEDKSPTWGEPPKPKSQHWGDGQRSNPAWSAGGGDWADSSSVLGHLGDGKKNGSGWDADSNRSGSGWNDTTRSGNSGWGNSTNTKANPGTNWGETLKPGPQQNWASKPQDNNVSNWGGAASVKQTGTGWIGGPVPVKQKDSSEATGWEEPSPPSIRRKMEIDDGTSAWGDPSNYNNKTVNMWDRNNPVIQSSTTTNTTTTTTTTTSNTTHRVETPPPHQAGTQLNRSPLLGPVSSGWGEMPNVHSKTENSWGEPSSPSTLVDNGTAAWGKPPSSGSGWGDHPAEPPVAFGRAGAPVAASALCKPASKSMQEGWGSGGDEMNLSTSQWEDEEGDVWNNAASQESTSSCSSWGNAPKKGLQKGMKTSGKQDEAWIMSRLIKQLTDMGFPREPAEEALKSNNMNLDQAMSALLEKKVDVDKRGLGVTDHNGMAAKPLGCRPPISKESSVDRPTFLDKDGGLVEEPTPSPFLPSPSLKLPLSHSALPSQALGGIASGLGMQNLNSSRQIPSGNLGMFGNSGAAQARTMQQPPQPPVQPLNSSQPSLRAQVPQFLSPQVQAQLLQFAAKNIGLNPALLTSPINPQHMTMLNQLYQLQLAYQRLQIQQQMLQAQRNVSGSMRQQEQQVARTITNLQQQIQQHQRQLAQALLVKQPPPPPPPPHLSLHPSAGKSAMDSFPSHPQTPGLPDLQTKEQQSSPNTFAPYPLAGLNPNMNVNSMDMTGGLSVKDPSQSQSRLPQWTHPNSMDNLPSAASPLEQNPSKHGAIPGGLSIGPPGKSSIDDSYGRYDLIQNSESPASPPVAVPHSWSRAKSDSDKISNGSSINWPPEFHPGVPWKGLQNIDPENDPDVTPGSVPTGPTINTTIQDVNRYLLKSGGSSPPSSQNATLPSSSAWPLSASGYSSSFSSIASAPSVAGKLSDIKSTWSSGPTSHTQASLSHELWKVPRNSTAPTRPPPGLTNPKPSSTWGASPLGWTSSYSSGSAWSTDTSGRTSSWLVLRNLTPQIDGSTLRTLCLQHGPLITFHLNLTQGNAVVRYSSKEEAAKAQKSLHMCVLGNTTILAEFAGEEEVNRFLAQGQALPPTSSWQSSSASSQPRLSAAGSSHGLVRSDAGHWNAPCLGGKGSSELLWGGVPQYSSSLWGPPSADDSRVIGSPTPLTTLLPGDLLSGESL

SEQ ID NO: 7 - protein, 1690 aa, Homo sapiens
MATGSAQGNFTGHTKKTNGNNGTNGALVQSPSNQSALGAGGANSNGSAARVWGVATGSSSGLAHCSVSGGDGKMDTMIGDGRSQNCWGASNSNAGINLNLNPNANPAAWPVLGHEGTVATGNPSSICSPVSAIGQNMGNQNGNPTGTLGAWGNLLPQESTEPQTSTSQNVSFSAQPQNLNTDGPNNTNPMNSSPNPINAMQTNGLPNWGMAVGMGAIIPPHLQGLPGANGSSVSQVSGGSAEGISNSVWGLSPGNPATGNSNSGFSQGNGDTVNSALSAKQNGSSSAVQKEGSGGNAWDSGPPAGPGILAWGRGSGNNGVGNIHSGAWGHPSRSTSNGVNGEWGKPPNQHSNSDINGKGSTGWESPSVTSQNPTVQPGGEHMNSWAKAASSGTTASEGSSDGSGNHNEGSTGREGTGEGRRRDKGIIDQGHIQLPRNDLDPRVLSNTGWGQTPVKQNTAWEFEESPRSERKNDNGTEAWGCAATQASNSGGKNDGSIMNSTNTSSVSGWVNAPPAAVPANTGWGDSNNKAPSGPGVWGDSISSTAVSTAAAAKSGHAWSGAANQEDKSPTWGEPPKPKSQHWGDGQRSNPAWSAGGGDWADSSSVLGHLGDGKKNGSGWDADSNRSGSGWNDTTRSGNSGWGNSTNTKANPGTNWGETLKPGPQQNWASKPQDNNVSNWGGAASVKQTGTGWIGGPVPVKQKDSSEATGWEEPSPPSIRRKMEIDDGTSAWGDPSNYNNKTVNMWDRNNPVIQSSTTTNTTTTTTTTTSNTTHRVETPPPHQAGTQLNRSPLLGPGRKVSSGWGEMPNVHSKTENSWGEPSSPSTLVDNGTAAWGKPPSSGSGWGDHPAEPPVAFGRAGAPVAASALCKPASKSMQEGWGSGGDEMNLSTSQWEDEEGDVWNNAASQESTSSCSSWGNAPKKGLQKGMKTSGKQDEAWIMSRLIKQLTDMGFPREPAEEALKSNNMNLDQAMSALLEKKVDVDKRGLGVTDHNGMAAKPLGCRPPISKESSVDRPTFLDKDGGLVEEPTPSPFLPSPSLKLPLSHSALPSQALGGIASGLGMQNLNSSRQIPSGNLGMFGNSGAAQARTMQQPPQPPVQPLNSSQPSLRAQVPQFLSPQVQAQLLQFAAKNIGLNPALLTSPINPQHMTMLNQLYQLQLAYQRLQIQQQMLQAQRNVSGSMRQQEQQVARTITNLQQQIQQHQRQLAQALLVKQPPPPPPPPHLSLHPSAGKSAMDSFPSHPQTPGLPDLQTKEQQSSPNTFAPYPLAGLNPNMNVNSMDMTGGLSVKDPSQSQSRLPQWTHPNSMDNLPSAASPLEQNPSKHGAIPGGLSIGPPGKSSIDDSYGRYDLIQNSESPASPPVAVPHSWSRAKSDSDKISNGSSINWPPEFHPGVPWKGLQNIDPENDPDVTPGSVPTGPTINTTIQDVNRYLLKSGGKLSDIKSTWSSGPTSHTQASLSHELWKVPRNSTAPTRPPPGLTNPKPSSTWGASPLGWTSSYSSGSAWSTDTSGRTSSWLVLRNLTPQIDGSTLRTLCLQHGPLITFHLNLTQGNAVVRYSSKEEAAKAQKSLHMCVLGNTTILAEFAGEEEVNRFLAQGQALPPTSSWQSSSASSQPRLSAAGSSHGLVRSDAGHWNAPCLGGKGSSELLWGGVPQYSSSLWGPPSADDSRVIGSPTPLTTLLPGDLLSGESL

SEQ ID NO: 8 - protein, 860 aa, Drosophila melanogaster
MREALFSQDGWGCQHVNQDTNWEVPSSPEPANKDAPGPPMWKPSINNGTDLWESNLRNGGQPAAQQVPKPSWGHTPSSNLGGTWGEDDDGADSSSVWTGGAVSNAGSGAAVGVNQAGVNVGPGGVVSSGGPQWGQGVVGVGLGSTGGNGSSNITGSSGVATGSSGNSSNAGNGWGDPREIRPLGVGGSMDIRNVEHRGGNGSGATSSDPRDIRMIDPRDPIRGDPRGISGRLNGTSEMWGHHPQMSHNQLQGINKMVGQSVATASTSVGTSGSGIGPGGPGPSTVSGNIPTQWGPAQPVSVGVSGPKDMSKQISGWEEPSPPPQRRSIPNYDDGTSLWGQQTRVPAASGHWKDMTDSIGRSSHLMRGQSQTGGIGIAGVGNSNVPVGANPSNPISSVVGPQARIPSVGGVQHKPDGGAMWVHSGNVGGRNNVAAVTTWGDDTHSVNVGAPSSGSVSSNNWVDDKSNSTLAQNSWSDPAPVGVSWGNKQSKPPSNSASSGWSTAAGVVDGVDLGSEWNTHGGIIGKSQQQQKLAGLNVGMVNVINAEIIKQSKQYRILVENGFKKEDVERALVIANMNIEEAADMLRANSSLSMDGWRRHDESLGSYADHNSSTSSGGFAGRYPVNSGQPSMSFPHNNLMNNMGGTAVTGGNNNTNMTALQVQKYLNQGQHGVAVGPQAVGNSSAVSVGFGQNTSNAAVAGAASVNIAANTNNQPSGQQIRMLGQQIQLAIHSGFISSQILTQPLTQTTLNLLNQLLSNIKHLQAAQQSLTRGGNVNPMAVNVAISKYKQQIQNLQNQINAQQAVYVKQQNMQPTSQQQQPQQQQLPSVHLSNSGNDYLRGHDAINNLQSNFSELNINKPS

SEQ ID NO: 9 - protein, 1213 aa, Homo sapiens
MDADSASSSESERNITIMASGNTGGEKDGLRNSTGLGSQNKFVVGSSSNNVGHGSSTGPWGFSHGAIISTCQVSVDAPESKSESSNNRMNAWGTVSSSSNGGLNPSTLNSASNHGAWPVLENNGLALKGPVGSGSSGINIQCSTIGQMPNNQSINSKVSGGSTHGTWGSLQETCESEVSGTQKVSFSGQPQNITTEMTGPNNTTNFMTSSLPNSGSVQNNELPSSNTGAWRVSTMNHPQMQAPSGMNGTSLSHLSNGESKSGGSYGTTWGAYGSNYSGDKCSGPNGQANGDTVNATLMQPGVNGPMGTNFQVNTNKGGGVWESGAANSQSTSWGSGNGANSGGSRRGWGTPAQNTGTNLPSVEWNKLPSNQHSNDSANGNGKTFTNGWKSTEEEDQGSATSQTNEQSSVWAKTGGTVESDGSTESTGRLEEKGTGESQSRDRRKIDQHTLLQSIVNRTDLDPRVLSNSGWGQTPIKQNTAWDTETSPRGERKTDNGTEAWGSSATQTFNSGACIDKTSPNGNDTSSVSGWGDPKPALRWGDSKGSNCQGGWEDDSAATGMVKSNQWGNCKEEKAAWNDSQKNKQGWGDGQKSSQGWSVSASDNWGETSRNNHWGEANKKSSSGGSDSDRSVSGWNELGKTSSFTWGNNINPNNSSGWDESSKPTPSQGWGDPPKSNQSLGWGDSSKPVSSPDWNKQQDIVGSWGIPPATGKPPGTGWLGGPIPAPAKEEEPTGWEEPSPESIRRKMEIDDGTSAWGDPSKYNYKNVNMWNKNVPNGNSRSDQQAQVHQLLTPASAISNKEASSGSGWGEPWGEPSTPATTVDNGTSAWGKPIDSGPSWGEPIAAASSTSTWGSSSVGPQALSKSGPKSMQDGWCGDDMPLPGNRPTGWEEEEDVEIGMWNSNSSQELNSSLNWPPYTKKMSSKGLSGKKRRRERGMMKGGNKQEEAWINPFVKQFSNISFSRDSPEENVQSNKMDLSGGMLQDKRMEIDKHSLNIGDYNRTVGKGPGSRPQISKESSMERNPYFDKDGIVADESQNMQFMSSQSMKLPPSNSALPNQALGSIAGLGMQNLNSVRQNGNPSMFGVGNTAAQPRGMQQPPAQPLSSSQPNLRAQVPPPLLSPQVPVSLLKYAPNNGGLNPLFGPQQVAMLNQLSQLNQLSQISQLQRLLAQQQRAQSQRSVPSGNRPQQDQQGRPLSVQQQMMQQSRQLDPNLLV

SEQ ID NO: 10 - protein, 1223 aa, Homo sapiens
MREKEQEREEQLMEDKKRKKEDKKKKEATQKVTEQKTKVPEVTKPSLSQPTAASPIGSSPSPPVNGGNNAKRVAVPNGQPPSAARYMPREVPPRFRCQQDHKVLLKRGQPPPPSCMLLGGGAGPPPCTAPGANPNNAQVTGALLQSESGTAPDSTLGGAAASNYANSTWGSGASSNNGTSPNPIHIWDKVIVDGSDMEEWPCIASKDTESSSENTTDNNSASNPGSEKSTLPGSTTSNKGKGSQCQSASSGNECNLGVWKSDPKAKSVQSSNSTTENNNGLGNWRNVSGQDRIGPGSGFSNFNPNSNPSAWPALVQEGTSRKGALETDNSNSSAQVSTVGQTSREQQSKMENAGVNFVVSGREQAQIHNTDGPKNGNTNSLNLSSPNPMENKGMPFGMGLGNTSRSTDAPSQSTGDRKTGSVGSWGAARGPSGTDTVSGQSNSGNNGNNGKEREDSWKGASVQKSTGSKNDSWDNNNRSTGGSWNFGPQDSNDNKWGEGNKMTSGVSQGEWKQPTGSDELKIGEWSGPNQPNSSTGAWDNQKGHPLPENQGNAQAPCWGRSSSSTGSEVGGQSTGSNHKAGSSDSHNSGRRSYRPTHPDCQAVLQTLLSRTDLDPRVLSNTGWGQTQIKQDTVWDIEEVPRPEGKSDKGTEGWESAATQTKNSGGWGDAPSQSNQMKSGWGELSASTEWKDPKNTGGWNDYKNNNSSNWGGGRPDEKTPSSWNENPSKDQGWGGGRQPNQGWSSGKNGWGEEVDQTKNSNWESSASKPVSGWGEGGQNEIGTWGNGGNASLASKGGWEDCKRSPAWNETGRQPNSWNKQHQQQQPPQQPPPPQPEASGSWGGPPPPPPGNVRPSNSSWSSGPQPATPKDEEPSGWEEPSPQSISRKMDIDDGTSAWGDPNSYNYKNVNLWDKNSQGGPAPREPNLPTPMTSKSASDSKSMQDGWGESDGPVTGARHPSWEEEEDGGVWNTTGSQGSASSHNSASWGQGGKKQMKCSLKGGNNDSWMNPLAKQFSNMGLLSQTEDNPSSKMDLSVGSLSDKKFDVDKRAMNLGDFNDIMRKDRSGFRPPNSKDMGTTDSGPYFEKGGSHGLFGNSTAQSRGLHTPVQPLNSSPSLRAQVPPQFISPQVSASMLKQFPNSGLSPGLFNVGPQLSPQQIAMLSQLPQIPQFQLACQLLLQQQQQQQLLQNQRKISQAVRQQQEQQLARMVSALQQQQQQQQRQPGM

SEQ ID NO: 11 - protein, 1214 aa, Homo sapiens
MATGSAQGNFTGHTKKTNGNNGTNGALVQSPSNQSALGAGGANSNGSAARVWGVATGSSSGLAHCSVSGGDGKMDTMIGDGRSQNCWGASNSNAGINLNLNPNANPAAWPVLGHEGTVATGNPSSICSPVSAIGQNMGNQNGNPTGTLGAWGNLLPQESTEPQTSTSQNVSFSAQPQNLNTDGPNNTNPMNSSPNPINAMQTNGLPNWGMAVGMGAIIPPHLQGLPGANGSSVSQVSGGSAEGISNSVWGLSPGNPATGNSNSGFSQGNGDTVNSALSAKQNGSSSAVQKEGSGGNAWDSGPPAGPGILAWGRGSGNNGVGNIHSGAWGHPSRSTSNGVNGEWGKPPNQHSNSDINGKGSTGWESPSVTSQNPTVQPGGEHMNSWAKAASSGTTASEGSSDGSGNHNEGSTGREGTGEGRRRDKGIIDQGHIQLPRNDLDPRVLSNTGWGQTPVKQNTAWEFEESPRSERKNDNGTEAWGCAATQASNSGGKNDGSIMNSTNTSSVSGWVNAPPAAVPANTGWGDSNNKAPSGPGVWGDSISSTAVSTAAAAKSGHAWSGAANQEDKSPTWGEPPKPKSQHWGDGQRSNPAWSAGGGDWADSSSVLGHLGDGKKNGSGWDADSNRSGSGWNDTTRSGNSGWGNSTNTKANPGTNWGETLKPGPQQNWASKPQDNNVSNWGGAASVKQTGTGWIGGPVPVKQKDSSEATGWEEPSPPSIRRKMEIDDGTSAWGDPSNYNNKTVNMWDRNNPVIQSSTTTNTTTTTTTTTSNTTHRVETPPPHQAGTQLNRSPLLGPGRKVSSGWGEMPNVHSKTENSWGEPSSPSTLVDNGTAAWGKPPSSGSGWGDHPAEPPVAFGRAGAPVAASALCKPASKSMQEGWGSGGDEMNLSTSQWEDEEGDVWNNAASQESTSSCSSWGNAPKKGLQKGMKTSGKQDEAWIMSRLIKQLTDMGFPREPAEEALKSNNMNLDQAMSALLEKKVDVDKRGLGVTDHNGMAAKPLGCRPPISKESSVDRPTFLDKDGGLVEEPTPSPFLPSPSLKLPLSHSALPSQALGGIASGLGMQNLNSSRQIPSGNLGMFGNSGAAQARTMQQPPQPPVQPLNSSQPSLRAQVPQFLSPQVQAQLLQFAAKNIGLNPALLTSPINPQHMTMLNQLYQLQLAYQRLQIQQQMLQAQRNVSGSMRQQEQQVARTITNLQQQIQQHQRQLAQALLVKQ

SEQ ID NO: 12 - DNA, 5012 bp, Drosophila melanogaster
cgtccccatcgtcgaacgtgcgcagaaatttatattcaattacatcgaattataattattgttgagtctaatcagttttcgcattcaagattgttattaatcccagtttattttgtgaaatatgaaaaactataattgattgtcgaatgtgtgtatgttttacaacacatacacactttgtgaactcgtcaaagtggcttgacatgcatacacacaaaaatgcacatatatttacatatatagagaaaatcaacttctcgttcagtaaaggcaaaaattgttgcaaatattcggtgaactattagcgtatacacaagtgttcaattttactataaacgcatattctggcaatagatattaatttctaatttctgtttgaacacggaaagaacaaggattcagattaagtcattccttttcgattaaattaaataacttttaaaaattgccataaatgcttatgataatgaagttaaagatcaagtggatgttggaatagtaaggaatgctacttttgaagctgacaatcagttagaacacttgagcactatgcgtgaagcccttttttcccaagatggctggggctgtcagcatgttaaccaggatactaattgggaagttcccagttcgccagaaccagccaataaggatgcacccggtccaccaatgtggaagccaagcattaacaatggtactgatctttgggagtccaatttgagaaacggaggtcagccggccgcacagcaagttccaaagccgtcgtggggtcatacaccatcctctaacttaggtggaacatggggtgaggacgacgatggcgccgatagtagtagtgtgtggactggaggagctgttagcaacgcgggatccggagctgcagtgggagtaaaccaagccggagttaatgtcggtccaggcggtgttgtttcgtctggcggacctcagtggggacaaggtgtcgttggcgtcggacttggatcaactggaggtaacgggtcaagcaatataactggatcgtctggagtcgcaacaggtagtagcggaaactccagcaacgctggtaacggttggggagaccctcgtgaaatacgccctttgggagttggtggctccatggatattcgaaatgttgaacatcgcggcggtaacggttctggagcaacttcgagcgatccacgagacattcgcatgatcgatccgcgtgaccctattcgaggagatccccgtggaatatctggtcgtcttaatgggacctctgaaatgtggggtcatcatccacaaatgtcccataaccagttgcaaggtatcaacaaaatggttggtcaaagtgtagcaactgccagcaccagtgtcggaacatctggctcgggcatcggtcctggaggtcccggtcctagtacagtatcaggcaatatcccaacacagtgggggcctgctcaaccggtaagcgttggtgtaagtggtcccaaagacatgtcaaaacagataagtggatgggaggaaccatcaccaccgcctcagcgtcgcagtattcctaactacgatgatggtacatcgttgtggggtcagcaaactcgtgttcccgctgcaagcggtcactggaaagacatgactgattcgataggtcgtagtagtcatctcatgcgtggccaaagccaaacgggaggtataggaatagccggcgttggaaatagcaatgttccagtgggagccaatccaagtaatcctataagcagtgtagttggacctcaagcccggattccatctgtgggcggcgtacaacacaaaccagacggcggcgctatgtgggtgcattccggcaatgtaggtggcagaaataatgttgctgctgttactacttggggagatgacactcatagcgttaatgtcggcgctcccagcagtggcagtgtatccagcaacaattgggttgatgacaagtccaactcaaccttggcacaaaactcttggagcgacccggcccctgttggagttagttggggcaataagcaaagcaaaccgccaagcaatagtgcttcatcaggttggagcactgctgcgggcgtggtggatggggttgatctaggatctgagtggaacacgcacggggggattattggaaaatctcagcaacaacaaaaactagcgggacttaacgtgggaatggtgaacgtaattaacgcggagatcattaagcaaagcaagcaatacaggatccttgtcgagaacggctttaaaaaggaagatgtagagcgggcattagtgattgctaatatgaacatcgaagaggcagccgatatgctccgtgccaactcatccctatcaatggatggttggcgtcgacatgatgagtcccttggatcttatgccgaccacaatagttcaacaagcagcggtggatttgctggtcgttacccggtcaacagtggacaaccttcaatgtcctttcctcataataaccttatgaataacatgggaggtaccgctgttactggaggtaacaacaatacaaacatgacagctttacaggtgcaaaagtatttaaatcaagggcaacatggtgtcgctgttggaccgcaagccgttggtaattcttcagcagtatctgtcggatttggtcagaacacgtctaacgcagcagtggcaggagcagcctctgtaaatatagcagcaaatacaaacaaccaaccgtctggtcagcaaattcgcatgctaggccagcaaattcagttggccattcatagtggtttcatatctagtcagatattgactcaaccgctaactcaaacaacccttaaccttttaaaccaacttcttagcaatattaagcatctccaggctgcgcagcaatcccttacccgcgggggaaatgtcaatccaatggcagtgaatgtggctatatctaaatacaagcagcaaatccagaatttacagaaccagataaatgcacaacaggctgtgtatgtaaaacagcaaaatatgcaaccaacttcacaacaacaacagccccaacaacagcaacttccttctgttcatctaagtaactcaggcaacgactatttaagaggtcacgatgcaataaataatttgcaaagcaacttttctgagctcaatattaataagccaagtggatatcaaggagcgtccaatcaacaatcccgattaaatcagtggaagcttccagtattagataaggagatcaactctgacagtacggaattttctcgtgccccaggtgcaacgaaacaaaatttgacggccaacacaagcaacataaactctttgggtcttcaaaacgatagtacatggtcaactggacgcagtattggtgacggttggcctgatccctcatctgataacgagaataaagactggtctgttgctcagccaacttcagcagcaactgcttacactgatctggtccaagagtttgagccaggcaagccatggaagggttcacagatcaaaagcatagaagatgatcccagcattacaccaggaagcgttgctagatctccattgtctattaattcgacgccaaaagatgctgacatatttgccaataccggtaaaaattcaccgactgatttaccgccactaagtttatcgtcgtctacatggagttttaatccaaaccaaaattatccgagtcacagttggtctgacaatagtcaacaatgtaccgccacttcggagctttggacaagcccgctaaataaatcatcgtctcgaggtcccccgccaggattgactgccaattcaaataaatctgcaaatagtaatgcgtcaacgccaacaactattaccggaggtgcgaatggatggttacagcctcgaagtggcggtgttcaaaccacaaacactaattggacaggtggtaacaccacttggggctccagttggttgcttttgaaaaatctaacagcacagattgatggtcctactttgcgtacactgtgtatgcagcatgggccccttgtcagctttcacccgtatttgaaccaaggaattgccttatgtaaatatactactcgtgaggaggcgaacaaggcgcaaatggcgttaaacaactgtgtcctcgccaacaccacaatatttgctgaatctcccagcgagaacgaggtgcaaagcattatgcagcacttaccacaaactccttcctctacaagctctagtggaactagtggtggcaacgtcggaggcgtcggcacttcagccaataatgcaaacagtggttctgcagcttgtctgtccggaaacaatagcggcaacggaaacggcagcgcgagcggcgccggcagcggcaacaatggcaacagtagctgcaacaacagtgccgccggggggggcagcagcagcaacaacacgattaccactgtagcaaattcgaatcttgttggttctagtggctctgtctcaaattcctctggcgttactgctaactctagtactgtttctgtagttagttgtacagcgagtgggaattccataaatggggcaggtactgcaaacagttctggttcaaagagtagtgcaaacaatttagctagcggccagtctagcgcttctaacttaactaatagcaccaattcaacatggcgacaaactagccaaaaccaagctcttcaaagtcaaagcaggccatcaggcagagaagctgactttgattatatatctctcgtttattccattgttgatgattaaaagatcaattaccagttccattggtcattggccattgactatcgcattctgtgactcaagacacacacacccaaaagcttcaaattatatgatactaagtatgtatgaaaagcaagaccatgttgtgaggaatacaaaagtggatcgactgatagacgaatggacttagaattttgtatgcctgtagatttatttttcttcatcgttcatagatataacttcgatatgtaagtttaaatttaaccaatataaaaaaacatccaacatacatatgtatgtatatgtcaaaaaaaaaaaaaaaaaaaaaa

SEQ ID NO: 13 - DNA, 3642 bp, Homo sapiens
atggatgctgattctgcctccagttctgaatcagagagaaacatcactatcatggcttcagggaacacaggtggtgaaaaagatggccttcggaatagcactggacttggttcccaaaacaagtttgtagttggtagcagcagcaataatgtgggccatggaagtagtactgggccatggggtttttcccatggagccataataagcacatgtcaggtctctgtggatgctcctgaaagcaaatctgaaagtagcaacaatagaatgaatgcttggggcactgtaagttcttcatcaaatggagggttaaatccaagcactttgaattcagctagcaaccatggtgcctggccagtattagagaacaatggacttgccctaaaagggcctgtagggagtggtagttctggcattaatattcagtgcagtactataggccagatgcctaacaatcagagtattaactctaaagtgagtggtggttctacccatggtacctggggaagccttcaggaaacttgtgaatctgaagtaagtggtacacagaaggtttcattcagtggtcaacctcaaaatattaccactgaaatgactggaccaaataacactactaactttatgacctctagtttaccaaactccggttcagtgcagaataatgagctgcctagtagtaacacaggggcctggcgtgtgagcacaatgaatcatcctcagatgcaggctccatcaggtatgaatggcacttccctttctcaccttagcaatggagagtcaaaaagtggaggctcttatggtactacatggggtgcctatggttctaattactctggagacaaatgttcaggccctaatggccaagctaatggtgacactgtgaatgcaactctaatgcagcctggcgtaaatggtcctatgggcactaactttcaagttaacacaaacaaaggaggtggtgtgtgggaatctggtgcagcaaactcccagagtacatcatggggaagtggaaatggcgcaaattctggaggaagtcgaagaggatggggaacccctgcacaaaacactggcactaatttacccagcgttgagtggaacaaactgcctagcaatcagcattccaatgatagtgcaaatggcaatggtaagacgtttacaaatggatggaaatctactgaggaagaggatcagggttctgccacatctcagacaaatgagcaaagcagtgtgtgggccaaaacaggaggtacagtggagagcgatggtagtacagaaagcactggacgccttgaggaaaaaggaactggggaaagtcagagtagagacagaagaaaaattgatcagcacacattactccaaagcattgtaaacagaactgacttagatccacgtgtcctgtccaactctggttggggacagactcctattaagcagaatactgcctgggatacagaaacatcacctagaggggaacgaaagactgacaatgggacagaggcctggggaagctctgcaacacagacttttaactcaggggcatgtatagataagactagccctaatggtaatgatacctcatctgtatcagggtggggcgatcccaaacctgctctgaggtggggagattccaaaggctcaaactgccagggggggtgggaagatgattctgctgctacaggaatggtcaagagcaatcagtgggggaattgcaaagaggagaaggctgcatggaatgactcgcaaaagaataaacagggatggggtgatggacaaaaatcaagccaagggtggtctgtttctgccagtgataactggggagaaacttcaaggaataaccattggggtgaggccaataagaaatccagctcaggaggtagtgacagtgacaggtccgtttccggttggaacgaacttggtaaaactagttctttcacttggggaaacaacataaatccaaataattcatcaggatgggatgaatcttctaaacctactccttcccagggatggggagaccctccaaagtctaatcagtctctaggttggggagattcgtcaaagccagtcagctctccagactggaacaagcaacaagacattgttggatcttggggaatcccaccagctacaggcaaacctcctggtacaggctggctggggggacctataccagccccagcaaaagaagaagaacccacaggctgggaggaaccatccccagaatctatacgtcgcaaaatggagattgatgatggaacttcagcttggggagatccaagcaaatacaactacaaaaatgtgaacatgtggaacaaaaacgtcccaaatggcaacagccgttcagaccagcaagcacaggtacatcagctgctaacgcctgcaagtgccatctcaaacaaagaggcaagcagtggctctggctggggtgagccctggggggagccttctactccagccacaactgtggataatggtacttcagcatggggtaagcccatagacagtggtcccagctggggggaacccattgctgcggcatccagcacatccacgtggggctccagctctgttggtccacaagcattaagcaaatctgggccaaaatctatgcaagatggctggtgtggtgatgatatgccattgcctggaaatcgccccactggctgggaagaggaagaggatgtggagattggaatgtggaatagtaattcatctcaagagcttaactcatctttaaattggccaccatatacaaagaaaatgtcatcgaagggtctgagtggcaaaaaaaggagaagggaaaggggaatgatgaaaggtggaaacaaacaagaagaagcgtggataaatccatttgttaaacagttttcaaacatcagtttttcgagagactcaccagaggaaaatgtacaaagcaataagatggacctttctggaggaatgttacaagacaaacgaatggagatagataaacatagcctaaatattggtgattacaatcgaacggtcgggaaaggccctggttctcggcctcagatttccaaagagtcttccatggagcgcaatccttattttgataaggatggcattgtagcagatgaatcccaaaacatgcagtttatgtccagtcaaagcatgaagcttcccccttcaaatagtgcactacctaaccaggcccttggctccatagcagggctgggtatgcaaaacttgaattctgttagacagaatggcaatcccagtatgtttggtgttggaaacacagcagcacaaccccggggcatgcagcagcctccagcacaacctcttagttcatctcagcctaatctccgtgctcaagtgcctcctccattactctcccctcaggttccagtttcattgctgaagtatgcaccaaacaacggtggcctgaatccactctttggccctcaacaggtagccatgctgaaccagctatcccagctaaaccagctttctcagatctcccagttacagcgattgttagcgcagcagcaaagggcgcagagtcagagaagcgtgccttctgggaaccggccgcagcaagaccagcagggtcgacctcttagtgtgcagcagcaaatgatgcaacaatctcgtcaacttgatccaaacctgttggtgtag

SEQ ID NO: 14 - DNA, 3672 bp, Homo sapiens
atgagagagaaggagcaagaaagggaagaacagttaatggaagacaagaaaaggaagaaagaggataaaaagaaaaaagaagccactcagaaggtcacggaacaaaaaaccaaagtgcccgaagtgacgaaaccaagtttaagccaaccaacggccgccagcccaattggcagctctccatcgccaccagtcaatggtggcaacaatgccaaaagggtggcagtgccgaacggacaaccgccaagcgccgcccgctacatgcctcgggaggtgccgccgcgattccgttgccagcaggaccacaaagtgttactaaaacgtgggcagccccctccaccgtcctgcatgctccttgggggtggggcagggcctcctccctgcacagcacctggagcaaacccaaacaacgcacaagtgacaggagcgctgctgcagagtgagagtgggactgcgccagactcaacccttggaggtgctgctgcttcaaattatgcaaattccacttggggctcgggagcctcctccaacaacggcacctcccccaacccaattcacatctgggacaaggtgattgtagacgggtctgacatggaagagtggccttgtattgccagcaaagacactgaatcttcttccgaaaacaccaccgataacaacagtgcctcgaaccctggctctgagaagagcactctgccaggaagcaccactagtaacaaaggaaaagggagccagtgccagtctgcaagttctgggaacgaatgtaatcttggggtctggaaatctgaccctaaggctaaatctgttcaatcttccaactctactacagagaacaacaatggactaggaaattggaggaatgtgagtggtcaggatagaattggacctggctctggcttcagcaactttaacccaaatagcaacccatctgcctggccagcactggtccaagaaggaacttctaggaaaggggcattggaaacagataatagtaattccagtgcacaggttagcacagtaggtcagacatccagggaacagcagtcaaagatggaaaatgcgggtgttaattttgttgtctctggcagagaacaggctcaaattcataacactgatggaccaaaaaatggaaacactaactccttgaacttaagttcaccaaaccccatggagaataagggaatgccctttggaatgggcttggggaacacctccaggagcactgatgccccttcacaaagcactggagatcgaaagactgggagtgttggatcttggggtgcagctagggggccttctggaactgacacagtctctggacaaagcaattctggaaacaatgggaacaatggaaaagagagagaggactcctggaaaggagcttctgttcagaaatcaactgggtcaaaaaatgactcttgggacaacaataacaggtctacgggtgggtcctggaactttggcccccaggactctaatgacaacaaatggggtgaagggaacaaaatgacatctggggtctctcagggagaatggaaacagccgactgggtctgatgagttgaaaattggagaatggagtggtccaaaccaaccaaattctagcactggagcatgggacaatcaaaagggccaccccctccctgaaaaccaaggcaatgcccaggctccctgttggggaagatcttccagctccacaggaagtgaagttggaggtcaaagcactggaagcaaccacaaagcaggaagtagtgacagtcataactctggccgtcggtcgtacaggcccacacatcctgattgtcaggctgtcttgcagactcttttgagccgaactgatttggaccccagggtgctctcaaacactggctggggccaaactcaaattaagcaggacacagtgtgggacattgaagaggtgccaaggcctgaggggaaatctgacaaaggaactgaggggtgggagagcgctgccacacagaccaagaactcagggggctggggagatgcacccagccaaagcaatcaaatgaagtctggatggggggagctctcagcctctacagagtggaaagaccccaagaacacaggaggctggaatgactacaagaacaacaactcttccaactggggaggaggacgacctgatgaaaagacaccttcctcttggaatgagaatcccagcaaggatcaggggtggggaggtggacgccagcccaatcaaggatggtcttctggaaagaatggttggggggaggaagtcgatcagacaaaaaacagcaattgggaaagttctgcaagtaaacctgtgtctgggtggggtgaaggagggcagaatgaaatcgggacttggggtaatggtggcaatgcaagcctagcttcaaaaggtgggtgggaggattgcaaaagatccccagcatggaatgagacgggccgacagcccaattcctggaataaacaacaccaacagcagcagcccccacagcagccgccgccaccacaaccagaggcttctggttcgtggggaggcccacccccaccacctccaggcaacgttcgaccttccaattccagctggagcagcgggccacagcctgcaacacctaaggatgaggaacccagtggttgggaagagccatccccacagtcaattagtcggaaaatggacattgatgatggcacttcagcatggggagaccctaacagttataactacaagaatgtgaatctgtgggataagaattcccaagggggcccagcacctcgagaaccaaacctgcccaccccaatgaccagtaaatcggcatcagattccaaatctatgcaagacggctggggggagagtgacgggccagtcacaggagctcgccatcccagctgggaagaggaggaggatggaggagtctggaacaccactggctctcagggcagtgcttcctcccacaactcagcaagctggggacaaggaggaaagaaacaaatgaagtgctcactcaaaggaggaaacaatgattcatggatgaatcctcttgccaaacagttttcaaatatgggattgctgagtcagactgaagataatccaagcagcaaaatggatttgtctgtaggaagcctttcagataaaaaatttgatgtggacaagcgagcgatgaatctcggggattttaatgatatcatgaggaaggatcgatctgggttccgtccacctaattccaaagacatgggaaccacagatagtgggccttattttgagaagggcggtagtcatggtttgtttggaaacagcacagcacaatcgagaggtctgcacacacccgtgcagccactaaattcttctcccagtctccgggcgcaagtgcctccccagtttatttccccccaggtttctgcctcaatgctcaagcagtttcccaacagtggcctgagtccaggtcttttcaatgtggggccccagttatctcctcaacaaattgccatgctgagccagcttccacaaattccccagtttcagttggcatgtcagcttctcttgcagcagcagcaacagcagcagttgttacagaaccagagaaagatttctcaagctgtacgccaacagcaagagcagcagctggctcgaatggtgagtgcactgcagcagcagcagcagcagcagcagaggcagccaggcatgtag

SEQ ID NO: 15 - DNA, 3645 bp, Homo sapiens
atggctacagggagtgcccagggcaacttcactggacataccaagaagacaaatggcaataatggcaccaatggcgcactcgtccaaagcccttctaatcagagtgcccttggagcagggggagcgaacagtaatggaagtgcggccagagtgtggggtgtagccacaggctccagctctggcctggctcactgctctgtcagtggtggggatggaaaaatggacactatgattggagatgggagaagtcagaattgctggggtgcttccaactccaatgctggcattaatcttaaccttaatcctaatgccaacccagctgcctggcctgtacttggacatgaaggaaccgtggcgacaggcaacccttccagtatttgcagtccagtcagtgccataggtcaaaatatgggcaaccagaacgggaacccaacaggcactttaggtgcttggggaaacttgctgccacaagagagcacagaaccacaaacgtccacttctcagaatgtgtctttcagcgcacaacctcagaaccttaacactgatggaccaaataacactaaccccatgaactcttcacccaaccctatcaatgcaatgcagacaaatggactgccaaactggggcatggctgttggtatgggggccatcatcccgccccacctgcaaggccttcctggtgctaatggatcatcagtttctcaagtcagtgggggcagtgctgaaggaataagcaattctgtgtggggactgtccccaggtaaccctgccacaggaaatagcaattctgggttcagtcaggggaatggagacactgtgaactcagcattaagtgctaaacaaaatggatccagcagtgctgtgcaaaaggaaggaagtggaggaaatgcttgggattcaggacctcctgctggtcctggaatactcgcctggggaaggggcagtggcaacaatggcgttggtaatatccattcaggagcttggggccaccccagccgaagcacctctaacggtgtgaatggggaatggggaaagcccccaaaccagcattccaacagtgacatcaatgggaaaggatcaacagggtgggagagtcctagtgtcaccagccagaaccctaccgtacagcctggtggtgaacacatgaactcctgggccaaagcggcatcttctggaactacagcaagtgaaggaagtagtgatggttctggcaaccacaatgaaggaagcactgggagggaaggaacgggagaaggccgaaggcgagataaagggattatagaccaagggcacatccagttgccaaggaatgatcttgacccaagagttctgtctaatactggttggggacagactcctgtaaagcaaaacactgcctgggaatttgaagaatcccctaggtctgaaaggaaaaatgacaatgggacagaggcctggggttgtgcagctactcaggcttcaaactcaggggggaagaacgatgggtccatcatgaacagtacaaatacctcttcagtatctgggtgggtcaacgcgccacctgccgctgtgccagcaaacacaggttggggagacagcaacaacaaagcgccaagtggcccgggggtttggggggactcgataagctctactgctgttagtactgctgctgctgccaagagtggccatgcttggagtggggccgcaaatcaggaggacaagtcacccacctggggtgagcctccaaagcccaaatcccaacactggggagatggacaaagatcaaatccagcctggagtgcaggagggggagattgggcagattcatcgtctgtccttggacacttgggggatgggaaaaaaaatggatctggatgggatgctgacagtaataggtcagggtctggttggaatgacaccacgagatctgggaacagtggctggggcaacagcacaaatacaaaggccaatccaggtacaaactggggggagactttaaaacctggcccccaacagaactgggctagcaaaccccaagacaacaatgtgagtaactggggaggagctgcttctgtgaaacagacaggaacagggtggatcggggggccggtaccggtcaaacagaaggacagcagtgaagcaactggctgggaagaaccctctccaccgtccattcgccgcaaaatggaaattgatgatggtacctcagcttggggggacccaagcaactataacaataaaactgtaaacatgtgggatagaaacaacccggtcatccagagcagtaccacgaccaataccaccaccaccaccaccactaccacgagcaacaccacacacagggtcgagacgccgcccccgcaccaggctggtactcagctgaatcgatcaccgttgcttggtccaggtaggaaagtttcatcaggctggggagaaatgcctaatgttcactcaaagactgaaaactcttggggagaaccatcctccccttctaccctggtggataatggcacagcagcatgggggaagccacccagcagtggcagcgggtggggagatcaccctgccgagccgccggtggcatttggaagagctggcgcacctgttgctgcctcagccctgtgcaaaccagcttcaaaatctatgcaagaaggctggggcagtggtggggatgaaatgaacctcagtaccagccagtgggaggatgaagaaggggacgtgtggaataatgctgcttcccaagaaagcacctcctcctgcagctcctgggggaacgcccccaaaaaaggacttcaaaagggcatgaagacgtctggcaagcaggatgaggcctggatcatgagccggctgatcaaacaactcacagacatgggcttcccgagagagccagctgaggaggccttgaagagtaacaatatgaatcttgatcaggccatgagcgctctgctggaaaagaaggtggacgtggacaagcgtgggctgggagtgaccgaccataatggaatggccgccaagcccctcggctgccgcccgccaatctccaaagagtcttccgtggaccgccccacctttcttgacaaggatggcggcctcgtggaagagcccacgccttcaccgttcttgccttccccaagcctgaagctccccctttcacacagtgcactccccagtcaggccctgggtgggattgcctccgggctgggcatgcaaaacttgaattcttctagacagataccgagtggcaatctgggtatgtttggcaatagtggagcagcacaagccaggaccatgcagcagccgccacagccaccagtgcagcctcttaactcttcccagcccagtctccgtgctcaagtgcctcagtttctatcccctcaggttcaagcacagcttttgcagtttgcagcaaaaaacattggtctcaaccctgcactattaacctcgccaattaatcctcaacatatgacgatgttgaaccagctctatcagctgcagctggcataccaacgtttacaaatccagcagcagatgttacaggcccagcgtaatgtgtccggatccatgagacaacaggagcagcaagttgcgcgcacaatcactaatctgcagcagcagatccagcagcaccagcgccagctggcccaggccctgctcgtgaagcagtag

SEQ ID NO: 16 - DNA, 38087 bp, Artificial Sequence
  Adenoviral vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacatggatgctgattctgcctccagttctgaatcagagagaaacatcactatcatggcttcagggaacacaggtggtgaaaaagatggccttcggaatagcactggacttggttcccaaaacaagtttgtagttggtagcagcagcaataatgtgggccatggaagtagtactgggccatggggtttttcccatggagccataataagcacatgtcaggtctctgtggatgctcctgaaagcaaatctgaaagtagcaacaatagaatgaatgcttggggcactgtaagttcttcatcaaatggagggttaaatccaagcactttgaattcagctagcaaccatggtgcctggccagtattagagaacaatggacttgccctaaaagggcctgtagggagtggtagttctggcattaatattcagtgcagtactataggccagatgcctaacaatcagagtattaactctaaagtgagtggtggttctacccatggtacctggggaagccttcaggaaacttgtgaatctgaagtaagtggtacacagaaggtttcattcagtggtcaacctcaaaatattaccactgaaatgactggaccaaataacactactaactttatgacctctagtttaccaaactccggttcagtgcagaataatgagctgcctagtagtaacacaggggcctggcgtgtgagcacaatgaatcatcctcagatgcaggctccatcaggtatgaatggcacttccctttctcaccttagcaatggagagtcaaaaagtggaggctcttatggtactacatggggtgcctatggttctaattactctggagacaaatgttcaggccctaatggccaagctaatggtgacactgtgaatgcaactctaatgcagcctggcgtaaatggtcctatgggcactaactttcaagttaacacaaacaaaggaggtggtgtgtgggaatctggtgcagcaaactcccagagtacatcatggggaagtggaaatggcgcaaattctggaggaagtcgaagaggatggggaacccctgcacaaaacactggcactaatttacccagcgttgagtggaacaaactgcctagcaatcagcattccaatgatagtgcaaatggcaatggtaagacgtttacaaatggatggaaatctactgaggaagaggatcagggttctgccacatctcagacaaatgagcaaagcagtgtgtgggccaaaacaggaggtacagtggagagcgatggtagtacagaaagcactggacgccttgaggaaaaaggaactggggaaagtcagagtagagacagaagaaaaattgatcagcacacattactccaaagcattgtaaacagaactgacttagatccacgtgtcctgtccaactctggttggggacagactcctattaagcagaatactgcctgggatacagaaacatcacctagaggggaacgaaagactgacaatgggacagaggcctggggaagctctgcaacacagacttttaactcaggggcatgtatagataagactagccctaatggtaatgatacctcatctgtatcagggtggggcgatcccaaacctgctctgaggtggggagattccaaaggctcaaactgccagggggggtgggaagatgattctgctgctacaggaatggtcaagagcaatcagtgggggaattgcaaagaggagaaggctgcatggaatgactcgcaaaagaataaacagggatggggtgatggacaaaaatcaagccaagggtggtctgtttctgccagtgataactggggagaaacttcaaggaataaccattggggtgaggccaataagaaatccagctcaggaggtagtgacagtgacaggtccgtttccggttggaacgaacttggtaaaactagttctttcacttggggaaacaacataaatccaaataattcatcaggatgggatgaatcttctaaacctactccttcccagggatggggagaccctccaaagtctaatcagtctctaggttggggagattcgtcaaagccagtcagctctccagactggaacaagcaacaagacattgttggatcttggggaatcccaccagctacaggcaaacctcctggtacaggctggctggggggacctataccagccccagcaaaagaagaagaacccacaggctgggaggaaccatccccagaatctatacgtcgcaaaatggagattgatgatggaacttcagcttggggagatccaagcaaatacaactacaaaaatgtgaacatgtggaacaaaaacgtcccaaatggcaacagccgttcagaccagcaagcacaggtacatcagctgctaacgcctgcaagtgccatctcaaacaaagaggcaagcagtggctctggctggggtgagccctggggggagccttctactccagccacaactgtggataatggtacttcagcatggggtaagcccatagacagtggtcccagctggggggaacccattgctgcggcatccagcacatccacgtggggctccagctctgttggtccacaagcattaagcaaatctgggccaaaatctatgcaagatggctggtgtggtgatgatatgccattgcctggaaatcgccccactggctgggaagaggaagaggatgtggagattggaatgtggaatagtaattcatctcaagagcttaactcatctttaaattggccaccatatacaaagaaaatgtcatcgaagggtctgagtggcaaaaaaaggagaagggaaaggggaatgatgaaaggtggaaacaaacaagaagaagcgtggataaatccatttgttaaacagttttcaaacatcagtttttcgagagactcaccagaggaaaatgtacaaagcaataagatggacctttctggaggaatgttacaagacaaacgaatggagatagataaacatagcctaaatattggtgattacaatcgaacggtcgggaaaggccctggttctcggcctcagatttccaaagagtcttccatggagcgcaatccttattttgataaggatggcattgtagcagatgaatcccaaaacatgcagtttatgtccagtcaaagcatgaagcttcccccttcaaatagtgcactacctaaccaggcccttggctccatagcagggctgggtatgcaaaacttgaattctgttagacagaatggcaatcccagtatgtttggtgttggaaacacagcagcacaaccccggggcatgcagcagcctccagcacaacctcttagttcatctcagcctaatctccgtgctcaagtgcctcctccattactctcccctcaggttccagtttcattgctgaagtatgcaccaaacaacggtggcctgaatccactctttggccctcaacaggtagccatgctgaaccagctatcccagctaaaccagctttctcagatctcccagttacagcgattgttagcgcagcagcaaagggcgcagagtcagagaagcgtgccttctgggaaccggccgcagcaagaccagcagggtcgacctcttagtgtgcagcagcaaatgatgcaacaatctcgtcaacttgatccaaacctgttggtgtaggtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaa

SEQ ID NO: 17 - DNA, 38179 bp, Artificial Sequence
  Adenoviral vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctccgcggccgcccccttcaccatgagagagaaggagcaagaaagggaagaacagttaatggaagacaagaaaaggaagaaagaggataaaaagaaaaaagaagccactcagaaggtcacggaacaaaaaaccaaagtgcccgaagtgacgaaaccaagtttaagccaaccaacggccgccagcccaattggcagctctccatcgccaccagtcaatggtggcaacaatgccaaaagggtggcagtgccgaacggacaaccgccaagcgccgcccgctacatgcctcgggaggtgccgccgcgattccgttgccagcaggaccacaaagtgttactaaaacgtgggcagccccctccaccgtcctgcatgctccttgggggtggggcagggcctcctccctgcacagcacctggagcaaacccaaacaacgcacaagtgacaggagcgctgctgcagagtgagagtgggactgcgccagactcaacccttggaggtgctgctgcttcaaattatgcaaattccacttggggctcgggagcctcctccaacaacggcacctcccccaacccaattcacatctgggacaaggtgattgtagacgggtctgacatggaagagtggccttgtattgccagcaaagacactgaatcttcttccgaaaacaccaccgataacaacagtgcctcgaaccctggctctgagaagagcactctgccaggaagcaccactagtaacaaaggaaaagggagccagtgccagtctgcaagttctgggaacgaatgtaatcttggggtctggaaatctgaccctaaggctaaatctgttcaatcttccaactctactacagagaacaacaatggactaggaaattggaggaatgtgagtggtcaggatagaattggacctggctctggcttcagcaactttaacccaaatagcaacccatctgcctggccagcactggtccaagaaggaacttctaggaaaggggcattggaaacagataatagtaattccagtgcacaggttagcacagtaggtcagacatccagggaacagcagtcaaagatggaaaatgcgggtgttaattttgttgtctctggcagagaacaggctcaaattcataacactgatggaccaaaaaatggaaacactaactccttgaacttaagttcaccaaaccccatggagaataagggaatgccctttggaatgggcttggggaacacctccaggagcactgatgccccttcacaaagcactggagatcgaaagactgggagtgttggatcttggggtgcagctagggggccttctggaactgacacagtctctggacaaagcaattctggaaacaatgggaacaatggaaaagagagagaggactcctggaaaggagcttctgttcagaaatcaactgggtcaaaaaatgactcttgggacaacaataacaggtctacgggtgggtcctggaactttggcccccaggactctaatgacaacaaatggggtgaagggaacaaaatgacatctggggtctctcagggagaatggaaacagccgactgggtctgatgagttgaaaattggagaatggagtggtccaaaccaaccaaattctagcactggagcatgggacaatcaaaagggccaccccctccctgaaaaccaaggcaatgcccaggctccctgttggggaagatcttccagctccacaggaagtgaagttggaggtcaaagcactggaagcaaccacaaagcaggaagtagtgacagtcataactctggccgtcggtcgtacaggcccacacatcctgattgtcaggctgtcttgcagactcttttgagccgaactgatttggaccccagggtgctctcaaacactggctggggccaaactcaaattaagcaggacacagtgtgggacattgaagaggtgccaaggcctgaggggaaatctgacaaaggaactgaggggtgggagagcgctgccacacagaccaagaactcagggggctggggagatgcacccagccaaagcaatcaaatgaagtctggatggggggagctctcagcctctacagagtggaaagaccccaagaacacaggaggctggaatgactacaagaacaacaactcttccaactggggaggaggacgacctgatgaaaagacaccttcctcttggaatgagaatcccagcaaggatcaggggtggggaggtggacgccagcccaatcaaggatggtcttctggaaagaatggttggggggaggaagtcgatcagacaaaaaacagcaattgggaaagttctgcaagtaaacctgtgtctgggtggggtgaaggagggcagaatgaaatcgggacttggggtaatggtggcaatgcaagcctagcttcaaaaggtgggtgggaggattgcaaaagatccccagcatggaatgagacgggccgacagcccaattcctggaataaacaacaccaacagcagcagcccccacagcagccgccgccaccacaaccagaggcttctggttcgtggggaggcccacccccaccacctccaggcaacgttcgaccttccaattccagctggagcagcgggccacagcctgcaacacctaaggatgaggaacccagtggttgggaagagccatccccacagtcaattagtcggaaaatggacattgatgatggcacttcagcatggggagaccctaacagttataactacaagaatgtgaatctgtgggataagaattcccaagggggcccagcacctcgagaaccaaacctgcccaccccaatgaccagtaaatcggcatcagattccaaatctatgcaagacggctggggggagagtgacgggccagtcacaggagctcgccatcccagctgggaagaggaggaggatggaggagtctggaacaccactggctctcagggcagtgcttcctcccacaactcagcaagctggggacaaggaggaaagaaacaaatgaagtgctcactcaaaggaggaaacaatgattcatggatgaatcctcttgccaaacagttttcaaatatgggattgctgagtcagactgaagataatccaagcagcaaaatggatttgtctgtaggaagcctttcagataaaaaatttgatgtggacaagcgagcgatgaatctcggggattttaatgatatcatgaggaaggatcgatctgggttccgtccacctaattccaaagacatgggaaccacagatagtgggccttattttgagaagggcggtagtcatggtttgtttggaaacagcacagcacaatcgagaggtctgcacacacccgtgcagccactaaattcttctcccagtctccgggcgcaagtgcctccccagtttatttccccccaggtttctgcctcaatgctcaagcagtttcccaacagtggcctgagtccaggtcttttcaatgtggggccccagttatctcctcaacaaattgccatgctgagccagcttccacaaattccccagtttcagttggcatgtcagcttctcttgcagcagcagcaacagcagcagttgttacagaaccagagaaagatttctcaagctgtacgccaacagcaagagcagcagctggctcgaatggtgagtgcactgcagcagcagcagcagcagcagcagaggcagccaggcatgtagaagggtgggcgcgccgacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaa

SEQ ID NO: 18 - DNA, 67618 bp, Artificial Sequence
  Adenoviral vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctccgcggccgcccccttcaccatggctacagggagtgcccagggcaacttcactggacataccaagaagacaaatggcaataatggcaccaatggcgcactcgtccaaagcccttctaatcagagtgcccttggagcagggggagcgaacagtaatggaagtgcggccagagtgtggggtgtagccacaggctccagctctggcctggctcactgctctgtcagtggtggggatggaaaaatggacactatgattggagatgggagaagtcagaattgctggggtgcttccaactccaatgctggcattaatcttaaccttaatcctaatgccaacccagctgcctggcctgtacttggacatgaaggaaccgtggcgacaggcaacccttccagtatttgcagtccagtcagtgccataggtcaaaatatgggcaaccagaacgggaacccaacaggcactttaggtgcttggggaaacttgctgccacaagagagcacagaaccacaaacgtccacttctcagaatgtgtctttcagcgcacaacctcagaaccttaacactgatggaccaaataacactaaccccatgaactcttcacccaaccctatcaatgcaatgcagacaaatggactgccaaactggggcatggctgttggtatgggggccatcatcccgccccacctgcaaggccttcctggtgctaatggatcatcagtttctcaagtcagtgggggcagtgctgaaggaataagcaattctgtgtggggactgtccccaggtaaccctgccacaggaaatagcaattctgggttcagtcaggggaatggagacactgtgaactcagcattaagtgctaaacaaaatggatccagcagtgctgtgcaaaaggaaggaagtggaggaaatgcttgggattcaggacctcctgctggtcctggaatactcgcctggggaaggggcagtggcaacaatggcgttggtaatatccattcaggagcttggggccaccccagccgaagcacctctaacggtgtgaatggggaatggggaaagcccccaaaccagcattccaacagtgacatcaatgggaaaggatcaacagggtgggagagtcctagtgtcaccagccagaaccctaccgtacagcctggtggtgaacacatgaactcctgggccaaagcggcatcttctggaactacagcaagtgaaggaagtagtgatggttctggcaaccacaatgaaggaagcactgggagggaaggaacgggagaaggccgaaggcgagataaagggattatagaccaagggcacatccagttgccaaggaatgatcttgacccaagagttctgtctaatactggttggggacagactcctgtaaagcaaaacactgcctgggaatttgaagaatcccctaggtctgaaaggaaaaatgacaatgggacagaggcctggggttgtgcagctactcaggcttcaaactcaggggggaagaacgatgggtccatcatgaacagtacaaatacctcttcagtatctgggtgggtcaacgcgccacctgccgctgtgccagcaaacacaggttggggagacagcaacaacaaagcgccaagtggcccgggggtttggggggactcgataagctctactgctgttagtactgctgctgctgccaagagtggccatgcttggagtggggccgcaaatcaggaggacaagtcacccacctggggtgagcctccaaagcccaaatcccaacactggggagatggacaaagatcaaatccagcctggagtgcaggagggggagattgggcagattcatcgtctgtccttggacacttgggggatgggaaaaaaaatggatctggatgggatgctgacagtaataggtcagggtctggttggaatgacaccacgagatctgggaacagtggctggggcaacagcacaaatacaaaggccaatccaggtacaaactggggggagactttaaaacctggcccccaacagaactgggctagcaaaccccaagacaacaatgtgagtaactggggaggagctgcttctgtgaaacagacaggaacagggtggatcggggggccggtaccggtcaaacagaaggacagcagtgaagcaactggctgggaagaaccctctccaccgtccattcgccgcaaaatggaaattgatgatggtacctcagcttggggggacccaagcaactataacaataaaactgtaaacatgtgggatagaaacaacccggtcatccagagcagtaccacgaccaataccaccaccaccaccaccactaccacgagcaacaccacacacagggtcgagacgccgcccccgcaccaggctggtactcagctgaatcgatcaccgttgcttggtccaggtaggaaagtttcatcaggctggggagaaatgcctaatgttcactcaaagactgaaaactcttggggagaaccatcctccccttctaccctggtggataatggcacagcagcatgggggaagccacccagcagtggcagcgggtggggagatcaccctgccgagccgccggtggcatttggaagagctggcgcacctgttgctgcctcagccctgtgcaaaccagcttcaaaatctatgcaagaaggctggggcagtggtggggatgaaatgaacctcagtaccagccagtgggaggatgaagaaggggacgtgtggaataatgctgcttcccaagaaagcacctcctcctgcagctcctgggggaacgcccccaaaaaaggacttcaaaagggcatgaagacgtctggcaagcaggatgaggcctggatcatgagccggctgatcaaacaactcacagacatgggcttcccgagagagccagctgaggaggccttgaagagtaacaatatgaatcttgatcaggccatgagcgctctgctggaaaagaaggtggacgtggacaagcgtgggctgggagtgaccgaccataatggaatggccgccaagcccctcggctgccgcccgccaatctccaaagagtcttccgtggaccgccccacctttcttgacaaggatggcggcctcgtggaagagcccacgccttcaccgttcttgccttccccaagcctgaagctccccctttcacacagtgcactccccagtcaggccctgggtgggattgcctccgggctgggcatgcaaaacttgaattcttctagacagataccgagtggcaatctgggtatgtttggcaatagtggagcagcacaagccaggaccatgcagcagccgccacagccaccagtgcagcctcttaactcttcccagcccagtctccgtgctcaagtgcctcagtttctatcccctcaggttcaagcacagcttttgcagtttgcagcaaaaaacattggtctcaaccctgcactattaacctcgccaattaatcctcaacatatgacgatgttgaaccagctctatcagctgcagctggcataccaacgtttacaaatccagcagcagatgttacaggcccagcgtaatgtgtccggatccatgagacaacaggagcagcaagttgcgcgcacaatcactaatctgcagcagcagatccagcagcaccagcgccagctggcccaggccctgctcgtgaagcagtagaagggtgggcgcgccgacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctccgcggccgcccccttcaccatggctacagggagtgcccagggcaacttcactggacataccaagaagacaaatggcaataatggcaccaatggcgcactcgtccaaagcccttctaatcagagtgcccttggagcagggggagcgaacagtaatggaagtgcggccagagtgtggggtgtagccacaggctccagctctggcctggctcactgctctgtcagtggtggggatggaaaaatggacactatgattggagatgggagaagtcagaattgctggggtgcttccaactccaatgctggcattaatcttaaccttaatcctaatgccaacccagctgcctggcctgtacttggacatgaaggaaccgtggcgacaggcaacccttccagtatttgcagtccagtcagtgccataggtcaaaatatgggcaaccagaacgggaacccaacaggcactttaggtgcttggggaaacttgctgccacaagagagcacagaaccacaaacgtccacttctcagaatgtgtctttcagcgcacaacctcagaaccttaacactgatggaccaaataacactaaccccatgaactcttcacccaaccctatcaatgcaatgcagacaaatggactgccaaactggggcatggctgttggtatgggggccatcatcccgccccacctgcaaggccttcctggtgctaatggatcatcagtttctcaagtcagtgggggcagtgctgaaggaataagcaattctgtgtggggactgtccccaggtaaccctgccacaggaaatagcaattctgggttcagtcaggggaatggagacactgtgaactcagcattaagtgctaaacaaaatggatccagcagtgctgtgcaaaaggaaggaagtggaggaaatgcttgggattcaggacctcctgctggtcctggaatactcgcctggggaaggggcagtggcaacaatggcgttggtaatatccattcaggagcttggggccaccccagccgaagcacctctaacggtgtgaatggggaatggggaaagcccccaaaccagcattccaacagtgacatcaatgggaaaggatcaacagggtgggagagtcctagtgtcaccagccagaaccctaccgtacagcctggtggtgaacacatgaactcctgggccaaagcggcatcttctggaactacagcaagtgaaggaagtagtgatggttctggcaaccacaatgaaggaagcactgggagggaaggaacgggagaaggccgaaggcgagataaagggattatagaccaagggcacatccagttgccaaggaatgatcttgacccaagagttctgtctaatactggttggggacagactcctgtaaagcaaaacactgcctgggaatttgaagaatcccctaggtctgaaaggaaaaatgacaatgggacagaggcctggggttgtgcagctactcaggcttcaaactcaggggggaagaacgatgggtccatcatgaacagtacaaatacctcttcagtatctgggtgggtcaacgcgccacctgccgctgtgccagcaaacacaggttggggagacagcaacaacaaagcgccaagtggcccgggggtttggggggactcgataagctctactgctgttagtactgctgctgctgccaagagtggccatgcttggagtggggccgcaaatcaggaggacaagtcacccacctggggtgagcctccaaagcccaaatcccaacactggggagatggacaaagatcaaatccagcctggagtgcaggagggggagattgggcagattcatcgtctgtccttggacacttgggggatgggaaaaaaaatggatctggatgggatgctgacagtaataggtcagggtctggttggaatgacaccacgagatctgggaacagtggctggggcaacagcacaaatacaaaggccaatccaggtacaaactggggggagactttaaaacctggcccccaacagaactgggctagcaaaccccaagacaacaatgtgagtaactggggaggagctgcttctgtgaaacagacaggaacagggtggatcggggggccggtaccggtcaaacagaaggacagcagtgaagcaactggctgggaagaaccctctccaccgtccattcgccgcaaaatggaaattgatgatggtacctcagcttggggggacccaagcaactataacaataaaactgtaaacatgtgggatagaaacaacccggtcatccagagcagtaccacgaccaataccaccaccaccaccaccactaccacgagcaacaccacacacagggtcgagacgccgcccccgcaccaggctggtactcagctgaatcgatcaccgttgcttggtccaggtaggaaagtttcatcaggctggggagaaatgcctaatgttcactcaaagactgaaaactcttggggagaaccatcctccccttctaccctggtggataatggcacagcagcatgggggaagccacccagcagtggcagcgggtggggagatcaccctgccgagccgccggtggcatttggaagagctggcgcacctgttgctgcctcagccctgtgcaaaccagcttcaaaatctatgcaagaaggctggggcagtggtggggatgaaatgaacctcagtaccagccagtgggaggatgaagaaggggacgtgtggaataatgctgcttcccaagaaagcacctcctcctgcagctcctgggggaacgcccccaaaaaaggacttcaaaagggcatgaagacgtctggcaagcaggatgaggcctggatcatgagccggctgatcaaacaactcacagacatgggcttcccgagagagccagctgaggaggccttgaagagtaacaatatgaatcttgatcaggccatgagcgctctgctggaaaagaaggtggacgtggacaagcgtgggctgggagtgaccgaccataatggaatggccgccaagcccctcggctgccgcccgccaatctccaaagagtcttccgtggaccgccccacctttcttgacaaggatggcggcctcgtggaagagcccacgccttcaccgttcttgccttccccaagcctgaagctccccctttcacacagtgcactccccagtcaggccctgggtgggattgcctccgggctgggcatgcaaaacttgaattcttctagacagataccgagtggcaatctgggtatgtttggcaatagtggagcagcacaagccaggaccatgcagcagccgccacagccaccagtgcagcctcttaactcttcccagcccagtctccgtgctcaagtgcctcagtttctatcccctcaggttcaagcacagcttttgcagtttgcagcaaaaaacattggtctcaaccctgcactattaacctcgccaattaatcctcaacatatgacgatgttgaaccagctctatcagctgcagctggcataccaacgtttacaaatccagcagcagatgttacaggcccagcgtaatgtgtccggatccatgagacaacaggagcagcaagttgcgcgcacaatcactaatctgcagcagcagatccagcagcaccagcgccagctggcccaggccctgctcgtgaagcagtagaagggtgggcgcgccgacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaa

SEQ ID NO: 19 - DNA, 9398 bp, Homo sapiens
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatctgccaccatggctggaaaggccggtgaaggtgaaatccctgcccctcttgctggtaccgtttctaagatactggtaaaagaaggtgacactgttaaagctggtcaaacagttctggtgctggaggctatgaaaatggagacagaaattaacgctcctactgacggaaaagttgaaaaggtgttagttaaggaaagagatgctgttcaaggtggtcaaggtctaatcaagatcggcgttgctcgaggttatcaaacaagtttgtacaaaaaagcaggctccgcggccgcccccttcaccatggatgctgattctgcctccagttctgaatcagagagaaacatcactatcatggcttcagggaacacaggtggtgaaaaagatggccttcggaatagcactggacttggttcccaaaacaagtttgtagttggtagcagcagcaataatgtgggccatggaagtagtactgggccatggggtttttcccatggagccataataagcacatgtcaggtctctgtggatgctcctgaaagcaaatctgaaagtagcaacaatagaatgaatgcttggggcactgtaagttcttcatcaaatggagggttaaatccaagcactttgaattcagctagcaaccatggtgcctggccagtattagagaacaatggacttgccctaaaagggcctgtagggagtggtagttctggcattaatattcagtgcagtactataggccagatgcctaacaatcagagtattaactctaaagtgagtggtggttctacccatggtacctggggaagccttcaggaaacttgtgaatctgaagtaagtggtacacagaaggtttcattcagtggtcaacctcaaaatattaccactgaaatgactggaccaaataacactactaactttatgacctctagtttaccaaactccggttcagtgcagaataatgagctgcctagtagtaacacaggggcctggcgtgtgagcacaatgaatcatcctcagatgcaggctccatcaggtatgaatggcacttccctttctcaccttagcaatggagagtcaaaaagtggaggctcttatggtactacatggggtgcctatggttctaattactctggagacaaatgttcaggccctaatggccaagctaatggtgacactgtgaatgcaactctaatgcagcctggcgtaaatggtcctatgggcactaactttcaagttaacacaaacaaaggaggtggtgtgtgggaatctggtgcagcaaactcccagagtacatcatggggaagtggaaatggcgcaaattctggaggaagtcgaagaggatggggaacccctgcacaaaacactggcactaatttacccagcgttgagtggaacaaactgcctagcaatcagcattccaatgatagtgcaaatggcaatggtaagacgtttacaaatggatggaaatctactgaggaagaggatcagggttctgccacatctcagacaaatgagcaaagcagtgtgtgggccaaaacaggaggtacagtggagagcgatggtagtacagaaagcactggacgccttgaggaaaaaggaactggggaaagtcagagtagagacagaagaaaaattgatcagcacacattactccaaagcattgtaaacagaactgacttagatccacgtgtcctgtccaactctggttggggacagactcctattaagcagaatactgcctgggatacagaaacatcacctagaggggaacgaaagactgacaatgggacagaggcctggggaagctctgcaacacagacttttaactcaggggcatgtatagataagactagccctaatggtaatgatacctcatctgtatcagggtggggcgatcccaaacctgctctgaggtggggagattccaaaggctcaaactgccagggggggtgggaagatgattctgctgctacaggaatggtcaagagcaatcagtgggggaattgcaaagaggagaaggctgcatggaatgactcgcaaaagaataaacagggatggggtgatggacaaaaatcaagccaagggtggtctgtttctgccagtgataactggggagaaacttcaaggaataaccattggggtgaggccaataagaaatccagctcaggaggtagtgacagtgacaggtccgtttccggttggaacgaacttggtaaaactagttctttcacttggggaaacaacataaatccaaataattcatcaggatgggatgaatcttctaaacctactccttcccagggatggggagaccctccaaagtctaatcagtctctaggttggggagattcgtcaaagccagtcagctctccagactggaacaagcaacaagacattgttggatcttggggaatcccaccagctacaggcaaacctcctggtacaggctggctggggggacctataccagccccagcaaaagaagaagaacccacaggctgggaggaaccatccccagaatctatacgtcgcaaaatggagattgatgatggaacttcagcttggggagatccaagcaaatacaactacaaaaatgtgaacatgtggaacaaaaacgtcccaaatggcaacagccgttcagaccagcaagcacaggtacatcagctgctaacgcctgcaagtgccatctcaaacaaagaggcaagcagtggctctggctggggtgagccctggggggagccttctactccagccacaactgtggataatggtacttcagcatggggtaagcccatagacagtggtcccagctggggggaacccattgctgcggcatccagcacatccacgtggggctccagctctgttggtccacaagcattaagcaaatctgggccaaaatctatgcaagatggctggtgtggtgatgatatgccattgcctggaaatcgccccactggctgggaagaggaagaggatgtggagattggaatgtggaatagtaattcatctcaagagcttaactcatctttaaattggccaccatatacaaagaaaatgtcatcgaagggtctgagtggcaaaaaaaggagaagggaaaggggaatgatgaaaggtggaaacaaacaagaagaagcgtggataaatccatttgttaaacagttttcaaacatcagtttttcgagagactcaccagaggaaaatgtacaaagcaataagatggacctttctggaggaatgttacaagacaaacgaatggagatagataaacatagcctaaatattggtgattacaatcgaacggtcgggaaaggccctggttctcggcctcagatttccaaagagtcttccatggagcgcaatccttattttgataaggatggcattgtagcagatgaatcccaaaacatgcagtttatgtccagtcaaagcatgaagcttcccccttcaaatagtgcactacctaaccaggcccttggctccatagcagggctgggtatgcaaaacttgaattctgttagacagaatggcaatcccagtatgtttggtgttggaaacacagcagcacaaccccggggcatgcagcagcctccagcacaacctcttagttcatctcagcctaatctccgtgctcaagtgcctcctccattactctcccctcaggttccagtttcattgctgaagtatgcaccaaacaacggtggcctgaatccactctttggccctcaacaggtagccatgctgaaccagctatcccagctaaaccagctttctcagatctcccagttacagcgattgttagcgcagcagcaaagggcgcagagtcagagaagcgtgccttctgggaaccggccgcagcaagaccagcagggtcgacctcttagtgtgcagcagcaaatgatgcaacaatctcgtcaacttgatccaaacctgttggtgtagaagggtgggcgcgccgacccagctttcttgtacaaagtggttcgataacgaattctgcagatatccagcacagtggcggccgctcgagtctagagggcccgtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 20 - DNA, 9428 bp, Homo sapiens
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatctgccaccatggctggaaaggccggtgaaggtgaaatccctgcccctcttgctggtaccgtttctaagatactggtaaaagaaggtgacactgttaaagctggtcaaacagttctggtgctggaggctatgaaaatggagacagaaattaacgctcctactgacggaaaagttgaaaaggtgttagttaaggaaagagatgctgttcaaggtggtcaaggtctaatcaagatcggcgttgctcgaggttatcaaacaagtttgtacaaaaaagcaggctccgcggccgcccccttcaccatgagagagaaggagcaagaaagggaagaacagttaatggaagacaagaaaaggaagaaagaggataaaaagaaaaaagaagccactcagaaggtcacggaacaaaaaaccaaagtgcccgaagtgacgaaaccaagtttaagccaaccaacggccgccagcccaattggcagctctccatcgccaccagtcaatggtggcaacaatgccaaaagggtggcagtgccgaacggacaaccgccaagcgccgcccgctacatgcctcgggaggtgccgccgcgattccgttgccagcaggaccacaaagtgttactaaaacgtgggcagccccctccaccgtcctgcatgctccttgggggtggggcagggcctcctccctgcacagcacctggagcaaacccaaacaacgcacaagtgacaggagcgctgctgcagagtgagagtgggactgcgccagactcaacccttggaggtgctgctgcttcaaattatgcaaattccacttggggctcgggagcctcctccaacaacggcacctcccccaacccaattcacatctgggacaaggtgattgtagacgggtctgacatggaagagtggccttgtattgccagcaaagacactgaatcttcttccgaaaacaccaccgataacaacagtgcctcgaaccctggctctgagaagagcactctgccaggaagcaccactagtaacaaaggaaaagggagccagtgccagtctgcaagttctgggaacgaatgtaatcttggggtctggaaatctgaccctaaggctaaatctgttcaatcttccaactctactacagagaacaacaatggactaggaaattggaggaatgtgagtggtcaggatagaattggacctggctctggcttcagcaactttaacccaaatagcaacccatctgcctggccagcactggtccaagaaggaacttctaggaaaggggcattggaaacagataatagtaattccagtgcacaggttagcacagtaggtcagacatccagggaacagcagtcaaagatggaaaatgcgggtgttaattttgttgtctctggcagagaacaggctcaaattcataacactgatggaccaaaaaatggaaacactaactccttgaacttaagttcaccaaaccccatggagaataagggaatgccctttggaatgggcttggggaacacctccaggagcactgatgccccttcacaaagcactggagatcgaaagactgggagtgttggatcttggggtgcagctagggggccttctggaactgacacagtctctggacaaagcaattctggaaacaatgggaacaatggaaaagagagagaggactcctggaaaggagcttctgttcagaaatcaactgggtcaaaaaatgactcttgggacaacaataacaggtctacgggtgggtcctggaactttggcccccaggactctaatgacaacaaatggggtgaagggaacaaaatgacatctggggtctctcagggagaatggaaacagccgactgggtctgatgagttgaaaattggagaatggagtggtccaaaccaaccaaattctagcactggagcatgggacaatcaaaagggccaccccctccctgaaaaccaaggcaatgcccaggctccctgttggggaagatcttccagctccacaggaagtgaagttggaggtcaaagcactggaagcaaccacaaagcaggaagtagtgacagtcataactctggccgtcggtcgtacaggcccacacatcctgattgtcaggctgtcttgcagactcttttgagccgaactgatttggaccccagggtgctctcaaacactggctggggccaaactcaaattaagcaggacacagtgtgggacattgaagaggtgccaaggcctgaggggaaatctgacaaaggaactgaggggtgggagagcgctgccacacagaccaagaactcagggggctggggagatgcacccagccaaagcaatcaaatgaagtctggatggggggagctctcagcctctacagagtggaaagaccccaagaacacaggaggctggaatgactacaagaacaacaactcttccaactggggaggaggacgacctgatgaaaagacaccttcctcttggaatgagaatcccagcaaggatcaggggtggggaggtggacgccagcccaatcaaggatggtcttctggaaagaatggttggggggaggaagtcgatcagacaaaaaacagcaattgggaaagttctgcaagtaaacctgtgtctgggtggggtgaaggagggcagaatgaaatcgggacttggggtaatggtggcaatgcaagcctagcttcaaaaggtgggtgggaggattgcaaaagatccccagcatggaatgagacgggccgacagcccaattcctggaataaacaacaccaacagcagcagcccccacagcagccgccgccaccacaaccagaggcttctggttcgtggggaggcccacccccaccacctccaggcaacgttcgaccttccaattccagctggagcagcgggccacagcctgcaacacctaaggatgaggaacccagtggttgggaagagccatccccacagtcaattagtcggaaaatggacattgatgatggcacttcagcatggggagaccctaacagttataactacaagaatgtgaatctgtgggataagaattcccaagggggcccagcacctcgagaaccaaacctgcccaccccaatgaccagtaaatcggcatcagattccaaatctatgcaagacggctggggggagagtgacgggccagtcacaggagctcgccatcccagctgggaagaggaggaggatggaggagtctggaacaccactggctctcagggcagtgcttcctcccacaactcagcaagctggggacaaggaggaaagaaacaaatgaagtgctcactcaaaggaggaaacaatgattcatggatgaatcctcttgccaaacagttttcaaatatgggattgctgagtcagactgaagataatccaagcagcaaaatggatttgtctgtaggaagcctttcagataaaaaatttgatgtggacaagcgagcgatgaatctcggggattttaatgatatcatgaggaaggatcgatctgggttccgtccacctaattccaaagacatgggaaccacagatagtgggccttattttgagaagggcggtagtcatggtttgtttggaaacagcacagcacaatcgagaggtctgcacacacccgtgcagccactaaattcttctcccagtctccgggcgcaagtgcctccccagtttatttccccccaggtttctgcctcaatgctcaagcagtttcccaacagtggcctgagtccaggtcttttcaatgtggggccccagttatctcctcaacaaattgccatgctgagccagcttccacaaattccccagtttcagttggcatgtcagcttctcttgcagcagcagcaacagcagcagttgttacagaaccagagaaagatttctcaagctgtacgccaacagcaagagcagcagctggctcgaatggtgagtgcactgcagcagcagcagcagcagcagcagaggcagccaggcatgtagaagggtgggcgcgccgacccagctttcttgtacaaagtggttcgataacgaattctgcagatatccagcacagtggcggccgctcgagtctagagggcccgtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 21 - DNA, 9401 bp, Homo sapiens
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatctgccaccatggctggaaaggccggtgaaggtgaaatccctgcccctcttgctggtaccgtttctaagatactggtaaaagaaggtgacactgttaaagctggtcaaacagttctggtgctggaggctatgaaaatggagacagaaattaacgctcctactgacggaaaagttgaaaaggtgttagttaaggaaagagatgctgttcaaggtggtcaaggtctaatcaagatcggcgttgctcgaggttatcaaacaagtttgtacaaaaaagcaggctccgcggccgcccccttcaccatggctacagggagtgcccagggcaacttcactggacataccaagaagacaaatggcaataatggcaccaatggcgcactcgtccaaagcccttctaatcagagtgcccttggagcagggggagcgaacagtaatggaagtgcggccagagtgtggggtgtagccacaggctccagctctggcctggctcactgctctgtcagtggtggggatggaaaaatggacactatgattggagatgggagaagtcagaattgctggggtgcttccaactccaatgctggcattaatcttaaccttaatcctaatgccaacccagctgcctggcctgtacttggacatgaaggaaccgtggcgacaggcaacccttccagtatttgcagtccagtcagtgccataggtcaaaatatgggcaaccagaacgggaacccaacaggcactttaggtgcttggggaaacttgctgccacaagagagcacagaaccacaaacgtccacttctcagaatgtgtctttcagcgcacaacctcagaaccttaacactgatggaccaaataacactaaccccatgaactcttcacccaaccctatcaatgcaatgcagacaaatggactgccaaactggggcatggctgttggtatgggggccatcatcccgccccacctgcaaggccttcctggtgctaatggatcatcagtttctcaagtcagtgggggcagtgctgaaggaataagcaattctgtgtggggactgtccccaggtaaccctgccacaggaaatagcaattctgggttcagtcaggggaatggagacactgtgaactcagcattaagtgctaaacaaaatggatccagcagtgctgtgcaaaaggaaggaagtggaggaaatgcttgggattcaggacctcctgctggtcctggaatactcgcctggggaaggggcagtggcaacaatggcgttggtaatatccattcaggagcttggggccaccccagccgaagcacctctaacggtgtgaatggggaatggggaaagcccccaaaccagcattccaacagtgacatcaatgggaaaggatcaacagggtgggagagtcctagtgtcaccagccagaaccctaccgtacagcctggtggtgaacacatgaactcctgggccaaagcggcatcttctggaactacagcaagtgaaggaagtagtgatggttctggcaaccacaatgaaggaagcactgggagggaaggaacgggagaaggccgaaggcgagataaagggattatagaccaagggcacatccagttgccaaggaatgatcttgacccaagagttctgtctaatactggttggggacagactcctgtaaagcaaaacactgcctgggaatttgaagaatcccctaggtctgaaaggaaaaatgacaatgggacagaggcctggggttgtgcagctactcaggcttcaaactcaggggggaagaacgatgggtccatcatgaacagtacaaatacctcttcagtatctgggtgggtcaacgcgccacctgccgctgtgccagcaaacacaggttggggagacagcaacaacaaagcgccaagtggcccgggggtttggggggactcgataagctctactgctgttagtactgctgctgctgccaagagtggccatgcttggagtggggccgcaaatcaggaggacaagtcacccacctggggtgagcctccaaagcccaaatcccaacactggggagatggacaaagatcaaatccagcctggagtgcaggagggggagattgggcagattcatcgtctgtccttggacacttgggggatgggaaaaaaaatggatctggatgggatgctgacagtaataggtcagggtctggttggaatgacaccacgagatctgggaacagtggctggggcaacagcacaaatacaaaggccaatccaggtacaaactggggggagactttaaaacctggcccccaacagaactgggctagcaaaccccaagacaacaatgtgagtaactggggaggagctgcttctgtgaaacagacaggaacagggtggatcggggggccggtaccggtcaaacagaaggacagcagtgaagcaactggctgggaagaaccctctccaccgtccattcgccgcaaaatggaaattgatgatggtacctcagcttggggggacccaagcaactataacaataaaactgtaaacatgtgggatagaaacaacccggtcatccagagcagtaccacgaccaataccaccaccaccaccaccactaccacgagcaacaccacacacagggtcgagacgccgcccccgcaccaggctggtactcagctgaatcgatcaccgttgcttggtccaggtaggaaagtttcatcaggctggggagaaatgcctaatgttcactcaaagactgaaaactcttggggagaaccatcctccccttctaccctggtggataatggcacagcagcatgggggaagccacccagcagtggcagcgggtggggagatcaccctgccgagccgccggtggcatttggaagagctggcgcacctgttgctgcctcagccctgtgcaaaccagcttcaaaatctatgcaagaaggctggggcagtggtggggatgaaatgaacctcagtaccagccagtgggaggatgaagaaggggacgtgtggaataatgctgcttcccaagaaagcacctcctcctgcagctcctgggggaacgcccccaaaaaaggacttcaaaagggcatgaagacgtctggcaagcaggatgaggcctggatcatgagccggctgatcaaacaactcacagacatgggcttcccgagagagccagctgaggaggccttgaagagtaacaatatgaatcttgatcaggccatgagcgctctgctggaaaagaaggtggacgtggacaagcgtgggctgggagtgaccgaccataatggaatggccgccaagcccctcggctgccgcccgccaatctccaaagagtcttccgtggaccgccccacctttcttgacaaggatggcggcctcgtggaagagcccacgccttcaccgttcttgccttccccaagcctgaagctccccctttcacacagtgcactccccagtcaggccctgggtgggattgcctccgggctgggcatgcaaaacttgaattcttctagacagataccgagtggcaatctgggtatgtttggcaatagtggagcagcacaagccaggaccatgcagcagccgccacagccaccagtgcagcctcttaactcttcccagcccagtctccgtgctcaagtgcctcagtttctatcccctcaggttcaagcacagcttttgcagtttgcagcaaaaaacattggtctcaaccctgcactattaacctcgccaattaatcctcaacatatgacgatgttgaaccagctctatcagctgcagctggcataccaacgtttacaaatccagcagcagatgttacaggcccagcgtaatgtgtccggatccatgagacaacaggagcagcaagttgcgcgcacaatcactaatctgcagcagcagatccagcagcaccagcgccagctggcccaggccctgctcgtgaagcagtagaagggtgggcgcgccgacccagctttcttgtacaaagtggttcgataacgaattctgcagatatccagcacagtggcggccgctcgagtctagagggcccgtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 22 - DNA, 9046 bp, Homo sapiens
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggcggccgcagatctctcgaggttgatctgccaccatggactacaaggatgacgatgacaagctcgatggaggatacccatacgatgttccagattacgctgctcgaggttatcaaacaagtttgtacaaaaaagcaggctccgcggccgcccccttcaccatggatgctgattctgcctccagttctgaatcagagagaaacatcactatcatggcttcagggaacacaggtggtgaaaaagatggccttcggaatagcactggacttggttcccaaaacaagtttgtagttggtagcagcagcaataatgtgggccatggaagtagtactgggccatggggtttttcccatggagccataataagcacatgtcaggtctctgtggatgctcctgaaagcaaatctgaaagtagcaacaatagaatgaatgcttggggcactgtaagttcttcatcaaatggagggttaaatccaagcactttgaattcagctagcaaccatggtgcctggccagtattagagaacaatggacttgccctaaaagggcctgtagggagtggtagttctggcattaatattcagtgcagtactataggccagatgcctaacaatcagagtattaactctaaagtgagtggtggttctacccatggtacctggggaagccttcaggaaacttgtgaatctgaagtaagtggtacacagaaggtttcattcagtggtcaacctcaaaatattaccactgaaatgactggaccaaataacactactaactttatgacctctagtttaccaaactccggttcagtgcagaataatgagctgcctagtagtaacacaggggcctggcgtgtgagcacaatgaatcatcctcagatgcaggctccatcaggtatgaatggcacttccctttctcaccttagcaatggagagtcaaaaagtggaggctcttatggtactacatggggtgcctatggttctaattactctggagacaaatgttcaggccctaatggccaagctaatggtgacactgtgaatgcaactctaatgcagcctggcgtaaatggtcctatgggcactaactttcaagttaacacaaacaaaggaggtggtgtgtgggaatctggtgcagcaaactcccagagtacatcatggggaagtggaaatggcgcaaattctggaggaagtcgaagaggatggggaacccctgcacaaaacactggcactaatttacccagcgttgagtggaacaaactgcctagcaatcagcattccaatgatagtgcaaatggcaatggtaagacgtttacaaatggatggaaatctactgaggaagaggatcagggttctgccacatctcagacaaatgagcaaagcagtgtgtgggccaaaacaggaggtacagtggagagcgatggtagtacagaaagcactggacgccttgaggaaaaaggaactggggaaagtcagagtagagacagaagaaaaattgatcagcacacattactccaaagcattgtaaacagaactgacttagatccacgtgtcctgtccaactctggttggggacagactcctattaagcagaatactgcctgggatacagaaacatcacctagaggggaacgaaagactgacaatgggacagaggcctggggaagctctgcaacacagacttttaactcaggggcatgtatagataagactagccctaatggtaatgatacctcatctgtatcagggtggggcgatcccaaacctgctctgaggtggggagattccaaaggctcaaactgccagggggggtgggaagatgattctgctgctacaggaatggtcaagagcaatcagtgggggaattgcaaagaggagaaggctgcatggaatgactcgcaaaagaataaacagggatggggtgatggacaaaaatcaagccaagggtggtctgtttctgccagtgataactggggagaaacttcaaggaataaccattggggtgaggccaataagaaatccagctcaggaggtagtgacagtgacaggtccgtttccggttggaacgaacttggtaaaactagttctttcacttggggaaacaacataaatccaaataattcatcaggatgggatgaatcttctaaacctactccttcccagggatggggagaccctccaaagtctaatcagtctctaggttggggagattcgtcaaagccagtcagctctccagactggaacaagcaacaagacattgttggatcttggggaatcccaccagctacaggcaaacctcctggtacaggctggctggggggacctataccagccccagcaaaagaagaagaacccacaggctgggaggaaccatccccagaatctatacgtcgcaaaatggagattgatgatggaacttcagcttggggagatccaagcaaatacaactacaaaaatgtgaacatgtggaacaaaaacgtcccaaatggcaacagccgttcagaccagcaagcacaggtacatcagctgctaacgcctgcaagtgccatctcaaacaaagaggcaagcagtggctctggctggggtgagccctggggggagccttctactccagccacaactgtggataatggtacttcagcatggggtaagcccatagacagtggtcccagctggggggaacccattgctgcggcatccagcacatccacgtggggctccagctctgttggtccacaagcattaagcaaatctgggccaaaatctatgcaagatggctggtgtggtgatgatatgccattgcctggaaatcgccccactggctgggaagaggaagaggatgtggagattggaatgtggaatagtaattcatctcaagagcttaactcatctttaaattggccaccatatacaaagaaaatgtcatcgaagggtctgagtggcaaaaaaaggagaagggaaaggggaatgatgaaaggtggaaacaaacaagaagaagcgtggataaatccatttgttaaacagttttcaaacatcagtttttcgagagactcaccagaggaaaatgtacaaagcaataagatggacctttctggaggaatgttacaagacaaacgaatggagatagataaacatagcctaaatattggtgattacaatcgaacggtcgggaaaggccctggttctcggcctcagatttccaaagagtcttccatggagcgcaatccttattttgataaggatggcattgtagcagatgaatcccaaaacatgcagtttatgtccagtcaaagcatgaagcttcccccttcaaatagtgcactacctaaccaggcccttggctccatagcagggctgggtatgcaaaacttgaattctgttagacagaatggcaatcccagtatgtttggtgttggaaacacagcagcacaaccccggggcatgcagcagcctccagcacaacctcttagttcatctcagcctaatctccgtgctcaagtgcctcctccattactctcccctcaggttccagtttcattgctgaagtatgcaccaaacaacggtggcctgaatccactctttggccctcaacaggtagccatgctgaaccagctatcccagctaaaccagctttctcagatctcccagttacagcgattgttagcgcagcagcaaagggcgcagagtcagagaagcgtgccttctgggaaccggccgcagcaagaccagcagggtcgacctcttagtgtgcagcagcaaatgatgcaacaatctcgtcaacttgatccaaacctgttggtgtagaagggtgggcgcgccgacccagctttcttgtacaaagtggttcgataacgaattccgccccccccccctaacgttactggccgaagccgcttggaataaggccggtgtgcgtttgtctatatgttattttccaccatattgccgtcttttggcaatgtgagggccggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 23 - DNA, 7529 bp, Homo sapiens
gagttcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactagaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgggcccaccatggcatcaatgcagaagctgatctcagaggaggacctgcttatggccatggaggcccgaattatcacaagtttgtacaaaaaagcaggctccgcggccgcccccttcaccatggatgctgattctgcctccagttctgaatcagagagaaacatcactatcatggcttcagggaacacaggtggtgaaaaagatggccttcggaatagcactggacttggttcccaaaacaagtttgtagttggtagcagcagcaataatgtgggccatggaagtagtactgggccatggggtttttcccatggagccataataagcacatgtcaggtctctgtggatgctcctgaaagcaaatctgaaagtagcaacaatagaatgaatgcttggggcactgtaagttcttcatcaaatggagggttaaatccaagcactttgaattcagctagcaaccatggtgcctggccagtattagagaacaatggacttgccctaaaagggcctgtagggagtggtagttctggcattaatattcagtgcagtactataggccagatgcctaacaatcagagtattaactctaaagtgagtggtggttctacccatggtacctggggaagccttcaggaaacttgtgaatctgaagtaagtggtacacagaaggtttcattcagtggtcaacctcaaaatattaccactgaaatgactggaccaaataacactactaactttatgacctctagtttaccaaactccggttcagtgcagaataatgagctgcctagtagtaacacaggggcctggcgtgtgagcacaatgaatcatcctcagatgcaggctccatcaggtatgaatggcacttccctttctcaccttagcaatggagagtcaaaaagtggaggctcttatggtactacatggggtgcctatggttctaattactctggagacaaatgttcaggccctaatggccaagctaatggtgacactgtgaatgcaactctaatgcagcctggcgtaaatggtcctatgggcactaactttcaagttaacacaaacaaaggaggtggtgtgtgggaatctggtgcagcaaactcccagagtacatcatggggaagtggaaatggcgcaaattctggaggaagtcgaagaggatggggaacccctgcacaaaacactggcactaatttacccagcgttgagtggaacaaactgcctagcaatcagcattccaatgatagtgcaaatggcaatggtaagacgtttacaaatggatggaaatctactgaggaagaggatcagggttctgccacatctcagacaaatgagcaaagcagtgtgtgggccaaaacaggaggtacagtggagagcgatggtagtacagaaagcactggacgccttgaggaaaaaggaactggggaaagtcagagtagagacagaagaaaaattgatcagcacacattactccaaagcattgtaaacagaactgacttagatccacgtgtcctgtccaactctggttggggacagactcctattaagcagaatactgcctgggatacagaaacatcacctagaggggaacgaaagactgacaatgggacagaggcctggggaagctctgcaacacagacttttaactcaggggcatgtatagataagactagccctaatggtaatgatacctcatctgtatcagggtggggcgatcccaaacctgctctgaggtggggagattccaaaggctcaaactgccagggggggtgggaagatgattctgctgctacaggaatggtcaagagcaatcagtgggggaattgcaaagaggagaaggctgcatggaatgactcgcaaaagaataaacagggatggggtgatggacaaaaatcaagccaagggtggtctgtttctgccagtgataactggggagaaacttcaaggaataaccattggggtgaggccaataagaaatccagctcaggaggtagtgacagtgacaggtccgtttccggttggaacgaacttggtaaaactagttctttcacttggggaaacaacataaatccaaataattcatcaggatgggatgaatcttctaaacctactccttcccagggatggggagaccctccaaagtctaatcagtctctaggttggggagattcgtcaaagccagtcagctctccagactggaacaagcaacaagacattgttggatcttggggaatcccaccagctacaggcaaacctcctggtacaggctggctggggggacctataccagccccagcaaaagaagaagaacccacaggctgggaggaaccatccccagaatctatacgtcgcaaaatggagattgatgatggaacttcagcttggggagatccaagcaaatacaactacaaaaatgtgaacatgtggaacaaaaacgtcccaaatggcaacagccgttcagaccagcaagcacaggtacatcagctgctaacgcctgcaagtgccatctcaaacaaagaggcaagcagtggctctggctggggtgagccctggggggagccttctactccagccacaactgtggataatggtacttcagcatggggtaagcccatagacagtggtcccagctggggggaacccattgctgcggcatccagcacatccacgtggggctccagctctgttggtccacaagcattaagcaaatctgggccaaaatctatgcaagatggctggtgtggtgatgatatgccattgcctggaaatcgccccactggctgggaagaggaagaggatgtggagattggaatgtggaatagtaattcatctcaagagcttaactcatctttaaattggccaccatatacaaagaaaatgtcatcgaagggtctgagtggcaaaaaaaggagaagggaaaggggaatgatgaaaggtggaaacaaacaagaagaagcgtggataaatccatttgttaaacagttttcaaacatcagtttttcgagagactcaccagaggaaaatgtacaaagcaataagatggacctttctggaggaatgttacaagacaaacgaatggagatagataaacatagcctaaatattggtgattacaatcgaacggtcgggaaaggccctggttctcggcctcagatttccaaagagtcttccatggagcgcaatccttattttgataaggatggcattgtagcagatgaatcccaaaacatgcagtttatgtccagtcaaagcatgaagcttcccccttcaaatagtgcactacctaaccaggcccttggctccatagcagggctgggtatgcaaaacttgaattctgttagacagaatggcaatcccagtatgtttggtgttggaaacacagcagcacaaccccggggcatgcagcagcctccagcacaacctcttagttcatctcagcctaatctccgtgctcaagtgcctcctccattactctcccctcaggttccagtttcattgctgaagtatgcaccaaacaacggtggcctgaatccactctttggccctcaacaggtagccatgctgaaccagctatcccagctaaaccagctttctcagatctcccagttacagcgattgttagcgcagcagcaaagggcgcagagtcagagaagcgtgccttctgggaaccggccgcagcaagaccagcagggtcgacctcttagtgtgcagcagcaaatgatgcaacaatctcgtcaacttgatccaaacctgttggtgtagaagggtgggcgcgccgacccagctttcttgtacaaagtggtgataattcggtcgaccgagatctctcgaggtaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgatctgcaggcatgctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 24 - DNA, 7559 bp, Homo sapiens
gagttcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactagaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgggcccaccatggcatcaatgcagaagctgatctcagaggaggacctgcttatggccatggaggcccgaattatcacaagtttgtacaaaaaagcaggctccgcggccgcccccttcaccatgagagagaaggagcaagaaagggaagaacagttaatggaagacaagaaaaggaagaaagaggataaaaagaaaaaagaagccactcagaaggtcacggaacaaaaaaccaaagtgcccgaagtgacgaaaccaagtttaagccaaccaacggccgccagcccaattggcagctctccatcgccaccagtcaatggtggcaacaatgccaaaagggtggcagtgccgaacggacaaccgccaagcgccgcccgctacatgcctcgggaggtgccgccgcgattccgttgccagcaggaccacaaagtgttactaaaacgtgggcagccccctccaccgtcctgcatgctccttgggggtggggcagggcctcctccctgcacagcacctggagcaaacccaaacaacgcacaagtgacaggagcgctgctgcagagtgagagtgggactgcgccagactcaacccttggaggtgctgctgcttcaaattatgcaaattccacttggggctcgggagcctcctccaacaacggcacctcccccaacccaattcacatctgggacaaggtgattgtagacgggtctgacatggaagagtggccttgtattgccagcaaagacactgaatcttcttccgaaaacaccaccgataacaacagtgcctcgaaccctggctctgagaagagcactctgccaggaagcaccactagtaacaaaggaaaagggagccagtgccagtctgcaagttctgggaacgaatgtaatcttggggtctggaaatctgaccctaaggctaaatctgttcaatcttccaactctactacagagaacaacaatggactaggaaattggaggaatgtgagtggtcaggatagaattggacctggctctggcttcagcaactttaacccaaatagcaacccatctgcctggccagcactggtccaagaaggaacttctaggaaaggggcattggaaacagataatagtaattccagtgcacaggttagcacagtaggtcagacatccagggaacagcagtcaaagatggaaaatgcgggtgttaattttgttgtctctggcagagaacaggctcaaattcataacactgatggaccaaaaaatggaaacactaactccttgaacttaagttcaccaaaccccatggagaataagggaatgccctttggaatgggcttggggaacacctccaggagcactgatgccccttcacaaagcactggagatcgaaagactgggagtgttggatcttggggtgcagctagggggccttctggaactgacacagtctctggacaaagcaattctggaaacaatgggaacaatggaaaagagagagaggactcctggaaaggagcttctgttcagaaatcaactgggtcaaaaaatgactcttgggacaacaataacaggtctacgggtgggtcctggaactttggcccccaggactctaatgacaacaaatggggtgaagggaacaaaatgacatctggggtctctcagggagaatggaaacagccgactgggtctgatgagttgaaaattggagaatggagtggtccaaaccaaccaaattctagcactggagcatgggacaatcaaaagggccaccccctccctgaaaaccaaggcaatgcccaggctccctgttggggaagatcttccagctccacaggaagtgaagttggaggtcaaagcactggaagcaaccacaaagcaggaagtagtgacagtcataactctggccgtcggtcgtacaggcccacacatcctgattgtcaggctgtcttgcagactcttttgagccgaactgatttggaccccagggtgctctcaaacactggctggggccaaactcaaattaagcaggacacagtgtgggacattgaagaggtgccaaggcctgaggggaaatctgacaaaggaactgaggggtgggagagcgctgccacacagaccaagaactcagggggctggggagatgcacccagccaaagcaatcaaatgaagtctggatggggggagctctcagcctctacagagtggaaagaccccaagaacacaggaggctggaatgactacaagaacaacaactcttccaactggggaggaggacgacctgatgaaaagacaccttcctcttggaatgagaatcccagcaaggatcaggggtggggaggtggacgccagcccaatcaaggatggtcttctggaaagaatggttggggggaggaagtcgatcagacaaaaaacagcaattgggaaagttctgcaagtaaacctgtgtctgggtggggtgaaggagggcagaatgaaatcgggacttggggtaatggtggcaatgcaagcctagcttcaaaaggtgggtgggaggattgcaaaagatccccagcatggaatgagacgggccgacagcccaattcctggaataaacaacaccaacagcagcagcccccacagcagccgccgccaccacaaccagaggcttctggttcgtggggaggcccacccccaccacctccaggcaacgttcgaccttccaattccagctggagcagcgggccacagcctgcaacacctaaggatgaggaacccagtggttgggaagagccatccccacagtcaattagtcggaaaatggacattgatgatggcacttcagcatggggagaccctaacagttataactacaagaatgtgaatctgtgggataagaattcccaagggggcccagcacctcgagaaccaaacctgcccaccccaatgaccagtaaatcggcatcagattccaaatctatgcaagacggctggggggagagtgacgggccagtcacaggagctcgccatcccagctgggaagaggaggaggatggaggagtctggaacaccactggctctcagggcagtgcttcctcccacaactcagcaagctggggacaaggaggaaagaaacaaatgaagtgctcactcaaaggaggaaacaatgattcatggatgaatcctcttgccaaacagttttcaaatatgggattgctgagtcagactgaagataatccaagcagcaaaatggatttgtctgtaggaagcctttcagataaaaaatttgatgtggacaagcgagcgatgaatctcggggattttaatgatatcatgaggaaggatcgatctgggttccgtccacctaattccaaagacatgggaaccacagatagtgggccttattttgagaagggcggtagtcatggtttgtttggaaacagcacagcacaatcgagaggtctgcacacacccgtgcagccactaaattcttctcccagtctccgggcgcaagtgcctccccagtttatttccccccaggtttctgcctcaatgctcaagcagtttcccaacagtggcctgagtccaggtcttttcaatgtggggccccagttatctcctcaacaaattgccatgctgagccagcttccacaaattccccagtttcagttggcatgtcagcttctcttgcagcagcagcaacagcagcagttgttacagaaccagagaaagatttctcaagctgtacgccaacagcaagagcagcagctggctcgaatggtgagtgcactgcagcagcagcagcagcagcagcagaggcagccaggcatgtagaagggtgggcgcgccgacccagctttcttgtacaaagtggtgataattcggtcgaccgagatctctcgaggtaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgatctgcaggcatgctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 25 - DNA, 7532 bp, Homo sapiens
gagttcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactagaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgggcccaccatggcatcaatgcagaagctgatctcagaggaggacctgcttatggccatggaggcccgaattatcacaagtttgtacaaaaaagcaggctccgcggccgcccccttcaccatggctacagggagtgcccagggcaacttcactggacataccaagaagacaaatggcaataatggcaccaatggcgcactcgtccaaagcccttctaatcagagtgcccttggagcagggggagcgaacagtaatggaagtgcggccagagtgtggggtgtagccacaggctccagctctggcctggctcactgctctgtcagtggtggggatggaaaaatggacactatgattggagatgggagaagtcagaattgctggggtgcttccaactccaatgctggcattaatcttaaccttaatcctaatgccaacccagctgcctggcctgtacttggacatgaaggaaccgtggcgacaggcaacccttccagtatttgcagtccagtcagtgccataggtcaaaatatgggcaaccagaacgggaacccaacaggcactttaggtgcttggggaaacttgctgccacaagagagcacagaaccacaaacgtccacttctcagaatgtgtctttcagcgcacaacctcagaaccttaacactgatggaccaaataacactaaccccatgaactcttcacccaaccctatcaatgcaatgcagacaaatggactgccaaactggggcatggctgttggtatgggggccatcatcccgccccacctgcaaggccttcctggtgctaatggatcatcagtttctcaagtcagtgggggcagtgctgaaggaataagcaattctgtgtggggactgtccccaggtaaccctgccacaggaaatagcaattctgggttcagtcaggggaatggagacactgtgaactcagcattaagtgctaaacaaaatggatccagcagtgctgtgcaaaaggaaggaagtggaggaaatgcttgggattcaggacctcctgctggtcctggaatactcgcctggggaaggggcagtggcaacaatggcgttggtaatatccattcaggagcttggggccaccccagccgaagcacctctaacggtgtgaatggggaatggggaaagcccccaaaccagcattccaacagtgacatcaatgggaaaggatcaacagggtgggagagtcctagtgtcaccagccagaaccctaccgtacagcctggtggtgaacacatgaactcctgggccaaagcggcatcttctggaactacagcaagtgaaggaagtagtgatggttctggcaaccacaatgaaggaagcactgggagggaaggaacgggagaaggccgaaggcgagataaagggattatagaccaagggcacatccagttgccaaggaatgatcttgacccaagagttctgtctaatactggttggggacagactcctgtaaagcaaaacactgcctgggaatttgaagaatcccctaggtctgaaaggaaaaatgacaatgggacagaggcctggggttgtgcagctactcaggcttcaaactcaggggggaagaacgatgggtccatcatgaacagtacaaatacctcttcagtatctgggtgggtcaacgcgccacctgccgctgtgccagcaaacacaggttggggagacagcaacaacaaagcgccaagtggcccgggggtttggggggactcgataagctctactgctgttagtactgctgctgctgccaagagtggccatgcttggagtggggccgcaaatcaggaggacaagtcacccacctggggtgagcctccaaagcccaaatcccaacactggggagatggacaaagatcaaatccagcctggagtgcaggagggggagattgggcagattcatcgtctgtccttggacacttgggggatgggaaaaaaaatggatctggatgggatgctgacagtaataggtcagggtctggttggaatgacaccacgagatctgggaacagtggctggggcaacagcacaaatacaaaggccaatccaggtacaaactggggggagactttaaaacctggcccccaacagaactgggctagcaaaccccaagacaacaatgtgagtaactggggaggagctgcttctgtgaaacagacaggaacagggtggatcggggggccggtaccggtcaaacagaaggacagcagtgaagcaactggctgggaagaaccctctccaccgtccattcgccgcaaaatggaaattgatgatggtacctcagcttggggggacccaagcaactataacaataaaactgtaaacatgtgggatagaaacaacccggtcatccagagcagtaccacgaccaataccaccaccaccaccaccactaccacgagcaacaccacacacagggtcgagacgccgcccccgcaccaggctggtactcagctgaatcgatcaccgttgcttggtccaggtaggaaagtttcatcaggctggggagaaatgcctaatgttcactcaaagactgaaaactcttggggagaaccatcctccccttctaccctggtggataatggcacagcagcatgggggaagccacccagcagtggcagcgggtggggagatcaccctgccgagccgccggtggcatttggaagagctggcgcacctgttgctgcctcagccctgtgcaaaccagcttcaaaatctatgcaagaaggctggggcagtggtggggatgaaatgaacctcagtaccagccagtgggaggatgaagaaggggacgtgtggaataatgctgcttcccaagaaagcacctcctcctgcagctcctgggggaacgcccccaaaaaaggacttcaaaagggcatgaagacgtctggcaagcaggatgaggcctggatcatgagccggctgatcaaacaactcacagacatgggcttcccgagagagccagctgaggaggccttgaagagtaacaatatgaatcttgatcaggccatgagcgctctgctggaaaagaaggtggacgtggacaagcgtgggctgggagtgaccgaccataatggaatggccgccaagcccctcggctgccgcccgccaatctccaaagagtcttccgtggaccgccccacctttcttgacaaggatggcggcctcgtggaagagcccacgccttcaccgttcttgccttccccaagcctgaagctccccctttcacacagtgcactccccagtcaggccctgggtgggattgcctccgggctgggcatgcaaaacttgaattcttctagacagataccgagtggcaatctgggtatgtttggcaatagtggagcagcacaagccaggaccatgcagcagccgccacagccaccagtgcagcctcttaactcttcccagcccagtctccgtgctcaagtgcctcagtttctatcccctcaggttcaagcacagcttttgcagtttgcagcaaaaaacattggtctcaaccctgcactattaacctcgccaattaatcctcaacatatgacgatgttgaaccagctctatcagctgcagctggcataccaacgtttacaaatccagcagcagatgttacaggcccagcgtaatgtgtccggatccatgagacaacaggagcagcaagttgcgcgcacaatcactaatctgcagcagcagatccagcagcaccagcgccagctggcccaggccctgctcgtgaagcagtagaagggtgggcgcgccgacccagctttcttgtacaaagtggtgataattcggtcgaccgagatctctcgaggtaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgatctgcaggcatgctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 26 - DNA, 11468 bp, Artificial Sequence
  Lentiviral vector
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggcggccgcagatctctcgaggttgatctgccaccatggactacaaggatgacgatgacaagctcgatggaggatacccatacgatgttccagattacgctgctcgaggttatcaaacaagtttgtacaaaaaagcaggctccgcggccgcccccttcaccatggatgctgattctgcctccagttctgaatcagagagaaacatcactatcatggcttcagggaacacaggtggtgaaaaagatggccttcggaatagcactggacttggttcccaaaacaagtttgtagttggtagcagcagcaataatgtgggccatggaagtagtactgggccatggggtttttcccatggagccataataagcacatgtcaggtctctgtggatgctcctgaaagcaaatctgaaagtagcaacaatagaatgaatgcttggggcactgtaagttcttcatcaaatggagggttaaatccaagcactttgaattcagctagcaaccatggtgcctggccagtattagagaacaatggacttgccctaaaagggcctgtagggagtggtagttctggcattaatattcagtgcagtactataggccagatgcctaacaatcagagtattaactctaaagtgagtggtggttctacccatggtacctggggaagccttcaggaaacttgtgaatctgaagtaagtggtacacagaaggtttcattcagtggtcaacctcaaaatattaccactgaaatgactggaccaaataacactactaactttatgacctctagtttaccaaactccggttcagtgcagaataatgagctgcctagtagtaacacaggggcctggcgtgtgagcacaatgaatcatcctcagatgcaggctccatcaggtatgaatggcacttccctttctcaccttagcaatggagagtcaaaaagtggaggctcttatggtactacatggggtgcctatggttctaattactctggagacaaatgttcaggccctaatggccaagctaatggtgacactgtgaatgcaactctaatgcagcctggcgtaaatggtcctatgggcactaactttcaagttaacacaaacaaaggaggtggtgtgtgggaatctggtgcagcaaactcccagagtacatcatggggaagtggaaatggcgcaaattctggaggaagtcgaagaggatggggaacccctgcacaaaacactggcactaatttacccagcgttgagtggaacaaactgcctagcaatcagcattccaatgatagtgcaaatggcaatggtaagacgtttacaaatggatggaaatctactgaggaagaggatcagggttctgccacatctcagacaaatgagcaaagcagtgtgtgggccaaaacaggaggtacagtggagagcgatggtagtacagaaagcactggacgccttgaggaaaaaggaactggggaaagtcagagtagagacagaagaaaaattgatcagcacacattactccaaagcattgtaaacagaactgacttagatccacgtgtcctgtccaactctggttggggacagactcctattaagcagaatactgcctgggatacagaaacatcacctagaggggaacgaaagactgacaatgggacagaggcctggggaagctctgcaacacagacttttaactcaggggcatgtatagataagactagccctaatggtaatgatacctcatctgtatcagggtggggcgatcccaaacctgctctgaggtggggagattccaaaggctcaaactgccagggggggtgggaagatgattctgctgctacaggaatggtcaagagcaatcagtgggggaattgcaaagaggagaaggctgcatggaatgactcgcaaaagaataaacagggatggggtgatggacaaaaatcaagccaagggtggtctgtttctgccagtgataactggggagaaacttcaaggaataaccattggggtgaggccaataagaaatccagctcaggaggtagtgacagtgacaggtccgtttccggttggaacgaacttggtaaaactagttctttcacttggggaaacaacataaatccaaataattcatcaggatgggatgaatcttctaaacctactccttcccagggatggggagaccctccaaagtctaatcagtctctaggttggggagattcgtcaaagccagtcagctctccagactggaacaagcaacaagacattgttggatcttggggaatcccaccagctacaggcaaacctcctggtacaggctggctggggggacctataccagccccagcaaaagaagaagaacccacaggctgggaggaaccatccccagaatctatacgtcgcaaaatggagattgatgatggaacttcagcttggggagatccaagcaaatacaactacaaaaatgtgaacatgtggaacaaaaacgtcccaaatggcaacagccgttcagaccagcaagcacaggtacatcagctgctaacgcctgcaagtgccatctcaaacaaagaggcaagcagtggctctggctggggtgagccctggggggagccttctactccagccacaactgtggataatggtacttcagcatggggtaagcccatagacagtggtcccagctggggggaacccattgctgcggcatccagcacatccacgtggggctccagctctgttggtccacaagcattaagcaaatctgggccaaaatctatgcaagatggctggtgtggtgatgatatgccattgcctggaaatcgccccactggctgggaagaggaagaggatgtggagattggaatgtggaatagtaattcatctcaagagcttaactcatctttaaattggccaccatatacaaagaaaatgtcatcgaagggtctgagtggcaaaaaaaggagaagggaaaggggaatgatgaaaggtggaaacaaacaagaagaagcgtggataaatccatttgttaaacagttttcaaacatcagtttttcgagagactcaccagaggaaaatgtacaaagcaataagatggacctttctggaggaatgttacaagacaaacgaatggagatagataaacatagcctaaatattggtgattacaatcgaacggtcgggaaaggccctggttctcggcctcagatttccaaagagtcttccatggagcgcaatccttattttgataaggatggcattgtagcagatgaatcccaaaacatgcagtttatgtccagtcaaagcatgaagcttcccccttcaaatagtgcactacctaaccaggcccttggctccatagcagggctgggtatgcaaaacttgaattctgttagacagaatggcaatcccagtatgtttggtgttggaaacacagcagcacaaccccggggcatgcagcagcctccagcacaacctcttagttcatctcagcctaatctccgtgctcaagtgcctcctccattactctcccctcaggttccagtttcattgctgaagtatgcaccaaacaacggtggcctgaatccactctttggccctcaacaggtagccatgctgaaccagctatcccagctaaaccagctttctcagatctcccagttacagcgattgttagcgcagcagcaaagggcgcagagtcagagaagcgtgccttctgggaaccggccgcagcaagaccagcagggtcgacctcttagtgtgcagcagcaaatgatgcaacaatctcgtcaacttgatccaaacctgttggtgtagaagggtgggcgcgccgacccagctttcttgtacaaagtggttcgataacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgcatcgatggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 27 - DNA, 11498 bp, Artificial Sequence
  Lentiviral vector
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggcggccgcagatctctcgaggttgatctgccaccatggactacaaggatgacgatgacaagctcgatggaggatacccatacgatgttccagattacgctgctcgaggttatcaaacaagtttgtacaaaaaagcaggctccgcggccgcccccttcaccatgagagagaaggagcaagaaagggaagaacagttaatggaagacaagaaaaggaagaaagaggataaaaagaaaaaagaagccactcagaaggtcacggaacaaaaaaccaaagtgcccgaagtgacgaaaccaagtttaagccaaccaacggccgccagcccaattggcagctctccatcgccaccagtcaatggtggcaacaatgccaaaagggtggcagtgccgaacggacaaccgccaagcgccgcccgctacatgcctcgggaggtgccgccgcgattccgttgccagcaggaccacaaagtgttactaaaacgtgggcagccccctccaccgtcctgcatgctccttgggggtggggcagggcctcctccctgcacagcacctggagcaaacccaaacaacgcacaagtgacaggagcgctgctgcagagtgagagtgggactgcgccagactcaacccttggaggtgctgctgcttcaaattatgcaaattccacttggggctcgggagcctcctccaacaacggcacctcccccaacccaattcacatctgggacaaggtgattgtagacgggtctgacatggaagagtggccttgtattgccagcaaagacactgaatcttcttccgaaaacaccaccgataacaacagtgcctcgaaccctggctctgagaagagcactctgccaggaagcaccactagtaacaaaggaaaagggagccagtgccagtctgcaagttctgggaacgaatgtaatcttggggtctggaaatctgaccctaaggctaaatctgttcaatcttccaactctactacagagaacaacaatggactaggaaattggaggaatgtgagtggtcaggatagaattggacctggctctggcttcagcaactttaacccaaatagcaacccatctgcctggccagcactggtccaagaaggaacttctaggaaaggggcattggaaacagataatagtaattccagtgcacaggttagcacagtaggtcagacatccagggaacagcagtcaaagatggaaaatgcgggtgttaattttgttgtctctggcagagaacaggctcaaattcataacactgatggaccaaaaaatggaaacactaactccttgaacttaagttcaccaaaccccatggagaataagggaatgccctttggaatgggcttggggaacacctccaggagcactgatgccccttcacaaagcactggagatcgaaagactgggagtgttggatcttggggtgcagctagggggccttctggaactgacacagtctctggacaaagcaattctggaaacaatgggaacaatggaaaagagagagaggactcctggaaaggagcttctgttcagaaatcaactgggtcaaaaaatgactcttgggacaacaataacaggtctacgggtgggtcctggaactttggcccccaggactctaatgacaacaaatggggtgaagggaacaaaatgacatctggggtctctcagggagaatggaaacagccgactgggtctgatgagttgaaaattggagaatggagtggtccaaaccaaccaaattctagcactggagcatgggacaatcaaaagggccaccccctccctgaaaaccaaggcaatgcccaggctccctgttggggaagatcttccagctccacaggaagtgaagttggaggtcaaagcactggaagcaaccacaaagcaggaagtagtgacagtcataactctggccgtcggtcgtacaggcccacacatcctgattgtcaggctgtcttgcagactcttttgagccgaactgatttggaccccagggtgctctcaaacactggctggggccaaactcaaattaagcaggacacagtgtgggacattgaagaggtgccaaggcctgaggggaaatctgacaaaggaactgaggggtgggagagcgctgccacacagaccaagaactcagggggctggggagatgcacccagccaaagcaatcaaatgaagtctggatggggggagctctcagcctctacagagtggaaagaccccaagaacacaggaggctggaatgactacaagaacaacaactcttccaactggggaggaggacgacctgatgaaaagacaccttcctcttggaatgagaatcccagcaaggatcaggggtggggaggtggacgccagcccaatcaaggatggtcttctggaaagaatggttggggggaggaagtcgatcagacaaaaaacagcaattgggaaagttctgcaagtaaacctgtgtctgggtggggtgaaggagggcagaatgaaatcgggacttggggtaatggtggcaatgcaagcctagcttcaaaaggtgggtgggaggattgcaaaagatccccagcatggaatgagacgggccgacagcccaattcctggaataaacaacaccaacagcagcagcccccacagcagccgccgccaccacaaccagaggcttctggttcgtggggaggcccacccccaccacctccaggcaacgttcgaccttccaattccagctggagcagcgggccacagcctgcaacacctaaggatgaggaacccagtggttgggaagagccatccccacagtcaattagtcggaaaatggacattgatgatggcacttcagcatggggagaccctaacagttataactacaagaatgtgaatctgtgggataagaattcccaagggggcccagcacctcgagaaccaaacctgcccaccccaatgaccagtaaatcggcatcagattccaaatctatgcaagacggctggggggagagtgacgggccagtcacaggagctcgccatcccagctgggaagaggaggaggatggaggagtctggaacaccactggctctcagggcagtgcttcctcccacaactcagcaagctggggacaaggaggaaagaaacaaatgaagtgctcactcaaaggaggaaacaatgattcatggatgaatcctcttgccaaacagttttcaaatatgggattgctgagtcagactgaagataatccaagcagcaaaatggatttgtctgtaggaagcctttcagataaaaaatttgatgtggacaagcgagcgatgaatctcggggattttaatgatatcatgaggaaggatcgatctgggttccgtccacctaattccaaagacatgggaaccacagatagtgggccttattttgagaagggcggtagtcatggtttgtttggaaacagcacagcacaatcgagaggtctgcacacacccgtgcagccactaaattcttctcccagtctccgggcgcaagtgcctccccagtttatttccccccaggtttctgcctcaatgctcaagcagtttcccaacagtggcctgagtccaggtcttttcaatgtggggccccagttatctcctcaacaaattgccatgctgagccagcttccacaaattccccagtttcagttggcatgtcagcttctcttgcagcagcagcaacagcagcagttgttacagaaccagagaaagatttctcaagctgtacgccaacagcaagagcagcagctggctcgaatggtgagtgcactgcagcagcagcagcagcagcagcagaggcagccaggcatgtagaagggtgggcgcgccgacccagctttcttgtacaaagtggttcgataacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgcatcgatggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 28 - DNA, 11471 bp, Artificial Sequence
  Lentiviral vector
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggcggccgcagatctctcgaggttgatctgccaccatggactacaaggatgacgatgacaagctcgatggaggatacccatacgatgttccagattacgctgctcgaggttatcaaacaagtttgtacaaaaaagcaggctccgcggccgcccccttcaccatggctacagggagtgcccagggcaacttcactggacataccaagaagacaaatggcaataatggcaccaatggcgcactcgtccaaagcccttctaatcagagtgcccttggagcagggggagcgaacagtaatggaagtgcggccagagtgtggggtgtagccacaggctccagctctggcctggctcactgctctgtcagtggtggggatggaaaaatggacactatgattggagatgggagaagtcagaattgctggggtgcttccaactccaatgctggcattaatcttaaccttaatcctaatgccaacccagctgcctggcctgtacttggacatgaaggaaccgtggcgacaggcaacccttccagtatttgcagtccagtcagtgccataggtcaaaatatgggcaaccagaacgggaacccaacaggcactttaggtgcttggggaaacttgctgccacaagagagcacagaaccacaaacgtccacttctcagaatgtgtctttcagcgcacaacctcagaaccttaacactgatggaccaaataacactaaccccatgaactcttcacccaaccctatcaatgcaatgcagacaaatggactgccaaactggggcatggctgttggtatgggggccatcatcccgccccacctgcaaggccttcctggtgctaatggatcatcagtttctcaagtcagtgggggcagtgctgaaggaataagcaattctgtgtggggactgtccccaggtaaccctgccacaggaaatagcaattctgggttcagtcaggggaatggagacactgtgaactcagcattaagtgctaaacaaaatggatccagcagtgctgtgcaaaaggaaggaagtggaggaaatgcttgggattcaggacctcctgctggtcctggaatactcgcctggggaaggggcagtggcaacaatggcgttggtaatatccattcaggagcttggggccaccccagccgaagcacctctaacggtgtgaatggggaatggggaaagcccccaaaccagcattccaacagtgacatcaatgggaaaggatcaacagggtgggagagtcctagtgtcaccagccagaaccctaccgtacagcctggtggtgaacacatgaactcctgggccaaagcggcatcttctggaactacagcaagtgaaggaagtagtgatggttctggcaaccacaatgaaggaagcactgggagggaaggaacgggagaaggccgaaggcgagataaagggattatagaccaagggcacatccagttgccaaggaatgatcttgacccaagagttctgtctaatactggttggggacagactcctgtaaagcaaaacactgcctgggaatttgaagaatcccctaggtctgaaaggaaaaatgacaatgggacagaggcctggggttgtgcagctactcaggcttcaaactcaggggggaagaacgatgggtccatcatgaacagtacaaatacctcttcagtatctgggtgggtcaacgcgccacctgccgctgtgccagcaaacacaggttggggagacagcaacaacaaagcgccaagtggcccgggggtttggggggactcgataagctctactgctgttagtactgctgctgctgccaagagtggccatgcttggagtggggccgcaaatcaggaggacaagtcacccacctggggtgagcctccaaagcccaaatcccaacactggggagatggacaaagatcaaatccagcctggagtgcaggagggggagattgggcagattcatcgtctgtccttggacacttgggggatgggaaaaaaaatggatctggatgggatgctgacagtaataggtcagggtctggttggaatgacaccacgagatctgggaacagtggctggggcaacagcacaaatacaaaggccaatccaggtacaaactggggggagactttaaaacctggcccccaacagaactgggctagcaaaccccaagacaacaatgtgagtaactggggaggagctgcttctgtgaaacagacaggaacagggtggatcggggggccggtaccggtcaaacagaaggacagcagtgaagcaactggctgggaagaaccctctccaccgtccattcgccgcaaaatggaaattgatgatggtacctcagcttggggggacccaagcaactataacaataaaactgtaaacatgtgggatagaaacaacccggtcatccagagcagtaccacgaccaataccaccaccaccaccaccactaccacgagcaacaccacacacagggtcgagacgccgcccccgcaccaggctggtactcagctgaatcgatcaccgttgcttggtccaggtaggaaagtttcatcaggctggggagaaatgcctaatgttcactcaaagactgaaaactcttggggagaaccatcctccccttctaccctggtggataatggcacagcagcatgggggaagccacccagcagtggcagcgggtggggagatcaccctgccgagccgccggtggcatttggaagagctggcgcacctgttgctgcctcagccctgtgcaaaccagcttcaaaatctatgcaagaaggctggggcagtggtggggatgaaatgaacctcagtaccagccagtgggaggatgaagaaggggacgtgtggaataatgctgcttcccaagaaagcacctcctcctgcagctcctgggggaacgcccccaaaaaaggacttcaaaagggcatgaagacgtctggcaagcaggatgaggcctggatcatgagccggctgatcaaacaactcacagacatgggcttcccgagagagccagctgaggaggccttgaagagtaacaatatgaatcttgatcaggccatgagcgctctgctggaaaagaaggtggacgtggacaagcgtgggctgggagtgaccgaccataatggaatggccgccaagcccctcggctgccgcccgccaatctccaaagagtcttccgtggaccgccccacctttcttgacaaggatggcggcctcgtggaagagcccacgccttcaccgttcttgccttccccaagcctgaagctccccctttcacacagtgcactccccagtcaggccctgggtgggattgcctccgggctgggcatgcaaaacttgaattcttctagacagataccgagtggcaatctgggtatgtttggcaatagtggagcagcacaagccaggaccatgcagcagccgccacagccaccagtgcagcctcttaactcttcccagcccagtctccgtgctcaagtgcctcagtttctatcccctcaggttcaagcacagcttttgcagtttgcagcaaaaaacattggtctcaaccctgcactattaacctcgccaattaatcctcaacatatgacgatgttgaaccagctctatcagctgcagctggcataccaacgtttacaaatccagcagcagatgttacaggcccagcgtaatgtgtccggatccatgagacaacaggagcagcaagttgcgcgcacaatcactaatctgcagcagcagatccagcagcaccagcgccagctggcccaggccctgctcgtgaagcagtagaagggtgggcgcgccgacccagctttcttgtacaaagtggttcgataacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgcatcgatggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 29 - protein, 857 aa, Homo sapiens
MEAGPSGAAAGAYLPPLQQVFQAPRRPGIGTVGKPIKLLANYFEVDIPKIDVYHYEVDIKPDKCPRRVNREVVEYMVQHFKPQIFGDRKPVYDGKKNIYTVTALPIGNERVDFEVTIPGEGKDRIFKVSIKWLAIVSWRMLHEALVSGQIPVPLESVQALDVAMRHLASMRYTPVGRSFFSPPEGYYHPLGGGREVWFGFHQSVRPAMWKMMLNIDVSATAFYKAQPVIEFMCEVLDIRNIDEQPKPLTDSQRVRFTKEIKGLKVEVTHCGQMKRKYRVCNVTRRPASHQTFPLQLESGQTVECTVAQYFKQKYNLQLKYPHLPCLQVGQEQKHTYLPLEVCNIVAGQRCIKKLTDNQTSTMIKATARSAPDRQEEISRLMKNASYNLDPYIQEFGIKVKDDMTEVTGRVLPAPILQYGGRNRAIATPNQGVWDMRGKQFYNGIEIKVWAIACFAPQKQCREEVLKNFTDQLRKISKDAGMPIQGQPCFCKYAQGADSVEPMFRHLKNTYSGLQLIIVILPGKTPVYAEVKRVGDTLLGMATQCVQVKNVVKTSPQTLSNLCLKINVKLGGINNILVPHQRSAVFQQPVIFLGADVTHPPAGDGKKPSITAVVGSMDAHPSRYCATVRVQRPRQEIIEDLSYMVRELLIQFYKSTRFKPTRIIFYRDGVPEGQLPQILHYELLAIRDACIKLEKDYQPGITYIVVQKRHHTRLFCADKNERIGKSGNIPAGTTVDTNITHPFEFDFYLCSHAGIQGTSRPSHYYVLWDDNRFTADELQILTYQLCHTYVRCTRSVSIPAPAYYARLVAFRARYHLVDKEHDSGEGSHISGQSNGRDPQALAKAVQVHQDTLRTMYFA

SEQ ID NO: 30 - protein, 859 aa, Homo sapiens
MYSGAGPALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 31 - RNA, 22 nt, Homo sapiens
uaacagucuacagccauggucg

SEQ ID NO: 32 - RNA, 20 nt, Homo sapiens
uaaggcacgcggugaaugcc

SEQ ID NO: 33 - RNA, 23 nt, Homo sapiens
aacauucauuguugucggugggu

SEQ ID NO: 34 - RNA, 24 nt, Artificial Sequence
  Negative control miRNA
augugguccaaccgacuaauacag

SEQ ID NO: 35 - DNA, 63 bp, Artificial Sequence
  oligo-dT-T7 primer
ggccagtgaattgtaatacgactcactatagggaggcggtttttttttttttttttttttttt

SEQ ID NO: 36 - DNA, 23 bp, Artificial Sequence
  Primer hZDHHC9
ccgtcgtgggactgactggattt

SEQ ID NO: 37 - DNA, 23 bp, Artificial Sequence
  Primer hZDHHC9
ttctggacgcgattcttccctgt

SEQ ID NO: 38 - DNA, 24 bp, Artificial Sequence
  Primer hZDHHC17
tggctgctcagttcggacatacct

SEQ ID NO: 39 - DNA, 23 bp, Artificial Sequence
  Primer for hZDHHC17
gctgcccacattaaaggcgtcat

SEQ ID NO: 40 - DNA, 27 bp, Artificial Sequence
  Primer for hGAPDH
catgacaactttggtatcgtggaagga

SEQ ID NO: 41 - DNA, 23 bp, Artificial Sequence
  Primer for hGAPDH
cacagtcttctgggtggcagtga

SEQ ID NO: 42 - DNA, 23 bp, Artificial Sequence
  Primer for hHBEGF
tgaaagcccaaggtgctgatgtc

SEQ ID NO: 43 - DNA, 23 bp, Artificial Sequence
  Primer for hHBEGF
agctacaggcatggaagcccaac

SEQ ID NO: 44 - DNA, 23 bp, Artificial Sequence
  Primer for hSTMN1
gcgcccaacaaagacagaatcaa

SEQ ID NO: 45 - DNA, 23 bp, Artificial Sequence
  Primer for hSTMN1
tggtgccatgaaggaggaagaga

SEQ ID NO: 46 - DNA, 23 bp, Artificial Sequence
  Primer for hTJAP1
tgcagaacagctacacggcttcc

SEQ ID NO: 47 - DNA, 23 bp, Artificial Sequence
  Primer for hTJAP1
cagctccacaatctcccagtcca

SEQ ID NO: 48 - DNA, 23 bp, Artificial Sequence
  Primer for hCDKN1A
gtgtcctggttcccgtttctcca

SEQ ID NO: 49 - DNA, 23 bp, Artificial Sequence
  Primer for hCDKN1A
ttcagcattgtgggaggagctgt

SEQ ID NO: 50 - DNA, 23 bp, Artificial Sequence
  Primer for AcGFP
catgaccgacaaggccaagaatg

SEQ ID NO: 51 - DNA, 23 bp, Artificial Sequence
  Primer for AcGFP
atcctcgatgttgtggcggatct

SEQ ID NO: 52 - DNA, 22 bp, Artificial Sequence
  Primer for DsRedEx1
cccagttccagtacggctccaa

SEQ ID NO: 53 - DNA, 22 bp, Artificial Sequence
  Primer for DsRedEx1
agttcatcacgcgctcccactt

SEQ ID NO: 54 - DNA, 23 bp, Artificial Sequence
  Primer for hp120RasGAP
taaacgccttcgtcaggtcagca

SEQ ID NO: 55 - DNA, 23 bp, Artificial Sequence
  Primer for hp120RasGAP
ttgcccttcccttgcatgagttt

SEQ ID NO: 56 - DNA, 20 bp, Artificial Sequence
  Primer for hVamp3
gcagccaagttgaagaggaa

SEQ ID NO: 57 - DNA, 20 bp, Artificial Sequence
  Primer for hVamp3
cagttttgagttccgctggt

SEQ ID NO: 58 - DNA, 19 bp, Artificial Sequence
  Primer for hPlod3
gcggtgatgaactttgtgg

SEQ ID NO: 59 - DNA, 20 bp, Artificial Sequence
  Primer for hPlod3
gggaggagatcacacagtcg

SEQ ID NO: 60 - DNA, 20 bp, Artificial Sequence
  Primer for hCtdsp1
cctgcctcctatgtcttcca

SEQ ID NO: 61 - DNA, 20 bp, Artificial Sequence
  Primer for hCtdsp1
gcctgagcactgagtacacg

SEQ ID NO: 62 - DNA, 23 bp, Artificial Sequence
  Primer for hPTBP1
acattccgttgccttacccgatg

SEQ ID NO: 63 - DNA, 23 bp, Artificial Sequence
  Primer for hPTBP1
ctacagcgtccacagcgaacaca

SEQ ID NO: 64 - DNA, 20 bp, Artificial Sequence
  Primer for hLIF
gtttccctccttcctttcca

SEQ ID NO: 65 - DNA, 21 bp, Artificial Sequence
  Primer for hLIF
cccacagggtacattcatcaa

SEQ ID NO: 66 - DNA, 20 bp, Artificial Sequence
  Primer for hC11orf67
cagacagagcaggcagtgaa

SEQ ID NO: 67 - DNA, 20 bp, Artificial Sequence
  Primer for hC11orf67
caggtggaatggaagacacc

SEQ ID NO: 68 - DNA, 20 bp, Artificial Sequence
  Primer for hC17orf28
cgacgtgaagctgtttgaga

SEQ ID NO: 69 - DNA, 20 bp, Artificial Sequence
  Primer for hC17orf28
aagctcagaatggtggatgg

SEQ ID NO: 70 - DNA, 20 bp, Artificial Sequence
  Primer for hFAM82A1
gagtctggcaagtcggagag

SEQ ID NO: 71 - DNA, 19 bp, Artificial Sequence
  Primer for hFAM82A1
acgagcaaatcgccacata

SEQ ID NO: 72 - DNA, 19 bp, Artificial Sequence
  Primer for hTSKU
cctcatctggctgggatct

SEQ ID NO: 73 - DNA, 20 bp, Artificial Sequence
  Primer for hTSKU
ggagggtaagaagggctgtc

SEQ ID NO: 74 - DNA, 21 bp, Artificial Sequence
  Primer for hLGALS3
ccaatgcaaacagaattgctt

SEQ ID NO: 75 - DNA, 20 bp, Artificial Sequence
  Primer for hLGALS3
gaagcgtgggttaaagtgga

SEQ ID NO: 76 - DNA, 23 bp, Artificial Sequence
  Primer for hFCHO2
agctgcctctttaaccaagaatt

SEQ ID NO: 77 - DNA, 21 bp, Artificial Sequence
  Primer for hFCHO2
ttggaagacagatgtgcattg

SEQ ID NO: 78 - DNA, 20 bp, Artificial Sequence
  Primer for hLITAF
atgcttgggaaccagaactg

SEQ ID NO: 79 - DNA, 20 bp, Artificial Sequence
  Primer for hLITAF
tgacacagcagccaacctag

SEQ ID NO: 80 - DNA, 23 bp, Artificial Sequence
  Primer for HBRI3
gtaaagcgaggtgggaccgatgt

SEQ ID NO: 81 - DNA, 18 bp, Artificial Sequence
  Primer for HBRI3
ggcgggagcagcagcact

SEQ ID NO: 82 - DNA, 20 bp, Artificial Sequence
  Pirmer for hBVES
ccctctttccaatcctggtt

SEQ ID NO: 83 - DNA, 20 bp, Artificial Sequence
  Primer for hBVES
atgagcagaaccgaagcagt

SEQ ID NO: 84 - DNA, 20 bp, Artificial Sequence
  Primer for hC11orf67
cagacagagcaggcagtgaa

SEQ ID NO: 85 - DNA, 20 bp, Artificial Sequence
  Primer for hC11orf67
caggtggaatggaagacacc

SEQ ID NO: 86 - DNA, 20 bp, Artificial Sequence
  Primer for hC17orf28
cgacgtgaagctgtttgaga

SEQ ID NO: 87 - DNA, 20 bp, Artificial Sequence
  Primer for hC17orf28
aagctcagaatggtggatgg

SEQ ID NO: 88 - DNA, 20 bp, Artificial Sequence
  Primer for hC19orf54
gctggtctcgaactcctgac

SEQ ID NO: 89 - DNA, 21 bp, Artificial Sequence
  Primer for hC19orf54
acgcatcctctttaccctgat

SEQ ID NO: 90 - DNA, 24 bp, Artificial Sequence
  Primer for hC1D
agcgtgaaatgacttaaatgttca

SEQ ID NO: 91 - DNA, 21 bp, Artificial Sequence
  Primer for hC1D
gaggcaacatgccaagaatta

SEQ ID NO: 92 - DNA, 19 bp, Artificial Sequence
  Primer for hC20orf29
cacagccttccctcatctg

SEQ ID NO: 93 - DNA, 23 bp, Artificial Sequence
  Primer for hC20orf29
ggctagactgctcacattcatct

SEQ ID NO: 94 - DNA, 23 bp, Artificial Sequence
  Primer for hC22orf39
cctgatgctgtgctgtagtgtat

SEQ ID NO: 95 - DNA, 23 bp, Artificial Sequence
  Primer for hC22orf39
cataggaggttgtctagggaggt

SEQ ID NO: 96 - DNA, 20 bp, Artificial Sequence
  Primer for hCARM1
ggcagacacagacacctcaa

SEQ ID NO: 97 - DNA, 20 bp, Artificial Sequence
  Primer for hCARM1
ggactggagcgtctacaagg

SEQ ID NO: 98 - DNA, 23 bp, Artificial Sequence
  Primer for HCASK
tacaaagaatccaaacctttcca

SEQ ID NO: 99 - DNA, 23 bp, Artificial Sequence
  Primer for HCASK
agtatttgcagggtacagcagag

SEQ ID NO: 100 - DNA, 25 bp, Artificial Sequence
  Primer for hCCDC126
ttccaaatctctcttctcttctctg

SEQ ID NO: 101 - DNA, 21 bp, Artificial Sequence
  Primer for hCCDC126
ccaaacttctacctggcaatg

SEQ ID NO: 102 - DNA, 23 bp, Artificial Sequence
  Primer for HCCNY
tgtcttctgagctttcttcctca

SEQ ID NO: 103 - DNA, 24 bp, Artificial Sequence
  Primer for HCCNY
tggttcagcactacagtcaatttc

SEQ ID NO: 104 - DNA, 21 bp, Artificial Sequence
  Primer for hCDC42SE1
tttggtgctctcttgccttta

SEQ ID NO: 105 - DNA, 20 bp, Artificial Sequence
  Primer for hCDC42SE1
ctcaaacccattcccatcac

SEQ ID NO: 106 - DNA, 23 bp, Artificial Sequence
  Primer for hCDKN1A
gtgtcctggttcccgtttctcca

SEQ ID NO: 107 - DNA, 23 bp, Artificial Sequence
  Primer for hCDKN1A
ttcagcattgtgggaggagctgt

SEQ ID NO: 108 - DNA, 21 bp, Artificial Sequence
  Primer for HCDKN1A
gttcccgtttctccacctaga

SEQ ID NO: 109 - DNA, 23 bp, Artificial Sequence
  Primer for HCDKN1A
acagaacagtacagggtgtggtc

SEQ ID NO: 110 - DNA, 21 bp, Artificial Sequence
  Primer for hCDKN3
tcaccagagcaagccatagac

SEQ ID NO: 111 - DNA, 20 bp, Artificial Sequence
  Primer for hCDKN3
gcagctaatttgtcccgaaa

SEQ ID NO: 112 - DNA, 23 bp, Artificial Sequence
  Primer for hCEBPA
cacagaggccagatacaagtgtt

SEQ ID NO: 113 - DNA, 21 bp, Artificial Sequence
  Primer for hCEBPA
agggaccggagttatgacaag

SEQ ID NO: 114 - DNA, 20 bp, Artificial Sequence
  Primer for hCNKSR3
gaccatgaggctttccaaga

SEQ ID NO: 115 - DNA, 22 bp, Artificial Sequence
  Primer for hCNKSR3
cctgttcagtgtctgtcttcca

SEQ ID NO: 116 - DNA, 23 bp, Artificial Sequence
  Primer for HCOA5
cagtccgaagatgatggtaaaga

SEQ ID NO: 117 - DNA, 23 bp, Artificial Sequence
  Primer for HCOA5
tttaatagcgtccacaggcatag

SEQ ID NO: 118 - DNA, 19 bp, Artificial Sequence
  Primer for hCOMMD5
tgtcgtgggaagagtcagc

SEQ ID NO: 119 - DNA, 19 bp, Artificial Sequence
  Primer for hCOMMD5
attcagatccggcttggac

SEQ ID NO: 120 - DNA, 18 bp, Artificial Sequence
  Primer for hCOPS7A
ctgggaatgctgctgctt

SEQ ID NO: 121 - DNA, 21 bp, Artificial Sequence
  Primer for hCOPS7A
gggccaagaaagaatcatctc

SEQ ID NO: 122 - DNA, 20 bp, Artificial Sequence
  Primer for hCOX7A2L
tgatttccctggaggttctg

SEQ ID NO: 123 - DNA, 20 bp, Artificial Sequence
  Primer for hCOX7A2L
ccatagccacatccaaatcc

SEQ ID NO: 124 - DNA, 20 bp, Artificial Sequence
  Primer for hCPEB1
tgtgtgccaaagaaccagtg

SEQ ID NO: 125 - DNA, 20 bp, Artificial Sequence
  Primer for hCPEB1
atgcaacaatgcaacgtcag

SEQ ID NO: 126 - DNA, 19 bp, Artificial Sequence
  Primer for hCTC1
ggcaccagaacaccaaaga

SEQ ID NO: 127 - DNA, 20 bp, Artificial Sequence
  Primer for hCTC1
cctggctcctctccctactt

SEQ ID NO: 128 - DNA, 20 bp, Artificial Sequence
  Primer for hCtdsp1
cctgcctcctatgtcttcca

SEQ ID NO: 129 - DNA, 20 bp, Artificial Sequence
  Primer for hCtdsp1
gcctgagcactgagtacacg

SEQ ID NO: 130 - DNA, 19 bp, Artificial Sequence
  Primer for hCTDSP2
gggatctgcttcccactgt

SEQ ID NO: 131 - DNA, 20 bp, Artificial Sequence
  Primer for hCTDSP2
cttaatggcgtcctgcattt

SEQ ID NO: 132 - DNA, 23 bp, Artificial Sequence
  Primer for hDiablo
ttctgatgtgttctctgatctgc

SEQ ID NO: 133 - DNA, 24 bp, Artificial Sequence
  Primer for hDiablo
cctgtgttaagtcctgttgatgtt

SEQ ID NO: 134 - DNA, 21 bp, Artificial Sequence
  Primer for hDIMT1
agcaaatcctaaccagcacag

SEQ ID NO: 135 - DNA, 20 bp, Artificial Sequence
  Primer for hDIMT1
ctgcgttgaatccatgtagc

SEQ ID NO: 136 - DNA, 20 bp, Artificial Sequence
  Primer for hDTX2
ggttctgggtcagcttcttt

SEQ ID NO: 137 - DNA, 18 bp, Artificial Sequence
  Primer for hDTX2
cctgctggctttggctat

SEQ ID NO: 138 - DNA, 20 bp, Artificial Sequence
  Primer for hEBPL
tgttgctcctgaacaacctg

SEQ ID NO: 139 - DNA, 26 bp, Artificial Sequence
  Primer for hEBPL
tccctatatcaccatttaattgaaca

SEQ ID NO: 140 - DNA, 20 bp, Artificial Sequence
  Primer for hEIF2S3
tgcattgagatgggatttga

SEQ ID NO: 141 - DNA, 21 bp, Artificial Sequence
  Primer for hEIF2S3
caaccttcaaagtcagcgaag

SEQ ID NO: 142 - DNA, 21 bp, Artificial Sequence
  Primer for hELF4
catttgcacaaccaaacacag

SEQ ID NO: 143 - DNA, 19 bp, Artificial Sequence
  Primer for hELF4
gcgctctcgctcttctctt

SEQ ID NO: 144 - DNA, 20 bp, Artificial Sequence
  Primer for hELOVL1
gagtaagcagcctccacagg

SEQ ID NO: 145 - DNA, 21 bp, Artificial Sequence
  Primer for hELOVL1
cctcacacagaagaggtcagc

SEQ ID NO: 146 - DNA, 19 bp, Artificial Sequence
  Primer for hENHO
agggccatctggactatgc

SEQ ID NO: 147 - DNA, 20 bp, Artificial Sequence
  Primer for hENHO
agcctggacaccctcctatt

SEQ ID NO: 148 - DNA, 21 bp, Artificial Sequence
  Primer for hERAL1
gatttctcttcctgccctcac

SEQ ID NO: 149 - DNA, 24 bp, Artificial Sequence
  Primer for hERAL1
aggagacacgagttcttatccaag

SEQ ID NO: 150 - DNA, 20 bp, Artificial Sequence
  Primer for hERO1L
acaggcagatggattgagga

SEQ ID NO: 151 - DNA, 19 bp, Artificial Sequence
  Primer for hERO1L ′
caccacttctcacgcttgg

SEQ ID NO: 152 - DNA, 22 bp, Artificial Sequence
  Primer for hESYT2
gcctgtgtgtagctgtgtgttt

SEQ ID NO: 153 - DNA, 22 bp, Artificial Sequence
  Primer for hESYT2
accgctgttctctatttgcagt

SEQ ID NO: 154 - DNA, 20 bp, Artificial Sequence
  Primer for hEZH1
gaactgaagctgggacagga

SEQ ID NO: 155 - DNA, 21 bp, Artificial Sequence
  Primer for hEZH1
cgctgtgaggaataggtgaaa

SEQ ID NO: 156 - DNA, 22 bp, Artificial Sequence
  Primer for hFAIM
actaacattccaagggtcagga

SEQ ID NO: 157 - DNA, 24 bp, Artificial Sequence
  Primer for hFAIM
tccatatcttatttgccatttgtc

SEQ ID NO: 158 - DNA, 20 bp, Artificial Sequence
  Primer for hFAM120A
cccgcaagagctaagtagga

SEQ ID NO: 159 - DNA, 20 bp, Artificial Sequence
  Primer for hFAM120A
tcaatcaaacccaacagcag

SEQ ID NO: 160 - DNA, 22 bp, Artificial Sequence
  Primer for hFAM155B
gactctcaccgaaacacagagg

SEQ ID NO: 161 - DNA, 23 bp, Artificial Sequence
  Primer for hFAM155B
ttcttcctgagatttgagctgac

SEQ ID NO: 162 - DNA, 20 bp, Artificial Sequence
  Primer for hFAM60A
gagttggctctcccacagtc

SEQ ID NO: 163 - DNA, 20 bp, Artificial Sequence
  Primer for hFAM60A
ggaggatgataatggctgga

SEQ ID NO: 164 - DNA, 20 bp, Artificial Sequence
  Primer for hFAM82A1
gagtctggcaagtcggagag

SEQ ID NO: 165 - DNA, 19 bp, Artificial Sequence
  Primer for hFAM82A1
acgagcaaatcgccacata

SEQ ID NO: 166 - DNA, 23 bp, Artificial Sequence
  Primer for hFBLIM1
acttctgactttagcctcgtgct

SEQ ID NO: 167 - DNA, 23 bp, Artificial Sequence
  Primer for hFBLIM1
agcctacacacaacatggaagag

SEQ ID NO: 168 - DNA, 19 bp, Artificial Sequence
  Primer for hFBXO33
gcatgtgggcacttcttca

SEQ ID NO: 169 - DNA, 21 bp, Artificial Sequence
  Primer for hFBXO33
tcgtgaaatccctagcaacaa

SEQ ID NO: 170 - DNA, 23 bp, Artificial Sequence
  Primer for hFCHO2
agctgcctctttaaccaagaatt

SEQ ID NO: 171 - DNA, 21 bp, Artificial Sequence
  Primer for hFCHO2
ttggaagacagatgtgcattg

SEQ ID NO: 172 - DNA, 21 bp, Artificial Sequence
  Primer for hFERMT2
tgtgcactaaacaagcacgac

SEQ ID NO: 173 - DNA, 20 bp, Artificial Sequence
  Primer for hFERMT2
caattcatggccctaaggaa

SEQ ID NO: 174 - DNA, 22 bp, Artificial Sequence
  Primer for hFIS1
ctgtggccttcagctaatttct

SEQ ID NO: 175 - DNA, 24 bp, Artificial Sequence
  Primer for hFIS1
tttatttacactcatcccaaagca

SEQ ID NO: 176 - DNA, 21 bp, Artificial Sequence
  Primer for hFLT3LG
gccttaaacaacgcagtgaga

SEQ ID NO: 177 - DNA, 20 bp, Artificial Sequence
  Primer for hFLT3LG
ggcctctagccaacttcctc

SEQ ID NO: 178 - DNA, 28 bp, Artificial Sequence
  Primer for hFOS
ttctctttctccttagtcttctcatagc

SEQ ID NO: 179 - DNA, 22 bp, Artificial Sequence
  Primer for hFOS
acacactattgccaggaacaca

SEQ ID NO: 180 - DNA, 19 bp, Artificial Sequence
  Primer for hFOXP1
aaagtgggtgcagagctga

SEQ ID NO: 181 - DNA, 20 bp, Artificial Sequence
  Primer for hFOXP1
cccaaacatggtatgcagaa

SEQ ID NO: 182 - DNA, 23 bp, Artificial Sequence
  Primer for hFRMD8
ggttggagtgtgtgtgtctgagt

SEQ ID NO: 183 - DNA, 23 bp, Artificial Sequence
  Primer for hFRMD8
aaacgtaagaagctgaagggaaa

SEQ ID NO: 184 - DNA, 21 bp, Artificial Sequence
  Primer for hFZD6
catgaccacccattgattgta

SEQ ID NO: 185 - DNA, 20 bp, Artificial Sequence
  Primer for hFZD6
ctagtgagctgccgaaatga

SEQ ID NO: 186 - DNA, 20 bp, Artificial Sequence
  Primer for hGAD1
gccatcagacatgagggagt

SEQ ID NO: 187 - DNA, 20 bp, Artificial Sequence
  Primer for hGAD1
tggaaaccatgtgtgcagtt

SEQ ID NO: 188 - DNA, 27 bp, Artificial Sequence
  Primer for hGAPDH
catgacaactttggtatcgtggaagga

SEQ ID NO: 189 - DNA, 23 bp, Artificial Sequence
  Primer for hGAPDH
cacagtcttctgggtggcagtga

SEQ ID NO: 190 - DNA, 20 bp, Artificial Sequence
  Primer for hGDE1
aattaggcatggtggtgcat

SEQ ID NO: 191 - DNA, 20 bp, Artificial Sequence
  Primer for hGDE1
ccttgaacttctgggctcaa

SEQ ID NO: 192 - DNA, 23 bp, Artificial Sequence
  Primer for hGGA2
cagtaggtgctaagtgggaattg

SEQ ID NO: 193 - DNA, 23 bp, Artificial Sequence
  Primer for hGGA2
ggtggatatttgttgatggaaga

SEQ ID NO: 194 - DNA, 21 bp, Artificial Sequence
  Primer for hGHITM
tgggtatttggaaacaagtgg

SEQ ID NO: 195 - DNA, 20 bp, Artificial Sequence
  Primer for hGHITM
tgagaagcaacagcaggaga

SEQ ID NO: 196 - DNA, 20 bp, Artificial Sequence
  Primer for hGNAI3
tttccatcttcatggccttt

SEQ ID NO: 197 - DNA, 18 bp, Artificial Sequence
  Primer for hGNAI3
acagggatttggcaccac

SEQ ID NO: 198 - DNA, 21 bp, Artificial Sequence
  Primer for hGOLPH3
ctattgggaagaggcttgtga

SEQ ID NO: 199 - DNA, 21 bp, Artificial Sequence
  Primer for hGOLPH3
tgcaacatctgctaggactga

SEQ ID NO: 200 - DNA, 23 bp, Artificial Sequence
  Primer for hHBEGF
tgaaagcccaaggtgctgatgtc

SEQ ID NO: 201 - DNA, 23 bp, Artificial Sequence
  Primer for hHBEGF
agctacaggcatggaagcccaac

SEQ ID NO: 202 - DNA, 20 bp, Artificial Sequence
  Primer for hHMGB3
gtgttgtgggtgagtgttgc

SEQ ID NO: 203 - DNA, 20 bp, Artificial Sequence
  Primer for hHMGB3
ctgcgtgtttcatagcctca

SEQ ID NO: 204 - DNA, 20 bp, Artificial Sequence
  Primer for hHTT
atggctcagacgaggacact

SEQ ID NO: 205 - DNA, 20 bp, Artificial Sequence
  Primer for hHTT
caggttgccttcagttgtca

SEQ ID NO: 206 - DNA, 21 bp, Artificial Sequence
  Primer for hKDM6B
agcaacagacacaaggaccag

SEQ ID NO: 207 - DNA, 20 bp, Artificial Sequence
  Primer for hKDM6B
gtgagggaacccgtatgtga

SEQ ID NO: 208 - DNA, 20 bp, Artificial Sequence
  Primer for hKITLG
agtgtccacttgccaccatt

SEQ ID NO: 209 - DNA, 20 bp, Artificial Sequence
  Primer for hKITLG
agggtatatctgcgcatcca

SEQ ID NO: 210 - DNA, 21 bp, Artificial Sequence
  Primer for hKLHL15
tcaaatgccagagttcacaaa

SEQ ID NO: 211 - DNA, 21 bp, Artificial Sequence
  Primer for hKLHL15
ggtcaaagacacggaagagaa

SEQ ID NO: 212 - DNA, 21 bp, Artificial Sequence
  Primer for hLGALS3
ccaatgcaaacagaattgctt

SEQ ID NO: 213 - DNA, 20 bp, Artificial Sequence
  Primer for hLGALS3
gaagcgtgggttaaagtgga

SEQ ID NO: 214 - DNA, 20 bp, Artificial Sequence
  Primer for hLIF
gtttccctccttcctttcca

SEQ ID NO: 215 - DNA, 21 bp, Artificial Sequence
  Primer for hLIF
cccacagggtacattcatcaa

SEQ ID NO: 216 - DNA, 20 bp, Artificial Sequence
  Primer for hLITAF
atgcttgggaaccagaactg

SEQ ID NO: 217 - DNA, 20 bp, Artificial Sequence
  Primer for hLITAF
tgacacagcagccaacctag

SEQ ID NO: 218 - DNA, 20 bp, Artificial Sequence
  Primer for hLSM14A
aacacagttccgaggcattc

SEQ ID NO: 219 - DNA, 20 bp, Artificial Sequence
  Primer for hLSM14A
cgggacatctccaacagtct

SEQ ID NO: 220 - DNA, 20 bp, Artificial Sequence
  Primer for hLSM14B
ccacttggaaacaagccagt

SEQ ID NO: 221 - DNA, 20 bp, Artificial Sequence
  Primer for hLSM14B
ctccctccacccactgttac

SEQ ID NO: 222 - DNA, 20 bp, Artificial Sequence
  Primer for hMAD2L1BP
cctggtgagagaggaagcaa

SEQ ID NO: 223 - DNA, 20 bp, Artificial Sequence
  Primer for hMAD2L1BP
atgccatcagcctatcagga

SEQ ID NO: 224 - DNA, 22 bp, Artificial Sequence
  Primer for hMAPK1IP1L
agtggagttgagtgccatcttt

SEQ ID NO: 225 - DNA, 27 bp, Artificial Sequence
  Primer for hMAPK1IP1L
cttgatatttaagcaacacagtcacac

SEQ ID NO: 226 - DNA, 23 bp, Artificial Sequence
  Primer for hMBTPS1
gacaatggtgaaatcaagacctc

SEQ ID NO: 227 - DNA, 22 bp, Artificial Sequence
  Primer for hMBTPS1
ttgaaggaaacatgcacaaatc

SEQ ID NO: 228 - DNA, 19 bp, Artificial Sequence
  Primer for hMETTL2B
atccattgagcccaggagt

SEQ ID NO: 229 - DNA, 20 bp, Artificial Sequence
  Primer for hMETTL2B
gaattgtccagcatcaacga

SEQ ID NO: 230 - DNA, 20 bp, Artificial Sequence
  Primer for hMFSD3
tgctcatcctctctgccttt

SEQ ID NO: 231 - DNA, 20 bp, Artificial Sequence
  Primer for hMFSD3
attgaccactccagccactc

SEQ ID NO: 232 - DNA, 22 bp, Artificial Sequence
  Primer for hMLL3
tgcatccctacttcttcagtca

SEQ ID NO: 233 - DNA, 20 bp, Artificial Sequence
  Primer for hMLL3
cagcctccatttgggtgtat

SEQ ID NO: 234 - DNA, 20 bp, Artificial Sequence
  Primer for hMMGT1
ctcagtcgggaagagtcctg

SEQ ID NO: 235 - DNA, 20 bp, Artificial Sequence
  Primer for hMMGT1
ccaagtcatttggcctgatt

SEQ ID NO: 236 - DNA, 20 bp, Artificial Sequence
  Primer for hMRI1
tccgaaagtgctgggattac

SEQ ID NO: 237 - DNA, 20 bp, Artificial Sequence
  Primer for hMRI1
agtggaaccagggtgttgag

SEQ ID NO: 238 - DNA, 23 bp, Artificial Sequence
  Primer for hMTMR1
taactgggaacctcctgattctt

SEQ ID NO: 239 - DNA, 23 bp, Artificial Sequence
  Primer for hMTMR1
gaacacgacttagcaacaaatcc

SEQ ID NO: 240 - DNA, 20 bp, Artificial Sequence
  Primer for hMYADM
aatcggacgaagaaccacag

SEQ ID NO: 241 - DNA, 20 bp, Artificial Sequence
  Primer for hMYADM
gccaaagcaggacacgttat

SEQ ID NO: 242 - DNA, 21 bp, Artificial Sequence
  Primer for hNAA30
attggactgctgtttgactgg

SEQ ID NO: 243 - DNA, 21 bp, Artificial Sequence
  Primer for hNAA30
ttgcacatttcaaatcccatt

SEQ ID NO: 244 - DNA, 20 bp, Artificial Sequence
  Primer for hNDUFA11
tgcgtgtactttggcatagc

SEQ ID NO: 245 - DNA, 20 bp, Artificial Sequence
  Primer for hNDUFA11
tatttctggacgcattctgc

SEQ ID NO: 246 - DNA, 21 bp, Artificial Sequence
  Primer for hNFE2L2
tcagcatgctacgtgatgaag

SEQ ID NO: 247 - DNA, 21 bp, Artificial Sequence
  Primer for hNFE2L2
acattgccatctcttgtttgc

SEQ ID NO: 248 - DNA, 22 bp, Artificial Sequence
  Primer for hNFIC
catgtttcctaatttgcacgaa

SEQ ID NO: 249 - DNA, 23 bp, Artificial Sequence
  Primer for hNFIC
atacttattctcggaaggcaagg

SEQ ID NO: 250 - DNA, 21 bp, Artificial Sequence
  Primer for hNID1
acatggtcctcgaatcttgtg

SEQ ID NO: 251 - DNA, 21 bp, Artificial Sequence
  Primer for hNID1
cctgactttgtcctcacttgc

SEQ ID NO: 252 - DNA, 21 bp, Artificial Sequence
  Primer for hNREP
atgcactgcacttcttcgttt

SEQ ID NO: 253 - DNA, 21 bp, Artificial Sequence
  Primer for hNREP
cataaatgccacagatgcaga

SEQ ID NO: 254 - DNA, 20 bp, Artificial Sequence
  Primer for hNVL
acccactttcagctggacac

SEQ ID NO: 255 - DNA, 20 bp, Artificial Sequence
  Primer for hNVL
cagcttggcctcattcattt

SEQ ID NO: 256 - DNA, 20 bp, Artificial Sequence
  Primer for hORC5
tctgattggtctgggtggat

SEQ ID NO: 257 - DNA, 20 bp, Artificial Sequence
  Primer for hORC5
gtgctcaaaccctgctgttt

SEQ ID NO: 258 - DNA, 23 bp, Artificial Sequence
  hp120RasGAP
taaacgccttcgtcaggtcagca

SEQ ID NO: 259 - DNA, 23 bp, Artificial Sequence
  Primer for hp120RasGAP
ttgcccttcccttgcatgagttt

SEQ ID NO: 260 - DNA, 23 bp, Artificial Sequence
  Primer for hPADI2
ctgtgttcttcttgccatcttca

SEQ ID NO: 261 - DNA, 23 bp, Artificial Sequence
  Primer for hPADI2
taagttcctgcttccaccttgat

SEQ ID NO: 262 - DNA, 20 bp, Artificial Sequence
  Primer for hPARP3
tttgacacatctgcccagtc

SEQ ID NO: 263 - DNA, 23 bp, Artificial Sequence
  Primer for hPARP3
acagaaagacaaacactgcatga

SEQ ID NO: 264 - DNA, 19 bp, Artificial Sequence
  Primer for hPHTF2
tttcccagtggttgccata

SEQ ID NO: 265 - DNA, 20 bp, Artificial Sequence
  Primer for hPHTF2
gacatgggtgaagaggcaat

SEQ ID NO: 266 - DNA, 20 bp, Artificial Sequence
  Primer for hPIM2
ttgggaaggaatggaagatg

SEQ ID NO: 267 - DNA, 19 bp, Artificial Sequence
  Primer for hPIM2
cccaggagaacaaacagca

SEQ ID NO: 268 - DNA, 22 bp, Artificial Sequence
  Primer for hPKD2
tggagaaccaagagaatcctgt

SEQ ID NO: 269 - DNA, 22 bp, Artificial Sequence
  Primer for hPKD2
caacctggaaaggtctatttgc

SEQ ID NO: 270 - DNA, 22 bp, Artificial Sequence
  Primer for HPLA2G6
gagtgacctttgagagctgagg

SEQ ID NO: 271 - DNA, 22 bp, Artificial Sequence
  Primer for HPLA2G6
tgggactaaagaaatgggtgtc

SEQ ID NO: 272 - DNA, 21 bp, Artificial Sequence
  Primer for hPLAA
gtgttccaagagccacaagaa

SEQ ID NO: 273 - DNA, 20 bp, Artificial Sequence
  Primer for hPLAA
gaagggaaagcccaatgttt

SEQ ID NO: 274 - DNA, 19 bp, Artificial Sequence
  Primer for hPlod3
gcggtgatgaactttgtgg

SEQ ID NO: 275 - DNA, 20 bp, Artificial Sequence
  Primer for hPlod3
gggaggagatcacacagtcg

SEQ ID NO: 276 - DNA, 20 bp, Artificial Sequence
  Primer for hPLXNB2
tctgtctgtccaccacgaga

SEQ ID NO: 277 - DNA, 20 bp, Artificial Sequence
  Primer for hPLXNB2
gaggtcaggaaggcatcgta

SEQ ID NO: 278 - DNA, 20 bp, Artificial Sequence
  Primer for hPNKD
tctcatcgctaacaccacca

SEQ ID NO: 279 - DNA, 20 bp, Artificial Sequence
  Primer for hPNKD
acagtctcatcgcctgatcc

SEQ ID NO: 280 - DNA, 20 bp, Artificial Sequence
  Primer for hPOM121C
ggcctagcaatcaatcaagc

SEQ ID NO: 281 - DNA, 20 bp, Artificial Sequence
  Primer for hPOM121C
cctgcggaactgaggtaaac

SEQ ID NO: 282 - DNA, 21 bp, Artificial Sequence
  Primer for hPPP1R3B
cgatcacgtctctcctgacat

SEQ ID NO: 283 - DNA, 20 bp, Artificial Sequence
  Primer for hPPP1R3B
tgtccttcccagttccacat

SEQ ID NO: 284 - DNA, 23 bp, Artificial Sequence
  Primer for HPRR16
accactacaaccgtgtgatgtat

SEQ ID NO: 285 - DNA, 23 bp, Artificial Sequence
  Primer for HPRR16
ttgcttgagtagaaagtgctcat

SEQ ID NO: 286 - DNA, 20 bp, Artificial Sequence
  Primer for hPSMA2
ttcacggattcatggaacaa

SEQ ID NO: 287 - DNA, 20 bp, Artificial Sequence
  Primer for hPSMA2
tcccatggagacctatttgg

SEQ ID NO: 288 - DNA, 20 bp, Artificial Sequence
  Primer for hPSMD4
agccattcgaaatgctatgg

SEQ ID NO: 289 - DNA, 20 bp, Artificial Sequence
  Primer for hPSMD4
gctaccctttccctccagtc

SEQ ID NO: 290 - DNA, 23 bp, Artificial Sequence
  Primer for hPTBP1
acattccgttgccttacccgatg

SEQ ID NO: 291 - DNA, 23 bp, Artificial Sequence
  Primer for hPTBP1
ctacagcgtccacagcgaacaca

SEQ ID NO: 292 - DNA, 20 bp, Artificial Sequence
  Primer for hPTCD3
ctgttctgcactgagccaag

SEQ ID NO: 293 - DNA, 20 bp, Artificial Sequence
  Primer for hPTCD3
agcagtctccaagtcccaaa

SEQ ID NO: 294 - DNA, 26 bp, Artificial Sequence
  Primer for HPTF1A
ttatcctgttgagttgatgaaataga

SEQ ID NO: 295 - DNA, 18 bp, Artificial Sequence
  Primer for HPTF1A
ggccagagttctcccaac

SEQ ID NO: 296 - DNA, 20 bp, Artificial Sequence
  Primer for hRASEF
atctaaccgggaccaattcc

SEQ ID NO: 297 - DNA, 20 bp, Artificial Sequence
  Primer for hRASEF
cttcacaggccaaggatgtt

SEQ ID NO: 298 - DNA, 20 bp, Artificial Sequence
  Primer for hRASSF8
cccatagtgtgttgcctgtg

SEQ ID NO: 299 - DNA, 20 bp, Artificial Sequence
  Primer for hRASSF8
tccctgtgcatcaagacaaa

SEQ ID NO: 300 - DNA, 23 bp, Artificial Sequence
  Primer for hRBAK
tacgctgtccttaaagcttagca

SEQ ID NO: 301 - DNA, 20 bp, Artificial Sequence
  Primer for hRBAK
ttgaagaggcagctcagacc

SEQ ID NO: 302 - DNA, 20 bp, Artificial Sequence
  Primer for hRBM34
cccagattgcagatggattt

SEQ ID NO: 303 - DNA, 21 bp, Artificial Sequence
  Primer for hRBM34
ttccacagtccagaaagtgct

SEQ ID NO: 304 - DNA, 20 bp, Artificial Sequence
  Primer for hREEP3
ttaaccatgttgcccaggat

SEQ ID NO: 305 - DNA, 21 bp, Artificial Sequence
  Primer for hREEP3
acgcctgtaatcccagaagtt

SEQ ID NO: 306 - DNA, 21 bp, Artificial Sequence
  Primer for hRFX1
tcatggccttatctgttccag

SEQ ID NO: 307 - DNA, 18 bp, Artificial Sequence
  Primer for hRFX1
tatagcacgcggagcaca

SEQ ID NO: 308 - DNA, 20 bp, Artificial Sequence
  Primer for hRG9MTD1
cagaacacgtggctcaaatg

SEQ ID NO: 309 - DNA, 22 bp, Artificial Sequence
  Primer for hRG9MTD1
ccctacagtcagttgggaaaga

SEQ ID NO: 310 - DNA, 22 bp, Artificial Sequence
  Primer for hRHOG
agtcagtcagcaaatgcgtaag

SEQ ID NO: 311 - DNA, 23 bp, Artificial Sequence
  Primer for hRHOG
agaatcctgagaaggtgaatgtg

SEQ ID NO: 312 - DNA, 23 bp, Artificial Sequence
  Primer for hRIPK2
tgacatccaaggagaagaatttg

SEQ ID NO: 313 - DNA, 22 bp, Artificial Sequence
  Primer for hRIPK2
gctgaagacccatttgtttgtt

SEQ ID NO: 314 - DNA, 20 bp, Artificial Sequence
  Primer for hRNF125
tgcagcaattctgactcctg

SEQ ID NO: 315 - DNA, 20 bp, Artificial Sequence
  Primer for hRNF125
ataattcaggcgaccagcac

SEQ ID NO: 316 - DNA, 20 bp, Artificial Sequence
  Primer for hRNF13
aatcccgctcacatcagaac

SEQ ID NO: 317 - DNA, 21 bp, Artificial Sequence
  Primer for hRNF13
tgttgtaatcccgttcaccat

SEQ ID NO: 318 - DNA, 22 bp, Artificial Sequence
  Primer for hRNF135
cattgctgggagaattaagcat

SEQ ID NO: 319 - DNA, 23 bp, Artificial Sequence
  Primer for hRNF135
aggcacataaggtaaggtccaag

SEQ ID NO: 320 - DNA, 20 bp, Artificial Sequence
  Primer for hRNF182
aagctctggacatgggtacg

SEQ ID NO: 321 - DNA, 21 bp, Artificial Sequence
  Primer for hRNF182
actgccacaatacacggagac

SEQ ID NO: 322 - DNA, 19 bp, Artificial Sequence
  Primer for hRNPEPL1
gaaagtgggaggtggtgct

SEQ ID NO: 323 - DNA, 23 bp, Artificial Sequence
  Primer for hRNPEPL1
tttgcagagtggaagtttaatgg

SEQ ID NO: 324 - DNA, 21 bp, Artificial Sequence
  Primer for hRRAS
agtggcagtagcccagaagag

SEQ ID NO: 325 - DNA, 22 bp, Artificial Sequence
  Primer for hRRAS
ctcccaggacacatcacatacc

SEQ ID NO: 326 - DNA, 18 bp, Artificial Sequence
  Primer for hRSRC2
cgtgtccgctgtcttgtg

SEQ ID NO: 327 - DNA, 20 bp, Artificial Sequence
  Primer for hRSRC2
accacccagcttatctgtgc

SEQ ID NO: 328 - DNA, 20 bp, Artificial Sequence
  Primer for hSAP30L
gcgatcctttgaggttgtgt

SEQ ID NO: 329 - DNA, 20 bp, Artificial Sequence
  Primer for hSAP30L
cttgagctcatctgcccttc

SEQ ID NO: 330 - DNA, 20 bp, Artificial Sequence
  Primer for hSCAMP2
gcacaaccaccaccacataa

SEQ ID NO: 331 - DNA, 19 bp, Artificial Sequence
  Primer for hSCAMP2
ctttctctcgcctgccttc

SEQ ID NO: 332 - DNA, 20 bp, Artificial Sequence
  Primer for hSDF2
agacttgcgtgggtcagttc

SEQ ID NO: 333 - DNA, 20 bp, Artificial Sequence
  Primer for hSDF2
atgatgccaagctcctgaag

SEQ ID NO: 334 - DNA, 20 bp, Artificial Sequence
  Primer for hSERP2
gttctcaagcggaaaggaca

SEQ ID NO: 335 - DNA, 19 bp, Artificial Sequence
  Primer for hSERP2
gacccgcgtggctactaat

SEQ ID NO: 336 - DNA, 22 bp, Artificial Sequence
  Primer for hSERP2
tttcccttggtttcactaatgc

SEQ ID NO: 337 - DNA, 23 bp, Artificial Sequence
  Primer for hSERP2
acccacacatggtataaggttga

SEQ ID NO: 338 - DNA, 20 bp, Artificial Sequence
  Primer for hSH3BP5
tttgacccagtggaggctta

SEQ ID NO: 339 - DNA, 20 bp, Artificial Sequence
  Primer for hSH3BP5
aagtgcagctcatggtagcc

SEQ ID NO: 340 - DNA, 20 bp, Artificial Sequence
  Primer for hSIPA1L2
accattcaccacagcaggat

SEQ ID NO: 341 - DNA, 20 bp, Artificial Sequence
  Primer for hSIPA1L2
cgccagggtttacattcatc

SEQ ID NO: 342 - DNA, 24 bp, Artificial Sequence
  Primer for hSIX4
catcacctaacagtgcagtaaaga

SEQ ID NO: 343 - DNA, 23 bp, Artificial Sequence
  Primer for hSIX4
ctgtgccttacacaaagagaaac

SEQ ID NO: 344 - DNA, 20 bp, Artificial Sequence
  Primer for hSLC17A5
gctcaacaaccacagccact

SEQ ID NO: 345 - DNA, 23 bp, Artificial Sequence
  Primer for hSLC17A5
aaaggccacatcacctgagacta

SEQ ID NO: 346 - DNA, 23 bp, Artificial Sequence
  Primer for hSMARCAD1
aggtactgcatggaaataggtca

SEQ ID NO: 347 - DNA, 22 bp, Artificial Sequence
  Primer for hSMARCAD1
ttgaacaatggctctctcttca

SEQ ID NO: 348 - DNA, 20 bp, Artificial Sequence
  Primer for hSMN2
gagctgtgagaagggtgttg

SEQ ID NO: 349 - DNA, 21 bp, Artificial Sequence
  Primer for hSMN2
ccacatacgcctcacatacat

SEQ ID NO: 350 - DNA, 23 bp, Artificial Sequence
  Primer for hSNAI2
tctcaatctagccatcagcaaat

SEQ ID NO: 351 - DNA, 22 bp, Artificial Sequence
  Primer for hSNAI2
acacacacacccacagagagag

SEQ ID NO: 352 - DNA, 19 bp, Artificial Sequence
  Primer for hSOS2
aatgccggtatttgctgct

SEQ ID NO: 353 - DNA, 21 bp, Artificial Sequence
  Primer for hSOS2
ttccctccatcattgggttat

SEQ ID NO: 354 - DNA, 20 bp, Artificial Sequence
  Primer for hSPPL3
aaggtgcccattgttcagag

SEQ ID NO: 355 - DNA, 20 bp, Artificial Sequence
  Primer for hSPPL3
agcctttgttgtcaggcatc

SEQ ID NO: 356 - DNA, 23 bp, Artificial Sequence
  Primer for hSTMN1
gcgcccaacaaagacagaatcaa

SEQ ID NO: 357 - DNA, 23 bp, Artificial Sequence
  Primer for hSTMN1
tggtgccatgaaggaggaagaga

SEQ ID NO: 358 - DNA, 21 bp, Artificial Sequence
  Primer for hSTX2
gaaggaaaggtgggacatcac

SEQ ID NO: 359 - DNA, 23 bp, Artificial Sequence
  Primer for hSTX2
tgatttgtggctatgttgaaggt

SEQ ID NO: 360 - DNA, 20 bp, Artificial Sequence
  Primer for hTCTA
cctatgggaatgtgggtctg

SEQ ID NO: 361 - DNA, 20 bp, Artificial Sequence
  Primer for hTCTA
ggtccagatgggaaatgatg

SEQ ID NO: 362 - DNA, 20 bp, Artificial Sequence
  Primer for hTCTEX1D2
agcagcatttggctgtttct

SEQ ID NO: 363 - DNA, 24 bp, Artificial Sequence
  Primer for hTCTEX1D2
tcagatttcttcatggtcatgtct

SEQ ID NO: 364 - DNA, 23 bp, Artificial Sequence
  Primer for hTJAP1
tgcagaacagctacacggcttcc

SEQ ID NO: 365 - DNA, 23 bp, Artificial Sequence
  Primer for hTJAP1
cagctccacaatctcccagtcca

SEQ ID NO: 366 - DNA, 21 bp, Artificial Sequence
  Primer for hTLN1
aacggtgaagagagctgatga

SEQ ID NO: 367 - DNA, 23 bp, Artificial Sequence
  Primer for hTLN1
tattaacgctgctgtacctcgat

SEQ ID NO: 368 - DNA, 20 bp, Artificial Sequence
  Primer for hTMEM134
ggtgcctggagtctatcacg

SEQ ID NO: 369 - DNA, 21 bp, Artificial Sequence
  Primer for hTMEM134
gggcaggtagaagaactggaa

SEQ ID NO: 370 - DNA, 25 bp, Artificial Sequence
  Primer for hTMEM87A
gcagtgttgtaaataagaagctcgt

SEQ ID NO: 371 - DNA, 21 bp, Artificial Sequence
  Primer for hTMEM87A
gcgagaggtgtcagaacaaag

SEQ ID NO: 372 - DNA, 22 bp, Artificial Sequence
  Primer for hTMEM99
ttgctcttcaacaactggactg

SEQ ID NO: 373 - DNA, 21 bp, Artificial Sequence
  Primer for hTMEM99
aatggcctctggtaagaatgc

SEQ ID NO: 374 - DNA, 19 bp, Artificial Sequence
  Primer for hTOM1L1
cttgtggtggtggagaacg

SEQ ID NO: 375 - DNA, 20 bp, Artificial Sequence
  Primer for hTOM1L1
tattggtggcttccttctgg

SEQ ID NO: 376 - DNA, 21 bp, Artificial Sequence
  Primer for hTOMM70A
gaaactgaagcatgtgccatt

SEQ ID NO: 377 - DNA, 21 bp, Artificial Sequence
  Primer for hTOMM70A
gatgcagctttcagtgcagtt

SEQ ID NO: 378 - DNA, 24 bp, Artificial Sequence
  Primer for hTP53RK
catacacattcttctacccaacca

SEQ ID NO: 379 - DNA, 21 bp, Artificial Sequence
  Primer for hTP53RK
agctactccacctcctccaaa

SEQ ID NO: 380 - DNA, 24 bp, Artificial Sequence
  Primer for hTRPS1
tgcatgttcatttctactcacaaa

SEQ ID NO: 381 - DNA, 21 bp, Artificial Sequence
  Primer for hTRPS1
agacatctttgctgcctgaag

SEQ ID NO: 382 - DNA, 20 bp, Artificial Sequence
  Primer for hTSC22D2
aaattgccacgattcctctg

SEQ ID NO: 383 - DNA, 23 bp, Artificial Sequence
  Primer for hTSC22D2
acagctttgcttctttggttaca

SEQ ID NO: 384 - DNA, 19 bp, Artificial Sequence
  Primer for hTSKU
cctcatctggctgggatct

SEQ ID NO: 385 - DNA, 20 bp, Artificial Sequence
  Primer for hTSKU
ggagggtaagaagggctgtc

SEQ ID NO: 386 - DNA, 20 bp, Artificial Sequence
  Primer for hVamp3
gcagccaagttgaagaggaa

SEQ ID NO: 387 - DNA, 20 bp, Artificial Sequence
  Primer for hVamp3
cagttttgagttccgctggt

SEQ ID NO: 388 - DNA, 23 bp, Artificial Sequence
  Primer for hWDR47
tcatgcagacatagcatttcaag

SEQ ID NO: 389 - DNA, 20 bp, Artificial Sequence
  Primer for hWDR47
agaagcagagcagcagcagt

SEQ ID NO: 390 - DNA, 23 bp, Artificial Sequence
  Primer for hWIPF1
ggacaattcctttgcatatctga

SEQ ID NO: 391 - DNA, 21 bp, Artificial Sequence
  Primer for hWIPF1
tggcaactttgagctatcacc

SEQ ID NO: 392 - DNA, 24 bp, Artificial Sequence
  Primer for HYAP1
tgtactgacctgaaggagacctaa

SEQ ID NO: 393 - DNA, 23 bp, Artificial Sequence
  Primer for HYAP1
atcttatcccaatgctacccaat

SEQ ID NO: 394 - DNA, 24 bp, Artificial Sequence
  Primer for hZDHHC17
tggctgctcagttcggacatacct

SEQ ID NO: 395 - DNA, 23 bp, Artificial Sequence
  Primer for hZDHHC17
gctgcccacattaaaggcgtcat

SEQ ID NO: 396 - DNA, 20 bp, Artificial Sequence
  Primer for hZDHHC7
tacctgagccaccgtcctag

SEQ ID NO: 397 - DNA, 20 bp, Artificial Sequence
  Primer for hZDHHC7
ggatcacccacactttgtcc

SEQ ID NO: 398 - DNA, 23 bp, Artificial Sequence
  Primer for hZDHHC9
ccgtcgtgggactgactggattt

SEQ ID NO: 399 - DNA, 23 bp, Artificial Sequence
  Primer for hZDHHC9
ttctggacgcgattcttccctgt

SEQ ID NO: 400 - DNA, 20 bp, Artificial Sequence
  Primer for hZFP37
tctgtgttggcttctgcatc

SEQ ID NO: 401 - DNA, 20 bp, Artificial Sequence
  Primer for hZFP37
aatatcgctccctaccagca

SEQ ID NO: 402 - DNA, 21 bp, Artificial Sequence
  Primer for hZNF10
gtattgctgaagccaaccaga

SEQ ID NO: 403 - DNA, 20 bp, Artificial Sequence
  Primer for hZNF10
gctgccatgttcctcgtagt

SEQ ID NO: 404 - DNA, 20 bp, Artificial Sequence
  Primer for hZNF101
ccaccaatgtcaggaatgtg

SEQ ID NO: 405 - DNA, 20 bp, Artificial Sequence
  Primer for hZNF101
gaagggacgtgggacatcta

SEQ ID NO: 406 - DNA, 22 bp, Artificial Sequence
  Primer for hZNF12
ctgctttaccactgagctgaaa

SEQ ID NO: 407 - DNA, 21 bp, Artificial Sequence
  Primer for hZNF12
aacaggagccatgtgtgattt

SEQ ID NO: 408 - DNA, 25 bp, Artificial Sequence
  Primer for hZNF124
tttatgaaggtatcgaatgtgagaa

SEQ ID NO: 409 - DNA, 20 bp, Artificial Sequence
  Primer for hZNF124
atttcaatctggtgggcatt

SEQ ID NO: 410 - DNA, 20 bp, Artificial Sequence
  Primer for hZNF14
gcgtggtcaacatggtgtaa

SEQ ID NO: 411 - DNA, 20 bp, Artificial Sequence
  Primer for hZNF14
ttctcctgccttagcctcct

SEQ ID NO: 412 - DNA, 20 bp, Artificial Sequence
  Primer for hZNF268
cagtcttggcagcaacctct

SEQ ID NO: 413 - DNA, 20 bp, Artificial Sequence
  Primer for hZNF268
caccgttagggaatgtttcg

SEQ ID NO: 414 - DNA, 20 bp, Artificial Sequence
  Primer for hZNF283
tgtgaatgtaagggttgtgc

SEQ ID NO: 415 - DNA, 23 bp, Artificial Sequence
  Primer for hZNF283
atagcaagataattgcccataaa

SEQ ID NO: 416 - DNA, 20 bp, Artificial Sequence
  Primer for hZNF3
ctggtgatattgccagcaga

SEQ ID NO: 417 - DNA, 20 bp, Artificial Sequence
  Primer for hZNF3
cctcacagccggttactagc

SEQ ID NO: 418 - DNA, 20 bp, Artificial Sequence
  Primer for hZNF330
gcaggagtgagtgtgtgtgc

SEQ ID NO: 419 - DNA, 20 bp, Artificial Sequence
  Primer for hZNF330
ttgcttggcccattaagtgt

SEQ ID NO: 420 - DNA, 20 bp, Artificial Sequence
  Primer for hZNF367
agctgactggagaaacaagg

SEQ ID NO: 421 - DNA, 27 bp, Artificial Sequence
  Primer for hZNF367
aactaaagaaggaagggtagtaagaat

SEQ ID NO: 422 - DNA, 23 bp, Artificial Sequence
  Primer for hZNF443or799 ′
acacattggagatagaccctgtg

SEQ ID NO: 423 - DNA, 20 bp, Artificial Sequence
  Primer for hZNF443or799
ccagcaggtatcaagggatg

SEQ ID NO: 424 - DNA, 23 bp, Artificial Sequence
  Primer for hZNF449
aacagtgccttagaatggatgtg

SEQ ID NO: 425 - DNA, 23 bp, Artificial Sequence
  Primer for hZNF449
aattagtgcaagtgaagcaggaa

SEQ ID NO: 426 - DNA, 21 bp, Artificial Sequence
  Primer for hZNF562
gagaaatgtggctttgttcca

SEQ ID NO: 427 - DNA, 20 bp, Artificial Sequence
  Primer for hZNF562
gaaatctgggcaccttgaaa

SEQ ID NO: 428 - DNA, 20 bp, Artificial Sequence
  Primer for hZNF564
catgaagatggaggccttgt

SEQ ID NO: 429 - DNA, 21 bp, Artificial Sequence
  Primer for hZNF564
ctggcatagtccatgtctggt

SEQ ID NO: 430 - DNA, 20 bp, Artificial Sequence
  Primer for hZNF607
taatcacccagagggagctg

SEQ ID NO: 431 - DNA, 19 bp, Artificial Sequence
  Primer for hZNF607
ccagaatgagcccaaaggt

SEQ ID NO: 432 - DNA, 20 bp, Artificial Sequence
  Primer for hZNF655
tatgtggcgaacacaacctg

SEQ ID NO: 433 - DNA, 21 bp, Artificial Sequence
  Primer for hZNF655
caaggaaggaggaaaccagaa

SEQ ID NO: 434 - DNA, 21 bp, Artificial Sequence
  Primer for hZNF658
aaccctcacagtcttcctggt

SEQ ID NO: 435 - DNA, 20 bp, Artificial Sequence
  Primer for hZNF658
gcttccttgaccttgtgctc

SEQ ID NO: 436 - DNA, 19 bp, Artificial Sequence
  Primer for hZNF684
gcaacacatccgtgcttgt

SEQ ID NO: 437 - DNA, 22 bp, Artificial Sequence
  Primer for hZNF684
tctgaggagatgggacttcttg

SEQ ID NO: 438 - DNA, 20 bp, Artificial Sequence
  Primer for hZNF709
atgggtgtctgcttctccac

SEQ ID NO: 439 - DNA, 20 bp, Artificial Sequence
  Primer for hZNF709
ggaaacaccgacaatctgct

SEQ ID NO: 440 - DNA, 19 bp, Artificial Sequence
  Primer for hZNF746
ggctgaatgaatgggcact

SEQ ID NO: 441 - DNA, 25 bp, Artificial Sequence
  Primer for hZNF746
gtgctgttcctaccacacaaatatc

SEQ ID NO: 442 - DNA, 20 bp, Artificial Sequence
  Primer for hZNF780A
ggcatacctcgctgaattgt

SEQ ID NO: 443 - DNA, 20 bp, Artificial Sequence
  Primer for hZNF780A
aatggacctgatcgtcttgc

SEQ ID NO: 444 - DNA, 19 bp, Artificial Sequence
  Primer for hZNF823
ccttcactcgttcccgttt

SEQ ID NO: 445 - DNA, 21 bp, Artificial Sequence
  Primer for hZNF823
atgcaaggaacggagagaact

SEQ ID NO: 446 - DNA, 27 bp, Artificial Sequence
  Primer for hZNF850
tttgtctaaggattgtaacattgatga

SEQ ID NO: 447 - DNA, 25 bp, Artificial Sequence
  Primer for hZNF850
gacaattcaatctcatgaagaaacc